SEQUENCE LISTING (149 sequences)

SEQ ID NO: 1 - DNA, 50 bp, Canis lupus
  source 1..50: mol_type = genomic DNA
cgcaatgttcatcaagctgagccgagaagacattaatagcctgatgaata

SEQ ID NO: 2 - DNA, 50 bp, Canis lupus
  source 1..50: mol_type = genomic DNA
cgagagcagctcgatgcacatctgccactgctgcaacacctcctcgtgct

SEQ ID NO: 3 - DNA, 50 bp, Canis lupus
  source 1..50: mol_type = genomic DNA
tggtttcaaaaccgccgaatgaaactcaagaagatgagccgagagaaccg

SEQ ID NO: 4 - DNA, 50 bp, Canis lupus
  source 1..50: mol_type = genomic DNA
aagcaaatcagattccaggctgctgccaggtgttgtctcgtcttccgccg

SEQ ID NO: 5 - DNA, 50 bp, Canis lupus
  source 1..50: mol_type = genomic DNA
cgacaggcaggtcaagatttggtttcaaaaccgccgaatgaaactcaaga

SEQ ID NO: 6 - DNA, 50 bp, Canis lupus
  source 1..50: mol_type = genomic DNA
cggcacagactccagggttcacgcatatggccagacagatgtgtctggct

SEQ ID NO: 7 - DNA, 50 bp, Canis lupus
  source 1..50: mol_type = genomic DNA
cggaggagctgaccaattggtcatggtgctgaaacctttgtggggaatcg

SEQ ID NO: 8 - DNA, 50 bp, Canis lupus
  source 1..50: mol_type = genomic DNA
tcccagcctgggcaaaactttgatgataaatcaagcggcagatccctccg

SEQ ID NO: 9 - DNA, 50 bp, Canis lupus
  source 1..50: mol_type = genomic DNA
cgaaattaggtggtcgttggaatcctgatcgcagtaaagtggtccttggt

SEQ ID NO: 10 - DNA, 50 bp, Canis lupus
  source 1..50: mol_type = genomic DNA
cgtagccgaagctggagtgctgctttgctttcagtctcaggctggccagg

SEQ ID NO: 11 - DNA, 50 bp, Canis lupus
  source 1..50: mol_type = genomic DNA
cgctgtccagctgcagctgcgcctgaacctgaaaggacaagggcgtcacg

SEQ ID NO: 12 - DNA, 50 bp, Canis lupus
  source 1..50: mol_type = genomic DNA
cggtgacccacaggtactgcgcatggaacctaatgaccagtcactcaatt

SEQ ID NO: 13 - DNA, 50 bp, Canis lupus
  source 1..50: mol_type = genomic DNA
aaccaggtgcaagctgggaaacagtcccacattccttacagccagcaacg

SEQ ID NO: 14 - DNA, 50 bp, Canis lupus
  source 1..50: mol_type = genomic DNA
cgtctgggatggggaaagaccaaccagttggggctttctcccagggctcc

SEQ ID NO: 15 - DNA, 50 bp, Canis lupus
  source 1..50: mol_type = genomic DNA
acagatgcaaatactccaaagaagtgtcgggcactgttcgggcttgaccg

SEQ ID NO: 16 - DNA, 50 bp, Canis lupus
  source 1..50: mol_type = genomic DNA
aagtccgagagggggcctttcacatgacatcataaaagcctgatttatcg

SEQ ID NO: 17 - DNA, 50 bp, Canis lupus
  source 1..50: mol_type = genomic DNA
cgagtttgggggcatgacagtacatctgacccctttggggtaaaatttga

SEQ ID NO: 18 - DNA, 50 bp, Canis lupus
  source 1..50: mol_type = genomic DNA
atggccagtcattttgttcacaactttgcagcaagcaggagcaaaagccg

SEQ ID NO: 19 - DNA, 50 bp, Canis lupus
  source 1..50: mol_type = genomic DNA
taaactcctatgtatgttcacatctatgatctgctaaccattgctactcg

SEQ ID NO: 20 - DNA, 50 bp, Canis lupus
  source 1..50: mol_type = genomic DNA
cggatataattatcgaggagtcaagatgttatgctaaaaactatgcattg

SEQ ID NO: 21 - DNA, 50 bp, Canis lupus
  source 1..50: mol_type = genomic DNA
gtcttcctttcctgtactgacaagctgaacagacgcaccttgttggttcg

SEQ ID NO: 22 - DNA, 50 bp, Canis lupus
  source 1..50: mol_type = genomic DNA
cgtgtctgtttgcagcacccctggggcgagctgtggcttcctgtaacatg

SEQ ID NO: 23 - DNA, 50 bp, Canis lupus
  source 1..50: mol_type = genomic DNA
cgaaggtgaacttcttgatctggatgaccaggtagtcagggaatgaggca

SEQ ID NO: 24 - DNA, 50 bp, Canis lupus
  source 1..50: mol_type = genomic DNA
cggatctgagcacttgagactaccatttaatcaaatgaatcagtaactaa

SEQ ID NO: 25 - DNA, 50 bp, Canis lupus
  source 1..50: mol_type = genomic DNA
cgccatcccaggtggttgagttcagccaggttgagcacaacacagatgcc

SEQ ID NO: 26 - DNA, 50 bp, Canis lupus
  source 1..50: mol_type = genomic DNA
gtggaaggcggcgtgaagcggcggctcgtgctggcatctacggggatacg

SEQ ID NO: 27 - DNA, 50 bp, Canis lupus
  source 1..50: mol_type = genomic DNA
cgcgtacacccagacatcttcgggctgctattggattgactttgaaggtt

SEQ ID NO: 28 - DNA, 50 bp, Canis lupus
  source 1..50: mol_type = genomic DNA
gggccagtgtcgggacagttgtcagcagggctccaacatgacgctcatcg

SEQ ID NO: 29 - DNA, 50 bp, Canis lupus
  source 1..50: mol_type = genomic DNA
ccatgtacttgagatttctcatgacatcatcattaccttggtctcccgcg

SEQ ID NO: 30 - DNA, 50 bp, Canis lupus
  source 1..50: mol_type = genomic DNA
tcagccagcagagagattgcagccttcttccctgacttcagtgaacagcg

SEQ ID NO: 31 - DNA, 50 bp, Canis lupus
  source 1..50: mol_type = genomic DNA
aaaataaaactcagggaccaacatctgcttcctttgcacttgctccgtcg

SEQ ID NO: 32 - DNA, 50 bp, Canis lupus
  source 1..50: mol_type = genomic DNA
cgcactccctgtgacccaggacgtcatgatgcctgtctgttttctcaatg

SEQ ID NO: 33 - DNA, 50 bp, Canis lupus
  source 1..50: mol_type = genomic DNA
cgaagtaaaaaatgttgatcttgcatcaccagaggaacatcagaagcacc

SEQ ID NO: 34 - DNA, 50 bp, Canis lupus
  source 1..50: mol_type = genomic DNA
cgcagggagagattaagatctcgttgaaaaggaataaaaataacatcatc

SEQ ID NO: 35 - DNA, 50 bp, Canis lupus
  source 1..50: mol_type = genomic DNA
cgacgtgtccatcctgaccatcgaggatggcatcttcgaggtgaagtcca

SEQ ID NO: 36 - DNA, 50 bp, Canis lupus
  source 1..50: mol_type = genomic DNA
cgagtggcacatccgctccaaccagcagctggtgcccagctactctgagg

SEQ ID NO: 37 - DNA, 50 bp, Canis lupus
  source 1..50: mol_type = genomic DNA
ggtcatccacctgctgcagatggggcaggtgtggaggtaagagcactgcg

SEQ ID NO: 38 - DNA, 50 bp, Canis lupus
  source 1..50: mol_type = genomic DNA
ttagcacagtttaactccaccctcatttaaacttcctttgattctttccg

SEQ ID NO: 39 - DNA, 50 bp, Canis lupus
  source 1..50: mol_type = genomic DNA
ctgtcttcccacagggctccattgtgtgcagttcctgtttctcaggggcg

SEQ ID NO: 40 - DNA, 50 bp, Canis lupus
  source 1..50: mol_type = genomic DNA
cgaaagccgtgtgaactcttggtgaaccaagattgaagtcataaatcacg

SEQ ID NO: 41 - DNA, 50 bp, Canis lupus
  source 1..50: mol_type = genomic DNA
cgctgcatcgctgctctagggaagaggttaactgacagaatcacaatcca

SEQ ID NO: 42 - DNA, 50 bp, Canis lupus
  source 1..50: mol_type = genomic DNA
ttcagaccttctcaatatgattcacatttgcacatcaacagcctcatgcg

SEQ ID NO: 43 - DNA, 50 bp, Canis lupus
  source 1..50: mol_type = genomic DNA
cgagtaatgaaataatcatgtccagaaatgtatcaaaggccagagggatt

SEQ ID NO: 44 - DNA, 50 bp, Canis lupus
  source 1..50: mol_type = genomic DNA
tgtggtttccccgtgtgtgaggtgggatccactccccgcatagtctctcg

SEQ ID NO: 45 - DNA, 50 bp, Canis lupus
  source 1..50: mol_type = genomic DNA
caatgcttgaaggagcccaaatcaggagtctaattgtaatcaggaaatcg

SEQ ID NO: 46 - DNA, 50 bp, Canis lupus
  source 1..50: mol_type = genomic DNA
acgagctgcatgcatgccaaatcaggtcattcacaaatatagaacaaacg

SEQ ID NO: 47 - DNA, 50 bp, Canis lupus
  source 1..50: mol_type = genomic DNA
tgttaatttttccatctgctttggctgcaggtaatttggagacactgacg

SEQ ID NO: 48 - DNA, 50 bp, Canis lupus
  source 1..50: mol_type = genomic DNA
aaaatgcatcttgtttctttcaacccttgactgttttgacattttcttcg

SEQ ID NO: 49 - DNA, 50 bp, Canis lupus
  source 1..50: mol_type = genomic DNA
ttcttgcctctgaggagctgcccaatgactggaggtctgggattaaagcg

SEQ ID NO: 50 - DNA, 50 bp, Canis lupus
  source 1..50: mol_type = genomic DNA
ggagtgctgctttgctttcagtctcaggctggccaggctcgagttacacg

SEQ ID NO: 51 - DNA, 50 bp, Canis lupus
  source 1..50: mol_type = genomic DNA
cgttgtgtggtgtgcagggacactctgtgatactctagtgagctgctaag

SEQ ID NO: 52 - DNA, 50 bp, Canis lupus
  source 1..50: mol_type = genomic DNA
cgcctctgctcagtcctcaagccctgggcgtggtgcccagaatagggtgc

SEQ ID NO: 53 - DNA, 50 bp, Canis lupus
  source 1..50: mol_type = genomic DNA
cgctgttgcccttgggagccatggaaagggccaatttgctcgcagtctta

SEQ ID NO: 54 - DNA, 50 bp, Canis lupus
  source 1..50: mol_type = genomic DNA
atcttgttcagccatgtgtcgcctgccacaaactgcaaaacagaacaacg

SEQ ID NO: 55 - DNA, 50 bp, Canis lupus
  source 1..50: mol_type = genomic DNA
cgtaatgtaactgaaaattattgaagtgagaaaaaagagaaacagggaag

SEQ ID NO: 56 - DNA, 50 bp, Canis lupus
  source 1..50: mol_type = genomic DNA
gtcaatgtcagcaaatgtccacttactggattgaaggttgtttctaaacg

SEQ ID NO: 57 - DNA, 50 bp, Canis lupus
  source 1..50: mol_type = genomic DNA
ccagtcctgtctcctctgagggcatcaaggacttcttcagcatgaagccg

SEQ ID NO: 58 - DNA, 50 bp, Canis lupus
  source 1..50: mol_type = genomic DNA
cgagccccgccggccatcctcgctgatggtgggcatcacctcgcacatca

SEQ ID NO: 59 - DNA, 50 bp, Canis lupus
  source 1..50: mol_type = genomic DNA
cgccagcttgtccacctcgcgctccagctccgacttctgcttctgcagct

SEQ ID NO: 60 - DNA, 50 bp, Canis lupus
  source 1..50: mol_type = genomic DNA
cgattaggcagaaatgaaggagatcataaaggtgagagatttctccacaa

SEQ ID NO: 61 - DNA, 50 bp, Canis lupus
  source 1..50: mol_type = genomic DNA
cgtaaaggtcaagcattgtgacatacctttcaaataatcccgcctaactt

SEQ ID NO: 62 - DNA, 50 bp, Canis lupus
  source 1..50: mol_type = genomic DNA
cgagataatcttttaaggtgcactgttaaggtggaacctaaatgttgctg

SEQ ID NO: 63 - DNA, 50 bp, Canis lupus
  source 1..50: mol_type = genomic DNA
cgggaaagtggctttaataccctgaaaagcaaaggaatccgcctgtcagc

SEQ ID NO: 64 - DNA, 50 bp, Canis lupus
  source 1..50: mol_type = genomic DNA
ttgtggggaatcgtggccaagtgcactgactctgtgggaaaacggcggcg

SEQ ID NO: 65 - DNA, 50 bp, Canis lupus
  source 1..50: mol_type = genomic DNA
cgctggcgtagccgaagctggagtgctgctttgctttcagtctcaggctg

SEQ ID NO: 66 - DNA, 50 bp, Canis lupus
  source 1..50: mol_type = genomic DNA
cgctgggtaatggcccctgtaaagtgttaattgcctattgagactgcaga

SEQ ID NO: 67 - DNA, 50 bp, Canis lupus
  source 1..50: mol_type = genomic DNA
taatcaatcttgcagctgtcaggccgacaggcaggagtattaacctggcg

SEQ ID NO: 68 - DNA, 50 bp, Canis lupus
  source 1..50: mol_type = genomic DNA
cggctttttgaatgagccacgtgttcaaactacaaatcaacgtctcacgt

SEQ ID NO: 69 - DNA, 50 bp, Canis lupus
  source 1..50: mol_type = genomic DNA
aatatggctcgtccttcaatttgatgcttgagcaacagagggtcagagcg

SEQ ID NO: 70 - DNA, 50 bp, Canis lupus
  source 1..50: mol_type = genomic DNA
ggcacaccagctgcctgttttgcatggtatttgcaaaaatgcctcttgcg

SEQ ID NO: 71 - DNA, 50 bp, Canis lupus
  source 1..50: mol_type = genomic DNA
acaatagatctgagcagacagatgaaatttaaaacttcagcatgagtgcg

SEQ ID NO: 72 - DNA, 50 bp, Canis lupus
  source 1..50: mol_type = genomic DNA
cgtattttctgacaatatggaagaattcaaggatgatgtaatttcctctt

SEQ ID NO: 73 - DNA, 50 bp, Canis lupus
  source 1..50: mol_type = genomic DNA
gggggatcgagcgtttgggggcgctcgacttgtgccgccacttcttctcg

SEQ ID NO: 74 - DNA, 50 bp, Canis lupus
  source 1..50: mol_type = genomic DNA
caaattatagagttcagcttcaacacacgctctgtcacccgagatcagcg

SEQ ID NO: 75 - DNA, 50 bp, Canis lupus
  source 1..50: mol_type = genomic DNA
cgcgcccctctgcaggactgtgatttgttgtgtattagtacatctggcta

SEQ ID NO: 76 - DNA, 50 bp, Canis lupus
  source 1..50: mol_type = genomic DNA
tcttctgctcgtgcagtgcactctgggccttgagagcagagtcccgggcg

SEQ ID NO: 77 - DNA, 50 bp, Canis lupus
  source 1..50: mol_type = genomic DNA
tactgtactaattatgtaaattaaacctaattaactgacgaaaactgccg

SEQ ID NO: 78 - DNA, 50 bp, Canis lupus
  source 1..50: mol_type = genomic DNA
cggtctaccagaaagctagcccagtttagtgctcagtttcaaatgcatag

SEQ ID NO: 79 - DNA, 50 bp, Canis lupus
  source 1..50: mol_type = genomic DNA
cgttggagagcaactaaaatctgactgatttccatctttggagcatcaga

SEQ ID NO: 80 - DNA, 50 bp, Canis lupus
  source 1..50: mol_type = genomic DNA
ttgtggtgtaatcaacttgccatacatgcattacctcctaatgagcgccg

SEQ ID NO: 81 - DNA, 50 bp, Canis lupus
  source 1..50: mol_type = genomic DNA
cgtgaaagaaaatatgaatctaatttaaattcaaactggatttgggatat

SEQ ID NO: 82 - DNA, 50 bp, Canis lupus
  source 1..50: mol_type = genomic DNA
gcattactcgcagtcagctaaatgaaacattattctaaacatatgcatcg

SEQ ID NO: 83 - DNA, 50 bp, Canis lupus
  source 1..50: mol_type = genomic DNA
acacaatggcaggttccttgacaatgtcatgcgtctattcaaagcaaccg

SEQ ID NO: 84 - DNA, 50 bp, Canis lupus
  source 1..50: mol_type = genomic DNA
cgcctgcgcagacccaaatcttggtcccgccgtaaggtgccgcagtcccg

SEQ ID NO: 85 - DNA, 50 bp, Canis lupus
  source 1..50: mol_type = genomic DNA
cgttagtaatggaaaataccaggttgtttaaaattataataataattgtt

SEQ ID NO: 86 - DNA, 50 bp, Canis lupus
  source 1..50: mol_type = genomic DNA
cgccctgcatgtgtctgggctcccctggccagtccgtttttttgtctctg

SEQ ID NO: 87 - DNA, 50 bp, Canis lupus
  source 1..50: mol_type = genomic DNA
ttgtctcaaatcagctacctggtggacaatttaaccaagaaaaattaccg

SEQ ID NO: 88 - DNA, 50 bp, Canis lupus
  source 1..50: mol_type = genomic DNA
ttggaatcacaaagtggcccatggcggagaatgcagccagaacaaaggcg

SEQ ID NO: 89 - DNA, 50 bp, Canis lupus
  source 1..50: mol_type = genomic DNA
cgtcgggatgtttggctgtaatgccccaagatttgttctccctgaaaaaa

SEQ ID NO: 90 - DNA, 50 bp, Canis lupus
  source 1..50: mol_type = genomic DNA
cgagccctgctttcagtaatttgctgtaaactcaggggaggctggcgcta

SEQ ID NO: 91 - DNA, 50 bp, Canis lupus
  source 1..50: mol_type = genomic DNA
ccgctgacttctctgatcaacacataattatctctgaataaaaatgcacg

SEQ ID NO: 92 - DNA, 50 bp, Canis lupus
  source 1..50: mol_type = genomic DNA
cgagaaccgaacttaccaagcatcctctgcggctttcagtaaatacagcc

SEQ ID NO: 93 - DNA, 50 bp, Canis lupus
  source 1..50: mol_type = genomic DNA
attccaacactgaaacagcaccattccaaaagtggtaactagagaaaccg

SEQ ID NO: 94 - DNA, 50 bp, Canis lupus
  source 1..50: mol_type = genomic DNA
gccacacagagaagatagccgctgacggacacttcattttaattgtaacg

SEQ ID NO: 95 - DNA, 50 bp, Canis lupus
  source 1..50: mol_type = genomic DNA
cgcgtccaaggctgctgcttaatccaatgaaggcaatttccgaggataat

SEQ ID NO: 96 - DNA, 50 bp, Canis lupus
  source 1..50: mol_type = genomic DNA
aataaataatattctgcacatcaaatcactttcaccggcccccacccccg

SEQ ID NO: 97 - DNA, 50 bp, Canis lupus
  source 1..50: mol_type = genomic DNA
cgtggatgggaatttctaatagatctgcctggccctgtgctgcttttcaa

SEQ ID NO: 98 - DNA, 50 bp, Canis lupus
  source 1..50: mol_type = genomic DNA
catagtttacagctgtatccgctttccacacgtggcaaatgattgcctcg

SEQ ID NO: 99 - DNA, 50 bp, Canis lupus
  source 1..50: mol_type = genomic DNA
ggccttcatcgtgtgctggacgcctttcttcttcgtgcagatgtggagcg

SEQ ID NO: 100 - DNA, 50 bp, Canis lupus
  source 1..50: mol_type = genomic DNA
agccctactgtgttggaatagagcgtaaccagctggaggactgtaagacg

SEQ ID NO: 101 - DNA, 50 bp, Canis lupus
  source 1..50: mol_type = genomic DNA
cacccacaacacaagatcagaccccaagttctgctgtttcagttgccacg

SEQ ID NO: 102 - DNA, 50 bp, Canis lupus
  source 1..50: mol_type = genomic DNA
caccccatggaccagaaactcaaaaagtttgcttttcgtggctttgcgcg

SEQ ID NO: 103 - DNA, 50 bp, Canis lupus
  source 1..50: mol_type = genomic DNA
cggctttgttgccaatttcatgccatgtattgctccatgttttgtgcttc

SEQ ID NO: 104 - DNA, 50 bp, Canis lupus
  source 1..50: mol_type = genomic DNA
cgatgcacatctgccactgctgcaacacctcctcgtgctactggggctgc

SEQ ID NO: 105 - DNA, 50 bp, Canis lupus
  source 1..50: mol_type = genomic DNA
cgaaagtattgtgttccagctgcaggtcagggccgccaaagcttacctcc

SEQ ID NO: 106 - DNA, 50 bp, Canis lupus
  source 1..50: mol_type = genomic DNA
tgggcggggtagcccaacatgtggacgtagagcagtttggccagctgccg

SEQ ID NO: 107 - DNA, 50 bp, Canis lupus
  source 1..50: mol_type = genomic DNA
ccgtgctgattggtttcatccattttattgtcaaggaaattaacagcccg

SEQ ID NO: 108 - DNA, 50 bp, Canis lupus
  source 1..50: mol_type = genomic DNA
ctatgcccagaaactgaagtacaaggccattagcgaggagctggaccacg

SEQ ID NO: 109 - DNA, 50 bp, Canis lupus
  source 1..50: mol_type = genomic DNA
cgtcccctgtaacgtttccagcggcaaaacaaagagacgtctccagcaac

SEQ ID NO: 110 - DNA, 50 bp, Canis lupus
  source 1..50: mol_type = genomic DNA
cgctggggtctgccccttgggcacgtcaaagcttcagacctgacaaatca

SEQ ID NO: 111 - DNA, 50 bp, Canis lupus
  source 1..50: mol_type = genomic DNA
cgactcacagactggaacatttctgtgatccgctgtaatgcactggggga

SEQ ID NO: 112 - DNA, 50 bp, Canis lupus
  source 1..50: mol_type = genomic DNA
tgtgatccccactatctcaagcatcgtcccggagagctgcctgctgatcg

SEQ ID NO: 113 - DNA, 50 bp, Canis lupus
  source 1..50: mol_type = genomic DNA
tgacctctgcatgatcccggactctatgaattattgatgagatatgagcg

SEQ ID NO: 114 - DNA, 50 bp, Canis lupus
  source 1..50: mol_type = genomic DNA
cgtatgcaaaaggcacaattattcacccaccaaggtgacagagaaggcct

SEQ ID NO: 115 - DNA, 50 bp, Canis lupus
  source 1..50: mol_type = genomic DNA
attgtaaattacctgtgacatctcattaatcctctttttcctcacaaacg

SEQ ID NO: 116 - DNA, 50 bp, Canis lupus
  source 1..50: mol_type = genomic DNA
agctgatttcttcactccagcaaaagcactttaattcccttttagatacg

SEQ ID NO: 117 - DNA, 50 bp, Canis lupus
  source 1..50: mol_type = genomic DNA
attatccctctaccttaccacccaccagtgttgtggatttaagagagtcg

SEQ ID NO: 118 - DNA, 50 bp, Canis lupus
  source 1..50: mol_type = genomic DNA
ttaaaaccagttctatccactgtaacaatgacctggagccaaacaaggcg

SEQ ID NO: 119 - DNA, 50 bp, Canis lupus
  source 1..50: mol_type = genomic DNA
cggctgcattccgactcacagactggaacatttctgtgatccgctgtaat

SEQ ID NO: 120 - DNA, 50 bp, Canis lupus
  source 1..50: mol_type = genomic DNA
cgtctgtggcggtttttctataggtgctaaaatattccaccctggtgact

SEQ ID NO: 121 - DNA, 50 bp, Canis lupus
  source 1..50: mol_type = genomic DNA
atgtaatgttcggcagcaggaggtaaattcctctccaatttccaggcccg

SEQ ID NO: 122 - DNA, 50 bp, Canis lupus
  source 1..50: mol_type = genomic DNA
gataaattccagtccacagacgccttataaattacatttttttgttcgcg

SEQ ID NO: 123 - DNA, 50 bp, Canis lupus
  source 1..50: mol_type = genomic DNA
cgagttagacaggtgattagcataattagcaccgagcagctatacatatg

SEQ ID NO: 124 - DNA, 50 bp, Canis lupus
  source 1..50: mol_type = genomic DNA
cggatccgctccgactccagggtgatctgctcaaaggctgagtcacacac

SEQ ID NO: 125 - DNA, 50 bp, Canis lupus
  source 1..50: mol_type = genomic DNA
agcaccagtacaggtcggtgacggcgatgaggtacaggtccagcaggccg

SEQ ID NO: 126 - DNA, 50 bp, Canis lupus
  source 1..50: mol_type = genomic DNA
gctcagctccattggaatgctccgggcgctgtccaaggtgctggaatgcg

SEQ ID NO: 127 - DNA, 50 bp, Canis lupus
  source 1..50: mol_type = genomic DNA
agacacccacctgtatgagtacgcttgtggatcttgaggttctcggagcg

SEQ ID NO: 128 - DNA, 50 bp, Canis lupus
  source 1..50: mol_type = genomic DNA
aactggacagcaccatgtccaccaaagcggagcagtgtaagtagcagccg

SEQ ID NO: 129 - DNA, 50 bp, Canis lupus
  source 1..50: mol_type = genomic DNA
actgaccaatggcagaggcaggaattgtcaaatagcacccaggaggagcg

SEQ ID NO: 130 - DNA, 50 bp, Canis lupus
  source 1..50: mol_type = genomic DNA
cggtgatttacttccctgcaaatgagttgtttcatattttgcactgtctt

SEQ ID NO: 131 - DNA, 50 bp, Canis lupus
  source 1..50: mol_type = genomic DNA
cggactctacctgtggctcaggcataccaggacaacctgtacaggcagct

SEQ ID NO: 132 - DNA, 50 bp, Canis lupus
  source 1..50: mol_type = genomic DNA
cgctccccctctaatgtgtgatctggaagctctataaagcctgatgtaat

SEQ ID NO: 133 - DNA, 50 bp, Canis lupus
  source 1..50: mol_type = genomic DNA
cggcttgatatttccgaagaatatagtgggctttattagcaccagtttcg

SEQ ID NO: 134 - DNA, 50 bp, Canis lupus
  source 1..50: mol_type = genomic DNA
cgctgaaccaggagacaaacacgattaccagctccgagccttgagtcaga

SEQ ID NO: 135 - DNA, 50 bp, Canis lupus
  source 1..50: mol_type = genomic DNA
cgatcagatgagttcctcttagtgcaggtcaaaaaggctagttagcagga

SEQ ID NO: 136 - DNA, 50 bp, Canis lupus
  source 1..50: mol_type = genomic DNA
ttaagcacatttacatttggttcttaaaatccaaaatagcccatttcacg

SEQ ID NO: 137 - DNA, 50 bp, Canis lupus
  source 1..50: mol_type = genomic DNA
cgtcttcttcaactggctgggctacgccaactcggccttcaaccccatca

SEQ ID NO: 138 - DNA, 50 bp, Canis lupus
  source 1..50: mol_type = genomic DNA
cgcaccgcactccatatcgaggatggattgttttatgctgatgcaatgtg

SEQ ID NO: 139 - DNA, 50 bp, Canis lupus
  source 1..50: mol_type = genomic DNA
agatctcgcctttccagatgcaaaagttcagcccctctgatgtcatgacg

SEQ ID NO: 140 - DNA, 50 bp, Canis lupus
  source 1..50: mol_type = genomic DNA
cgaatgcagctgctctttgtaattgtttgtgaaactgagttaaagggagg

SEQ ID NO: 141 - DNA, 50 bp, Canis lupus
  source 1..50: mol_type = genomic DNA
agaaggaccttgtaataaataatattctgcacatcaaatcactttcaccg

SEQ ID NO: 142 - DNA, 50 bp, Canis lupus
  source 1..50: mol_type = genomic DNA
ctcctaggagctcacagctccaaacatcaattaccatgattatctacccg

SEQ ID NO: 143 - DNA, 50 bp, Canis lupus
  source 1..50: mol_type = genomic DNA
ttaactgtgaaattttatttccgttaaaaagcaagcctgtaatcaaaacg

SEQ ID NO: 144 - DNA, 50 bp, Canis lupus
  source 1..50: mol_type = genomic DNA
cgctcccccgccgagctggggtagctgatcactgagctgaaactaaacgt

SEQ ID NO: 145 - DNA, 50 bp, Canis lupus
  source 1..50: mol_type = genomic DNA
acgtgtggcccagcaggttgggcatgcgggtcaggttgtagccgatgccg

SEQ ID NO: 146 - DNA, 50 bp, Canis lupus
  source 1..50: mol_type = genomic DNA
cgccttcctcatcggctgcatgttcatcaagatgtcccagcccaagaagc

SEQ ID NO: 147 - DNA, 50 bp, Canis lupus
  source 1..50: mol_type = genomic DNA
cgctggagctcctacatggtgcactggaagaaccagttcgaccactacag

SEQ ID NO: 148 - DNA, 50 bp, Canis lupus
  source 1..50: mol_type = genomic DNA
cgctctcttgacagctcgattgcgtgctgcctctgctcctgcataaatca

SEQ ID NO: 149 - DNA, 50 bp, Canis lupus
  source 1..50: mol_type = genomic DNA
cgccatcatcaacgtggtggtcttcatccagccctactgggtgggcgaca

(19) United States
(12) Patent Application Publication
(10) Pub. No.: US 2025/0182905 A1
(43) Pub. Date: Jun. 5, 2025

(54) METHOD

(71) Applicant: SOCIETE DES PRODUITS NESTLE S.A., VEVEY (CH)

(72) Inventors: Alix ZOLLINGER, Denges (CH); Lorane TEXARI, Morges (CH); Philipp GUT, Genève (CH); Sébastien HERZIG, Morges (CH)

(21) Appl. No.: 18/961,661

(22) Filed: Nov. 27, 2024

Related U.S. Application Data
(60) Provisional application No. 63/604,380, filed on Nov. 30, 2023.

(51) Int. Cl. G16H 50/30; C12Q 1/6806; C12Q 1/6827; C12Q 1/6874; G16B 20/20; G16B 40/20; G16H 20/10; G16H 20/60

(52) U.S. Cl. CPC G16H 50/30; C12Q 1/6806; C12Q 1/6827; C12Q 1/6874; G16B 20/20; G16B 40/20; G16H 20/10; G16H 20/60; C12Q 2600/156

(57) ABSTRACT

A method for determining a mortality risk and/or probability of a healthy lifespan of a dog, the method including a) providing a DNA methylation profile from a sample obtained from the dog; and b) determining a mortality risk and/or probability of a healthy lifespan for the dog using the DNA methylation profile. The DNA methylation profile contains at least one methylation site as listed in Table 3.

### SEQUENCE LISTING

The instant application contains a Sequence Listing which has been submitted electronically in XML file format and is hereby incorporated by reference in its entirety. Said XML copy, created on Dec. 4, 2024, is named 3714652-00006_SL.xml and is 135,645 bytes in size.

### PRIORITY CLAIM

The present application claims priority to U.S. Provisional Patent Application No. 63/604,380 filed Nov. 30, 2023, the disclosure of which is incorporated herein by reference for all purposes.

### FIELD OF THE INVENTION

The present invention relates to a method for determining the health status of a dog using a DNA methylation profile. In particular the invention relates to methods of selecting a lifestyle regime, dietary regime or therapeutic intervention for the dog, or determining the efficacy of a lifestyle regime, dietary regime or therapeutic intervention, based on the health status determined from the DNA methylation profile.

### BACKGROUND

The ability to determine information regarding the health of a dog is desirable to inform about the dog's general health and well-being.

Chronological age is known to be a major indicator of general health status, with increasing chronological age associated with reduced health. However, depending on genetics, nutrition, and lifestyles, individuals may age slower or faster than their chronological age. Chronological age may therefore not always reflect an individual's rate of aging or risk of reduced health. On the other hand, the biological age of an individual (based on e.g. clinical biochemistry and cell biology measures) can vary compared to others of the same chronological age. Methods for determining biological age may be helpful for identifying individuals at risk of age-related disorders earlier than would be expected based on their chronological age (see e.g. WO2019/046725).

Epigenetic clocks for predicting chronological age and inferring health states as an indicator of biological age are described in WO2022/272120. These epigenetic clocks are primarily based on chronological age as the training parameter.

However, there is a need for further methods of determining the biological age of a dog and utilising measures of biological age to improve health outcomes for a dog.

### SUMMARY

The present invention relates to a method for quantifying the health status of a dog based on a DNA methylation profile. The method enables a determination of mortality risk and/or probability of a healthy lifespan for a dog through assessment of a DNA methylation profile from the dog.

Existing methods that assess the health status of dogs determine biological age based on correlations between DNA methylation and chronological age (see e.g. WO2022/272120). Calculating the biological age of an animal may comprise determining a DNA methylation profile compared to an expected DNA methylation profile at a given chronological age. Such methods are therefore based on the use of chronological age as the primary indicator of overall health.

In contrast, the present invention takes into account the direct predictive value of the DNA methylation profile on mortality risk and/or probability of a healthy lifespan. By way of example, a given DNA methylation marker may not directly correlate with chronological age, but may be indicative of a particular pathological condition and thus an increased mortality risk and/or a probability of a reduced healthy lifespan. The present methods may thus be described as identifying the mortality risk and/or a probability of a healthy lifespan of a dog. As such, the DNA methylation markers and DNA methylation profiles of the present invention do not necessarily correlate with chronological age, but are related to the difference between phenotypic and chronological age of the dog.

In a first aspect, the present invention provides a method for determining a mortality risk of a dog; said method comprising: a) providing a DNA methylation profile from a sample obtained from the dog; and b) determining a mortality risk for the dog using the DNA methylation profile; wherein the DNA methylation profile comprises at least one methylation site as listed in Table 3.

Advantageously, the present methods may be performed using commercially available DNA methylation arrays (e.g. available from Illumina).

Determining a mortality risk may refer to determining a likelihood that a dog will live for a longer or shorter period of time compared to an equivalent dog of—for example—the same chronological age, sex and breed. Accordingly, the present methods may determine the probability of a lifespan, health span and/or longevity for a dog compared to an equivalent dog of—for example—the same chronological age, sex and breed. In addition, methods for improving the mortality risk and/or probability of a healthy lifespan for the dog may improve the probable lifespan, health span and/or longevity of the dog.

As used herein, ‘lifespan’ may refer to the length of time (e.g. years) for which a subject lives. ‘Health span’ may refer to length of time (e.g. years) of life without disease. ‘Longevity’ may refer to length of time (e.g. years) that a subject lives beyond its expected lifespan.

Suitably, mortality risk may be equated to the probability of a healthy lifespan for the dog; wherein a decreased mortality risk is equated to an increased probably of longer healthy lifespan for the dog or an increased mortality risk is equated to a decreased probability of longer healthy lifespan for the dog. The mortality risk may be represented as the difference between determined age (i.e. biological age) and chronological age of the dog. For example, an increase in the difference between the biological age determined by the present method compared to chronological age may be indicative of an increased mortality risk for the dog. A decrease in the difference between the biological age determined by the present method compared to chronological age may be indicative of a decreased mortality risk for the dog. Suitably, the mortality risk and/or a probability of a healthy lifespan may be described as the biological age of the dog. Suitably, the mortality risk and/or a probability of a healthy lifespan may be described as the epigenetic age of the dog. Suitably, the present biological clock may be referred to as an epigenetic clock.

Suitably, determining that the biological age of the dog is greater than its chronological age is indicative of a higher mortality risk. Suitably, determining that the biological age of the dog is less than its chronological age is indicative of a reduced mortality risk. Suitably, determining that the biological age of the dog is greater than its chronological age is indicative of a reduced probability of a longer healthy lifespan. Suitably, determining that the biological age of the dog is less than its chronological age is indicative of an increased probability of a longer healthy lifespan.

Suitably, the present methods may be used to determine a biological age for a dog based on its mortality risk and/or probability of a healthy lifespan.

Accordingly, in a further aspect the invention provides a method for determining a biological age of a dog; said method comprising: a) providing a DNA methylation profile from a sample obtained from the dog; and b) determining a biological age for the dog using the DNA methylation profile, wherein the DNA methylation profile is linked to the mortality risk and/or probability of a healthy lifespan for the dog and wherein the DNA methylation profile comprises at least one methylation site as listed in Table 3.

In all the present methods, determining or improving a mortality risk and/or probability of a healthy lifespan of a dog, also applies to determining or improving a biological age of a dog; wherein the biological age of the dog is determined using a DNA methylation profile that is linked to the mortality risk and/or probability of a healthy lifespan for the dog and wherein the DNA methylation profile comprises at least one methylation site as listed in Table 3.

In a further aspect, the invention provides a method for selecting a lifestyle regime, dietary regime or therapeutic intervention for a dog, the method comprising: a) providing a DNA methylation profile from a sample obtained from the dog; b) determining a mortality risk and/or probability of a healthy lifespan for the dog using the DNA methylation profile, wherein the DNA methylation profile comprises at least one methylation site as listed in Table 3; and c) selecting a suitable lifestyle regime, dietary regime or therapeutic intervention for the dog based on the mortality risk determined in step b).

As used herein, ‘selecting a suitable lifestyle regime, dietary regime or therapeutic intervention for a dog’ may also encompass ‘recommending a lifestyle regime, dietary regime or therapeutic intervention for the dog’ or ‘providing a recommended lifestyle regime, dietary regime or therapeutic intervention for the dog’.

In another aspect, the invention provides a method for determining the efficacy of a lifestyle regime, dietary regime or therapeutic intervention for improving the mortality risk and/or probability of a healthy lifespan of a dog, said method comprising: a) applying a lifestyle regime, dietary regime or therapeutic intervention to the dog, wherein the lifestyle regime, dietary regime or therapeutic intervention has been selecting according to the previous aspect of the invention; b) after a time period of applying the lifestyle regime, dietary regime or therapeutic intervention to the dog; determining a mortality risk and/or probability of a healthy lifespan of the dog using a DNA methylation profile from a sample obtained from the dog, wherein the DNA methylation profile comprises at least one methylation site as listed in Table 3; c) determining if there has been a change in the mortality risk and/or probability of a healthy lifespan of the dog after the time period of following the lifestyle regime, dietary regime or therapeutic intervention.

In a further aspect, the present invention provides a method for determining the efficacy of a lifestyle regime, dietary regime or therapeutic intervention for improving the mortality risk and/or probability of a healthy lifespan of a dog, said method comprising: a) determining a mortality risk for the dog using a DNA methylation profile from a sample obtained from the dog, wherein the DNA methylation profile comprises at least one methylation site as listed in Table 3; b) applying a lifestyle regime, dietary regime or therapeutic intervention selected based on the mortality risk determined in step a) to the dog; c) after a time period of applying a lifestyle regime, dietary regime or therapeutic intervention to the dog; determining a mortality risk of the dog using a DNA methylation profile from a sample obtained from the dog, wherein the DNA methylation profile comprises at least one methylation site as listed in Table 3; d) determining if there has been a change in the mortality risk of the dog between step a) and step c).

In a further aspect, the present invention provides a method for determining the efficacy of a lifestyle regime, dietary regime or therapeutic intervention for improving the mortality risk and/or probability of a healthy lifespan of a dog, said method comprising: a) determining a mortality risk for the dog using a DNA methylation profile from a sample obtained from the dog, wherein the DNA methylation profile comprises at least one methylation site as listed in Table 3; b) applying a lifestyle regime, dietary regime or therapeutic intervention selected based on the mortality risk determined in step a) to the dog; c) after a time period of applying a lifestyle regime, dietary regime or therapeutic intervention to the dog; determining a mortality risk of the dog using a DNA methylation profile from a sample obtained from the dog; d) determining if there has been a change in the mortality risk of the dog between step a) and step c).

In a further aspect, the present invention provides a method for determining the efficacy of a lifestyle regime, dietary regime or therapeutic intervention for improving the mortality risk and/or probability of a healthy lifespan of a dog, said method comprising: a) determining a mortality risk for the dog using a DNA methylation profile from a sample obtained from the dog; b) applying a lifestyle regime, dietary regime or therapeutic intervention selected based on the mortality risk determined in step a) to the dog; c) after a time period of applying a lifestyle regime, dietary regime or therapeutic intervention to the dog; determining a mortality risk of the dog using a DNA methylation profile from a sample obtained from the dog, wherein the DNA methylation profile comprises at least one methylation site as listed in Table 3; d) determining if there has been a change in the mortality risk of the dog between step a) and step c).

Suitably, improving the mortality risk and/or probability of a healthy lifespan of a dog may refer to a reduction in the difference between the biological age and chronological age of the dog, where the biological age of the dog is greater than its chronological age. Further, improving the mortality risk and/or probability of a healthy lifespan of a dog may refer to maintaining or further increasing the difference between the biological age and chronological age of the dog, where the biological age of the dog is less than its chronological age. Alternatively, a worsening in the mortality risk and/or probability of a healthy lifespan of a dog may refer to an increase in the difference between the biological age and chronological age of the dog, where the biological age of the dog is greater than its chronological age. A worsening in the mortality risk and/or probability of a healthy lifespan of a dog may also refer to a decrease in the difference between the biological age and chronological age of the dog, where the biological age of the dog is less than its chronological age.

Suitably, improving the mortality risk and/or probability of a healthy lifespan of a dog may refer to a reduction in the rate of change between the biological age and chronological age of the dog, where the biological age of the dog is greater than its chronological age. For example, a dog's biological age may have been increasing by 1.5 years per 1 year increase in chronological age. Following a lifestyle and dietary regime intervention, a reduction in the rate of change such that the dog's biological age subsequently increases by 1.25 years per 1 year increase in chronological age may provide an improvement in the dog's mortality risk and/or probability of a healthy lifespan.

Improving the mortality risk and/or probability of a healthy lifespan may also refer to maintaining or increasing in the rate of change between the biological age and chronological age of the dog, where the biological age of the dog is less than its chronological age. For example, a dog's biological age may have been increasing by less than 1 year (e.g 0.9 years) per 1 year increase in chronological age. Following a lifestyle, dietary regime or therapeutic intervention, the rate of change may alter such that the dog's biological age subsequently increases by, for example, 0.8 years or fewer per 1 year increase in chronological age may provide an improvement in the dog's biological age.

The present methods for determining the efficacy of a lifestyle regime, dietary regime or therapeutic intervention for improving the mortality risk and/or probability of a healthy lifespan of a dog may advantageously allow ongoing monitoring of the effectiveness of a lifestyle regime, dietary regime or therapeutic intervention for improving or maintaining the health of the dog. The use of such methods may advantageously allow particularly effective lifestyle regime, dietary regime or therapeutic interventions to be identified. In contrast, if a lifestyle regime, dietary regime or therapeutic intervention is determined to be ineffective based on the morality risk and/or probability of a healthy lifespan of the dog; an alternative lifestyle regime, dietary regime or therapeutic intervention may then be implemented.

Accordingly, the present method enables a suitable lifestyle regime, dietary regime or therapeutic intervention to be selected for the dog, based on its mortality risk and/or probability of a healthy lifespan as determined from the DNA methylation profile. For example, highly digestible and high-quality protein diets are generally recommended based upon the chronological age of a dog. For example, it may be recommended that a dog is switched to a senior diet around 7 or 8 years old. However, in the context of the present invention, the determination of an increased mortality risk and/or reduced probability of a healthy lifespan (i.e. an increased biological age) for a dog compared to its chronological age may allow a determination to switch the dog to a senior diet at an earlier age. In contrast, a dog with a reduced mortality risk and/or increased probability of a healthy lifespan (i.e. reduced biological age) compared to its chronological age may be able to stay on an adult diet for longer.

Suitably, the present methods may comprise selecting and/or applying a lifestyle regime, dietary regime or therapeutic intervention to a dog following a determination that the dog has an increased mortality risk and/or decreased probability of a healthy lifespan compared to its chronological age.

In another aspect, the invention provides a method for preventing or reducing the risk of a dog developing a disease; the method comprising:
a) determining a mortality risk and/or probability of a healthy lifespan of the dog using a DNA methylation profile from a sample obtained from the dog wherein the DNA methylation profile comprises at least one methylation site as listed in Table 3 and wherein the mortality risk and/or probability of a healthy lifespan determined for the dog is associated with an increased likelihood to develop the disease; and
b) selecting a lifestyle regime, dietary regime or therapeutic intervention for the dog based on the mortality risk and/or probability of a healthy lifespan determined in step a);
wherein the lifestyle regime, dietary regime or therapeutic intervention prevents or reduces the risk of the dog developing the disease.

Suitably, the disease is an age-related disease. For example, the age-related disease osteoarthritis, dementia, cognitive dysfunction, pre-diabetic condition, diabetes, cancer, heart disease, obesity, gastrointestinal disorders, incontinence, kidney disease, sarcopenia, vision loss, hearing loss, osteoporosis, cataracts, cerebrovascular disease, and/or liver disease.

The method may optionally further comprise administering the lifestyle regime, dietary regime or therapeutic intervention to the dog. Suitably, the lifestyle regime may be a dietary intervention or a therapeutic modality.

In another aspect, the invention provides a method for selecting a dog as being suitable for receiving an anti-aging lifestyle regime, dietary regime or therapeutic intervention; the method comprising: a) determining a mortality risk and/or probability of a healthy lifespan of the dog using a DNA methylation profile from a sample obtained from the dog wherein the DNA methylation profile comprises at least one methylation site as listed in Table 3; and b) selecting a dog as being suitable for receiving an anti-aging lifestyle regime, dietary regime or therapeutic intervention if it has an increased mortality risk and/or reduced probability of a healthy lifespan compared to its chronological age.

Suitably, whilst an anti-aging lifestyle regime, dietary regime or therapeutic intervention may be effective for dogs based on chronological age, it may be particularly effective when applied to a dog with an increased mortality risk and/or decreased probability of a healthy lifespan compared to its chronological age. As such, the present method may advantageously enable the selection of a dog that has an increased likelihood to respond, or improved magnitude of response, to the anti-aging lifestyle regime, dietary regime or therapeutic intervention.

The lifestyle regime, dietary regime or therapeutic intervention may be selected based on a determination that the dog has an increased mortality risk and/or reduced probability of a healthy lifespan (i.e. increased biological age) compared to its chronological age.

The lifestyle regime, dietary regime or therapeutic intervention may be a dietary intervention. The dietary intervention may be a calorie-restricted diet, a senior diet or a low protein diet.

The DNA methylation profile may be associated with increased biological age of (i) a tissue; (ii) an organ; or (iii) a physiological system, such as the immune, gastrointestinal, urinary, muscular, cardiovascular, and/or neurological system.

The invention further provides a dietary intervention for use in reducing the mortality risk and/or increasing the probability of a healthy lifespan of a dog, wherein the dietary intervention is administered to a dog with a mortality risk and/or probability of a healthy lifespan determined by the method of the invention.

The invention further relates to the use of a dietary intervention to reduce the mortality risk and/or increase the probability of a healthy lifespan of a dog, wherein the dietary intervention is administered to a dog with a mortality risk and/or probability of a healthy lifespan determined by the method of the invention.

In another aspect the invention provides a computer-readable medium comprising instructions that when executed cause one or more processors to perform the method of the invention.

In another aspect the invention provides a computer system for determining a mortality risk of a dog; the computer system programmed to determine a mortality risk for the dog using a DNA methylation profile of the dog.

In another aspect the invention provides a computer system for selecting a suitable lifestyle regime, dietary regime or therapeutic intervention for a dog, the computer system programmed to perform one or more of the steps of: a) determining a mortality risk for the dog using a DNA methylation profile from the dog wherein the DNA methylation profile comprises at least one methylation site as listed in Table 3; and b) selecting a suitable lifestyle regime, dietary regime or therapeutic intervention for the dog based on the mortality risk determined in step a).

In another aspect the invention provides a computer system for determining the efficacy of a lifestyle regime, dietary regime or therapeutic intervention for improving the mortality risk for a dog, the computer system programmed to perform one or more of the steps of: a) determining a mortality risk of the dog using a DNA methylation profile from a sample obtained from the dog before the lifestyle regime, dietary regime or therapeutic intervention and a sample obtained from the dog after the lifestyle regime, dietary regime or therapeutic intervention wherein the DNA methylation profile comprises at least one methylation site as listed in Table 3; and b) determining if there has been a change in the mortality risk of the dog between the sample obtained from the dog before and after the lifestyle regime, dietary regime or therapeutic intervention has been applied.

In another aspect the invention provides a computer system for determining a likelihood that a dog will benefit from an anti-aging lifestyle regime, dietary regime or therapeutic intervention; the computer system programmed to perform one or more of the steps of: a) determining a mortality risk for the dog using a DNA methylation profile from a sample obtained from the dog wherein the DNA methylation profile comprises at least one methylation site as listed in Table 3; b) identifying a dog as likely to respond to an anti-aging lifestyle regime, dietary regime or therapeutic intervention if it has an increased mortality risk compared to its chronological age.

In another aspect the invention provides a computer program product comprising computer implementable instructions for causing a programmable computer to determine a mortality risk for the dog using a DNA methylation profile of the dog; wherein the DNA methylation profile comprises at least one methylation site as listed in Table 3.

In another aspect the invention provides a computer program product comprising computer implementable instructions for causing a programmable computer to determine a mortality risk for the dog using a DNA methylation profile from the dog wherein the DNA methylation profile comprises at least one methylation site as listed in Table 3; and select a suitable lifestyle regime, dietary regime or therapeutic intervention for the dog based on the mortality risk determined using a DNA methylation profile.

In another aspect the invention provides a computer program product comprising computer implementable instructions for causing a programmable computer to a) determine a mortality risk of a dog using a DNA methylation profile from a sample obtained from the dog before a lifestyle regime, dietary regime or therapeutic intervention and a sample obtained from the dog after the lifestyle regime, dietary regime or therapeutic intervention wherein the DNA methylation profile comprises at least one methylation site as listed in Table 3; and b) determine if there has been a change in the mortality risk of the dog between the sample obtained from the dog before and after the lifestyle regime, dietary regime or therapeutic intervention has been applied.

In another aspect the invention provides a computer program product comprising computer implementable instructions for causing a programmable computer to a) determine a mortality risk for a dog using a DNA methylation profile from a sample obtained from the dog wherein the DNA methylation profile comprises at least one methylation site as listed in Table 3; and b) identify a dog as likely to respond to an anti-aging lifestyle regime, dietary regime or therapeutic intervention if it has an increased mortality risk compared to its chronological age.

Advantageously, the present invention may allow a mortality risk and/or probability of a healthy lifespan to be determined based on markers of multiple organ systems and functions. Accordingly, the present methods may advantageously encompassed a range of potential organ dysfunctions.

Evaluating the mortality risk and/or probability of a healthy lifespan of a dog allows one to test several aspects of the animal's wellbeing. First, it can predict whether this animal is more likely to need a dietary or supplement-based intervention. It can also be used to test the efficacy of a dietary or supplement-based intervention on aging.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1—Identification of blood biomarkers predictive of mortality risk.

A cox proportional hazard model was fit for each of the 28 biomarkers assessed, including sex and breed class (small or medium). Values are adjusted for the p. value of each parameter to account for multiple comparison (by false discovery rate (fdr)). Parameters show are those with an adjusted fdr below 0.05.

FIG. 2—Demonstration of biomarkers that contribute to the predictive ability of the multi-parameter model for determining phenoage.

FIG. 3—shows the correlation between phenoDNAmAge (biological age according to the present biological clock) and chronological age.

FIG. 4—The hazard ratio of a cox model explaining survival by sex and delta, stratified on breed class. Delta_res is obtained as the residuals of a linear model between phenoDNAmAge and chronological age.

FIG. 5—A validation data set based on a life long calorie restriction study.

FIG. 6—shows illustrative epigenetic clocks comprising the A) top 5, B) top 10, C) top 30, D) top 50 methylation sites from the full epigenetic clock correlate with chronological age

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by veterinarians, health-care professionals, lab technicians, pet care providers and so on.

### Subject

The present methods are directed to canine subjects. Accordingly, the subject of the present invention is a dog.

In an alternative aspect, the subject may be a feline subject. Accordingly, in the alternative aspects of the invention, the subject is a cat. All disclosures herein are equally applicable to a cat, unless stated otherwise.

### Breed

The present methods may utilise information regarding the breed of the dog. The dog may be categorised as a toy, small, medium, large or giant breed—for example. Suitably, the dog breed may be categorised based on the weight of the dog. Suitably, the dog breed may be categorised based on the average weight of a dog for a given breed.

Suitably, the dog may be categorised as a small or medium breed. Suitably, the categorisation is determined by the average weight of adult dogs of this breed. Suitably, a breed with an average weight below 10 kg is categorised as a small breed and/or a breed with an average weight above 10 kg is categorised as a medium breed.

In the alternative aspect where the subject is a cat, the cat may be a domestic cat. Suitably, the cat may be a Domestic Shorthair cat.

### Sex

Suitably, the sex of the dog may be classified as male or female.

### Chronological Age

Chronological age may be defined as the amount of time that has passed from the subject's birth to the given date. Chronological age may be expressed in terms of years, months, days, etc.

Suitably, the present method may be applied to a dog of any chronological age. In certain embodiments, the dog may be at least about 2 years old. Suitably, the dog may be at least about 2, at least about 3, at least about 4, at least about 5, at least about 6, at least about 7, at least about 8, at least about 9 or at least about 10 years old.

Suitably, the dog may be at least about 7 years old.

### Sample

The present invention comprises a step of providing or determining a DNA methylation profile from one or more samples obtained from a subject.

Suitably, the sample is a blood, hair follicle, buccal swab, saliva or tissue sample.

Suitably, the sample is a hair follicle, buccal swab or saliva sample. Such sample types are particularly applicable if the sample is to be provided, for example, outside of a veterinarian environment—for example using a kit according to the present invention.

Suitably, the sample is derived from blood. The sample may contain a blood fraction or may be whole blood. The sample preferably comprises whole blood. The sample may comprise a peripheral blood mononuclear cell (PBMC) or lymphocyte sample. Techniques for collecting samples from a subject and extracting DNA (e.g. genomic DNA) from the sample are well known in the art.

The present methods may be performed on one or more samples obtained from the subject. For example, the method may be performed using a first sample obtained at a given time point and a second sample obtained following a time interval after the first sample was obtained. The method may be performed more than once, on samples obtained from the same dog over a time period. For example, samples may be obtained repeatedly once per month, once a year, or once every two years. Suitably, the samples may be obtained around once per year (e.g. during an annual veterinary health check). This may be useful in determining the effects of a particular treatment or change in lifestyle-such as a dietary intervention or a change in exercise regime.

In one embodiment, the method may be applied to a sample obtained from a subject prior to a change in lifestyle (e.g. a dietary product intervention or a change in exercise regime). In another embodiment, the method may be applied to a sample obtained from a subject prior to, and after the e.g. dietary product intervention or change in exercise regime. The method may also be applied to samples obtained at predetermined times throughout the e.g. dietary product intervention or change in exercise regime. These predetermined times may be periodic throughout the e.g. dietary product intervention or change in exercise regime, e.g. every day or three days, or may depend on the subject being tested.

### DNA Methylation

DNA methylation is the process by which a methyl group (CH3) is added covalently to a cytosine base that is part of a DNA molecule. In vivo, this process is catalysed by a family of DNA methyltransferases (Dnmts), that generate the modified cytosine by transfer of a methyl group from S-adenyl methionine (SAM). The cytosine is modified on the 5th carbon atom, and the modified residue is known as 5-methylcytosine (5 mC). The DNA methylation may also comprise 5-hydroxymethylcytosine (5 hmc).

DNA methylation is an example of an epigenetic mechanism, i.e. it is capable of modifying gene expression without modification of the underlying DNA sequence. DNA methylation can, for example, inhibit the expression of genes by acting as a recruitment signal for repressive factors, or by directly blocking transcription factor recruitment. DNA methylation predominantly occurs in the genome of somatic mammalian cells at sites of adjacent cytosine and guanine that form a dinucleotide (CpG). While non-CpG methylation is observed in embryonic development, in the adult these modifications are much reduced in most cell types. CpG islands are stretches of DNA that have a high CpG density, but are generally unmethylated. These regions are associated with promoter regions, particularly promoter regions of housekeeping genes, and are thought to be maintained in a permissive state to allow gene expression.

DNA methylation has been found to vary with age in humans and other animals. Aged mammalian tissues show overall DNA hypomethylation, which is considered to be due to a gradual loss or mis-targeting of DMNT1 methyltransferase activity, but local hypermethylation of CpG islands. Local hypermethylation can result in repression of certain genes and this can contribute towards age-related disease. The link between epigenetic changes in DNA methylation with age allows the estimation of a “biological age” using “DNA methylation clocks”. Generally, these clocks have been trained against chronological age using supervised machine learning approaches, and deviations of the “clock age” from the actual chronological age for an individual is considered an indicator of “biological” age. This correlates with the chronological age of the individual, but deviations from correlation can indicate potential risk of age-related disease or illness in individuals.

The detection of specific methylated DNA can be accomplished by multiple methods (see e.g. Zuo et al., 2009; Epigenomics. 1 (2): 331-345) and Rauluseviciute et al.; Clinical Epigenetics; 2019; 11 (193)). A number of methods are available for detection of differentially methylated DNA at specific loci in samples such as blood, urine, stool or saliva. These methods are able to distinguish 5-methyl cytosine or methylated DNA from unmethylated DNA, and subsequently quantify the proportion of methylated and unmethylated DNA for a particular genomic site.

The present methods may comprise determining a DNA methylation profile for dog using any suitable method. Suitable methods include, but are not limited to, those described below.

### Enzymatic Methyl-seq (EM-seq)

Suitably, enzymatic approaches are used to detect 5 mC and 5 hmC. By way of example, Enzymatic Methyl-seq (EM-seq) may be used.

Typically in EM-seq, in a first enzymatic step, 5 mC is oxidized to 5 hmC, then 5 fC and finally 5 caC by the activity of Tet methylcytosine dioxygenase 2 (TET2). In addition, use of a T4-BGT enzyme glucosylates both the pre-existing 5 hmC and that produced by TET2 activity. In a second enzymatic step, following denaturation of the double-stranded DNA, the enzyme apolipoprotein B mRNA editing enzyme catalytic polypeptide-like 3A (APOBEC3A) is used to deaminate cytosines, but is unable to deaminate the oxidised or glycosylated forms of 5 mC and 5 hmC. Only unmethylated cytosines are deaminated to form uracil bases. Prior to the first enzymatic step, the DNA fragments may be generated from mechanical shearing and end-repaired, A-tailed, and ligated to sequencing adaptors, which can be carried out using the NEBNext® DNA Ultra II reagents (NEB), for example. Following the second enzymatic step, the deaminated single-stranded DNA may be amplified by PCR reactions, using polymerase such as NEBNext® Q5U™ which can amplify uracil containing templates, and the resulting library can be sequenced or analysed in an identical manner to the DNA sample generated by bisulfite sequencing. The output of EM-seq is generally the same as whole genome bisulfite sequencing, but with the use of less DNA-damaging reagents, which consequently reduces sample loss, and can outperform bisulfite-conversion prepared samples in coverage, sensitivity and accuracy of cytosine methylation calling. An illustrative EM-seq method is described by Vaisvila et al. (Genome Research; 2021; 31:1-10).

### Bisulfite Conversion-Based Methods

Bisulfite conversion utilizes the selective conversion of unmethylated cytosines to uracil when treated with sodium bisulfite. Denatured DNA is treated with sodium bisulfite, which converts all unmodified cytosines to uracil, and subsequent PCR amplification converts these residues to thymines. Analysing the produced DNA sequences can be done via many different methods, examples of which include but are not limited to: denaturing gel electrophoresis, single-strand conformation polymorphism, melting curves, fluorescent real-time PCR (MethyLight), MALDI mass spectrometry, array hybridization, and sequencing (e.g. Whole Genome Bisulfite Sequencing WGBS). Recently developed techniques such as SeqCap Epi enrich sequences of interest prior to sequencing that enables deeper coverage over a more focused area). Comparison of the abundance of sequences in a bisulfite-converted sample against those of an untreated control allows analysis of methylation at a target site, where the proportion of converted sequences is indicative of the level of methylation at the target site.

Further variants of the bisulfite conversion method are available that are able to distinguish 5 mC from the oxidised form 5-hydroxymethylcytosine (5 hmC), which behaves identically to 5 mC under standard bisulfite conversion, and to detect the further modification 5-formylcytosine (5 fC). These methods, such as oxBS-Seq and redBS-Seq, utilise oxidation and reduction of these markers to modify the susceptibility of each species to bisulfite conversion, and through comparative analysis quantify the amount of each modification at target loci.

### Selective Restriction Endonuclease Digestion Methods

Methods of analysing DNA methylation patterns exist may involve the use of restriction enzymes. These include, for example, restriction landmark genomic scanning (RLGS) (Costello et al., 2000; Nat Genet.; 24 (2): 132-8), methylation-sensitive representational difference analysis (MS-RDA) (Ushijima et al., Proc Natl Acad Sci USA. 1997 Mar. 18; 94 (6): 2284-9), and differential methylation hybridization (DMH) (Huang et al., Cancer Res. 1997 Mar. 15; 57 (6): 1030-4). Restriction endonucleases can be methylation dependent in their digestion activity. This specificity can be used to differentiate methylated and unmethylated sequences. Certain restriction enzymes, for example BstUI, HpaII and NotI are sensitive to methylated recognition sequences. Others, such as McrBC, are specific for methylated sequences.

As an example, differential methylation hybridisation (DMH) (Huang et al., as above]) requires an initial fragmentation of the genome with a bulk genome restriction enzyme, such as MseI, which fragments the genome into lengths of less than 200 bp. Following this step, the genome fragments are digested using a methylation-sensitive restriction endonuclease (MREs), or in some versions of the technique, a cocktail of MREs to improve coverage. Depending on the specificity of enzyme or enzymes used, either the methylated or the unmethylated sequences will be degraded. Digested sequences will not be amplified in a subsequent PCR step. The resultant PCR products are suitable for further processing and analysis by sequencing or microarray hybridisation in combination with fluorescent dyes.

Suitably, the present methods utilise a DNA methylation profile generating by a method comprising the use of one or more MREs.

Suitable comparators can be used to investigate methylation state between conditions. DNA from healthy subjects can be compared with aged or diseased subjects to detect changes in methylation state (Huang et al., Hum Mol Genet. 1999 March; 8 (3): 459-70). Alternatively, a methylation-insensitive version of the secondary digest enzyme, such as the HpaII isoschizomer MspI, can be used to generate a control sample, so that intra- or inter-genomic DNA methylation comparisons can be made (Khulan et al., Genome Res. 2006 August; 16 (8): 1046-55).

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. Alternatively, the method can encompass amplifying intact DNA after restriction enzyme digestion, thereby only amplifying DNA that was not cleaved by the restriction enzyme in the area amplified. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

Suitably, the digestion of nucleic acid is detected by selective hybridization of a probe or primer to the undigested nucleic acid. Alternatively, the probe selectively hybridizes to both digested and undigested nucleic acid but facilitates differentiation between both forms, e.g., by electrophoresis. Suitable detection methods for achieving selective hybridization to a hybridization probe include, for example, Southern or other nucleic acid hybridization.

Suitable hybridization conditions may be determined based on the melting temperature (Tm) of a nucleic acid duplex comprising the probe. The skilled artisan will be aware that optimum hybridization reaction conditions should be determined empirically for each probe, although some generalities can be applied. Preferably, hybridizations employing short oligonucleotide probes are performed at low to medium stringency. In the case of a GC rich probe or primer or a longer probe or primer a high stringency hybridization and/or wash is preferred. A high stringency is defined herein as being a hybridization and/or wash carried out in about 0.1×SSC buffer and/or about 0.1% (w/v) SDS, or lower salt concentration, and/or at a temperature of at least 65° C., or equivalent conditions. Reference herein to a particular level of stringency encompasses equivalent conditions using wash/hybridization solutions other than SSC known to those skilled in the art.

### Reduced Representation Bisulfite Sequencing (RRBS)

Reduced representation bisulfite sequencing (RRBS) enriches CpG-rich genomic regions using the MspI restriction enzyme-which cuts DNA at all CCGG sites, regardless of their DNA methylation status at the CG site-and enables the measurement of DNA methylation levels at 5% ˜ 10% of all CpG sites in the mammalian genome.

As such, the method involves digestion of DNA using the methylation-insensitive MspI prior the bisulfite conversion and sequencing. Using MspI to digest genomic DNA results in fragments that always start with a C (if the cytosine is methylated) or a T (if a cytosine was not methylated and was converted to a uracil in the bisulfite conversion reaction). This results in a non-random base pair composition. Additionally, the base composition is skewed due to the biased frequencies of C and T within the samples. Various software for alignment and analysis is available, such as Maq, BS Seeker, Bismark or BSMAP. Alignment to a reference genome allows the programs to identify base pairs within the genome that are methylated.

### Affinity Enrichment Based Methods

Distinction of methylated from unmethylated DNA can be accomplished by the use of antibodies, such as anti-5 mC, and/or methylated-CpG binding proteins, that contain a methyl-CpG-binding domain (MBD). The antibodies of MBD-domain proteins are able to specifically isolate methylated DNA over unmethylated DNA. Methods that utilize antibodies are commonly referred to as MeDIP, whilst methods utilizing methylated-CpG binding proteins are often known as MBD or MIRA approaches.

These methods require initial fragmentation of the genome, which can be carried out with bulk genome digest with an enzyme such as MseI, which cuts frequently, followed by affinity purification of methylated fragments. The input DNA can be compared to the purified methylated DNA by microarray hybridisation or sequencing to obtain comparative analysis of methylation levels at specific sites.

Further variants of affinity enrichment-based methods are available, such as MethylCap-Seq or MBD-Seq. These methods reduce sample complexity by using a salt gradient to elute methylated DNA fragments in a methy-CpG-abundance dependent manner, segregating CpG islands and other highly methylated loci from less CpG dense loci. The fractions can then be sequenced separately improving sequence coverage.

Single molecule sequencing-based and de novo methylation sequencing approaches

Contemporary sequencing methods are able to sequence single molecules directly. Single-molecule real-time (SMRT) DNA sequencing is available, for example the Sequel systems from Pacific Biosciences and has been shown to be able to identify modified bases such as methylated cytosine based on the polymerase kinetics. Nanopore sequencing devices, such as the MinION nanopore sequencer from Oxford Nanopore Technologies, which are able to individually sequence long strands of DNA, are also able to detect de novo base modifications, including methylation.

### DNA Methylation Sites

Suitably, a DNA methylation site may refer to the presence or absence of a 5 mC at a single cytosine, suitably a single CpG dinucleotide.

Suitably, a DNA methylation site may refer to the presence or absence of methylation (i.e. the number of 5 mC or percentage of 5 mC) across a plurality of CpG sites within a DNA region. Suitably, a DNA site methylation site may refer to the level of methylation (i.e. the number of 5 mC or percentage of 5 mC) across a plurality of CpG sites within a DNA region. A “DNA region” may refer to a specific section of genomic DNA. These DNA regions may be specified either by reference to a gene name or a set of chromosomal coordinates. Both the gene names and the chromosomal coordinates would be well known to, and understood by, the person of skill in the art.

Suitably, gene names and/or coordinates may be based on the “CanFam3.1” dog reference genome (https://www.ncbi.nlm.nih.gov/datasets/genome/GCF_000002285.3/, Lindblad-Toh et al.; Nature 438, 803-819 (2005)).

The DNA region may define a section of DNA in proximity to the promoter of a gene, for example. Promoter regions are known to be rich in CpG. By way of example, the DNA region may refer to about 3 kb upstream to about 3 kb downstream; about 2 kb upstream to about 2 kb downstream; about 2 kb upstream to about 1 kb downstream; about 2 kb upstream to about 0.5 kb downstream; about 1 kb upstream to about 0.5 kb downstream; about 0.5 kb upstream to about 0.5 kb downstream of a promoter. Suitably, the DNA region may refer to about 1 kb upstream to about 0.5 kb downstream of a promoter.

The DNA region may define other sections of DNA may be located-including, but not limited to, CpG islands, enhancers, open chromatin, transcription factor binding sites and miRNA promoter regions.

Suitably, the DNA region may comprise or consist of CpG sites that are less than about 5000, less than about 4000, less than about 3000, less than about 2000, less than about 1000, less than about 500, or less than about 200 bases apart.

Suitably, the DNA region may comprise or consist of CpG sites that are between about 200 to about 5000, about 200 to about 4000, about 200 to about 3000, about 200 to about 2000, or about 200 to about 1000 bases apart.

Suitably, the DNA region may comprise one or more CpG islands. Suitably, the DNA region may consist of a CpG island.

A “CpG island” may refer to a DNA region comprising at least 200 bp, a GC percentage greater than 50%, and an observed-to-expected CpG ratio greater than 60%.

Suitably, the DNA methylation sites do not comprise CpGs known to comprise a SNP at the CpG.

Reference to each of the genes/DNA regions detailed above should be understood as a reference to all forms of these molecules and to fragments or variants thereof. As would be appreciated by the person of skill in the art, some genes are known to exhibit allelic variation between individuals or single nucleotide polymorphisms. Variants include nucleic acid sequences from the same region sharing at least 90%, 95%, 98%, 99% sequence identity i.e. having one or more deletions, additions, substitutions, inverted sequences etc. relative to the DNA regions described herein. Accordingly, the present invention should be understood to extend to such variants which, in terms of the present applications, achieve the same outcome despite the fact that minor genetic variations between the actual nucleic acid sequences may exist between individuals. The present invention should therefore be understood to extend to all forms of DNA which arise from any other mutation, polymorphic or allelic variation.

In terms of screening for the methylation of these gene regions, it should be understood that the assays can be designed to screen for specific DNA. It is well within the skill of the person in the art to choose which strand to analyse and to target that strand based on the chromosomal coordinates. In some circumstances, assays may be established to screen both strands.

“Methylation status” may be understood as a reference to the presence, absence and/or quantity of methylation at a particular nucleotide, or nucleotides, within a DNA region. The methylation status of a particular DNA sequence (e.g. DNA region as described herein) can indicate the methylation state of every base in the sequence or can indicate the methylation state of a subset of the base pairs (e.g. of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.”

Suitably, DNA methylation may be determined using an EM-Seq strategy. In such methods, a methylation level can be determined as the fraction of ‘C’ bases out of ‘C′+′U’ total bases at a target CpG site “i” following an enzyme and APOBEC3A conversion treatment. In other embodiments, the methylation level can be determined as the fraction of ‘C’ bases out of ‘C′+′T’ total bases at site “i” following enzyme and APOBEC3A conversion treatment and subsequent nucleic acid amplification. The mean methylation level at each site may then be evaluated to determine if one or more threshold is met.

In some embodiments, in particular when bisulfite conversion and sequencing methods are used, a methylation level can be determined as the fraction of ‘C’ bases out of ‘C′+′U’ total bases at a target CpG site “i” following a bisulfite treatment. In other embodiments, the methylation level can be determined as the fraction of ‘C’ bases out of ‘C′+′T’ total bases at site “i” following a bisulfite treatment and subsequent nucleic acid amplification. The mean methylation level at each site may then be evaluated to determine if one or more threshold is met.

Alternatively, a methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of the methylation status. This is of particular use when it is desirable to compare the methylation status of a sequence in a sample to a threshold value.

The present invention is not to be limited by a precise number of methylated residues that are considered to indicative of biological age, because some variation between samples will occur. The present invention is also not necessarily limited by positioning of the methylated residue (e.g. a specific methylation site).

In one embodiment, a screening method can be employed which is specifically directed to assessing the methylation status of one or more specific cytosine residues or the corresponding cytosine at position n+1 on the opposite DNA strand.

### Enrichment and Detection Methods

Determining a DNA methylation profile may comprise a step of enriching a DNA sample for selected DNA regions. For example, the methods may comprise a step of enriching a DNA sample for DNA regions comprising the DNA methylation sites which comprise the DNA methylation profile.

Suitable enrichment methods are known in the art and include, for example, amplification or hybridisation based methods. Amplification enrichment typically refers to e.g. PCR based enrichment using primers against the DNA regions to be enriched. Any suitable amplification format may be used, such as, for example, polymerase chain reaction (PCR), rolling circle amplification (RCA), inverse polymerase chain reaction (iPCR), in situ PCR, strand displacement amplification, or cycling probe technology.

Hybridisation enrichment or capture-based enrichment typically refers to the use of hybridisation probes (or capture probes) that hybridise to DNA regions to be enriched.

The hybridisation probe(s) may be attached directly to a solid support, or may comprise a moiety, e.g. biotin, to allow binding to a solid support suitable for capturing biotin moieties (e.g. beads coated with streptavidin). In any case, DNA comprising sequence which is complementary to the probe may captured thus allowing to separate DNA comprising DNA regions of interest from not comprising the DNA regions of interest. Hence, such a capturing steps allows to enrich for the DNA regions of interest. For example, the DNA regions may be DNA regions in proximity to gene promoters.

An array used herein can vary depending on the probe composition and desired use of the array. For example, the nucleic acids (or CpG sites) detected in an array can be at least 10, 100, 1,000, 10,000, 0.1 million, 1 million, 10 million, 100 million or more. Alternatively or additionally, the nucleic acids (or CpG sites) detected can be selected to be no more than 100 million, 10 million, 1 million, 0.1 million, 10,000, 1,000, 100 or less. Similar ranges can be achieved using nucleic acid sequencing approaches such as those known in the art; e.g. Next Generation or massively parallel sequencing.

Suitably, an enrichment step may be performed before or after the step of separating or differentially treating methylated and unmethylated DNA.

As used herein, the term “enriching” or “enrichment” for “DNA” or “DNA regions” means a process by which the (absolute) amount and/or proportion of the DNA comprising the desired sequence(s) is increased compared to the amount and/or proportion of DNA comprising the desired sequence(s) in the starting material. In this regard, enrichment by amplification increases the amount and proportion of the desired sequence(s). Enrichment by capture-based enrichment increases the proportion of DNA comprising the desired sequence(s).

Following processing of the DNA to distinguish methylated and unmethylated sites, the present methods may further comprise the step of identifying the sites which were methylated or unmethylated (i.e. in the original sample).

The identification step may comprise any suitable method known in the art, for example array detection or sequencing (e.g. next generation sequencing).

A sequencing identification step preferably comprises next generation sequencing (massively parallel or high throughput sequencing). Next generation sequencing methods are well known in the art, and in principle, any method may be contemplated to be used in the invention. Next generation sequencing technologies may be performed according to the manufacturer's instructions (as e.g. provided by Roche, Illumina or Applied Biosystems).

In one embodiment, the sample is treated by converting DNA methylation using enzymatic reactions, performing whole genome library preparation and measuring the methylation profile by sequencing (EM-Seq).

In one embodiment, the sample is treated by converting DNA methylation using enzymatic reactions, performing whole genome library preparation, hybridizing the whole-genome-converted library preparation to capture probes (preferably capture probes capable of capturing DNA regions in proximity to gene promoters); and measuring the methylation profile by sequencing (EM-Seq).

Advantageously, the present methods may be performed using commercially available DNA methylation arrays. In one embodiment, the sample is treated by converting DNA methylation using bisulfite conversion, optionally amplifying the converted DNA, before labelling (e.g. with fluorescent dye) and hybridizing to a methylation array (e.g. mammalian methylation array). Suitable methylation arrays are available from e.g. Illumina and are described in WO20150705 and Arneson et al. (Nature Communications; 13 (782); 2022).

### DNA Methylation Profile

A “DNA methylation profile” or “methylation profile” may refer to the presence, absence, quantity or level of 5 mC at one or more DNA methylation sites. Preferably, “methylation profile” refers to the presence, absence, quantity or level of 5 mC at a plurality of DNA methylation sites. Thus, the presence, absence, quantity or level of 5 mC at each individual DNA methylation site within the plurality of sites may be assessed and contribute to the determination of the mortality risk and/or probability of a healthy lifespan of the dog. The quality and/or the power of the methods may thus be improved by combining values from multiple DNA methylation markers.

Suitably, the present biological clock comprises the methylation profile from a plurality of methylation sites.

Suitably, presence or absence of 5 mC from at least 3, at least 5, at least 10, at least 20, at least 50, at least 100, at least 200, at least 500, at least 1000, at least 2000, at least 5000, at least 10000, at least 50000, at least 10000, at least 250000, or at least 500000 DNA methylation sites may be used to determine mortality risk and/or probability of a healthy lifespan (i.e. biological age) of the dog.

Suitably, the methylation profile may refer to the presence or absence of 5 mC from at least 100, at least 200, at least 500, at least 1000 or at least 2000 DNA methylation sites.

Suitably, the methylation profile may refer to the presence or absence of 5 mC from about 100, about 200, about 500, about 1000 or about 2000 DNA methylation sites.

In order to generate a biological clock for determining mortality risk and/or probability of a healthy lifespan, an initial methylation profile may be processed or streamlined to produce a restricted methylation profile which is then used to generate the biological clock.

By way of example, an initial methylation profile may be processed or streamlined by—for example—using DNA regions rather than individual cytosines, by selecting a subset of methylation sites that are associated with a particular physiological or biochemical pathway, performing a correlation analysis and retaining one or more representative DNA methylation sites per cluster, or performing differential analysis to pre-select DNA methylation sites or retain DNA methylation sites that vary more between young and old dogs,

For example, the DNA region(s) may be any DNA region(s) as defined herein.

Suitably, the methylation profile may refer to DNA methylation sites of genes that are associated with a particular physiological or biochemical pathway. As such, the methylation profile may enable a biological age of a particular tissue, organ, or physiological system to be determined. Determining a biological age for a particular tissue, organ or physiological system may advantageously allow the method to be utilised in a way which focuses on pathologies and diseases of that tissue, organ or physiological system. For example, if a particular breed of dog is known to be associated with muscular or cardiovascular disease, it may be advantageous to determine a biological age for that physiological system.

Suitably, the physiological system may be the inflammatory, muscular, cardiovascular, and/or neurological system.

A biological age for a particular tissue, organ, or physiological system may be determined using a DNA methylation profile comprising, or consisting of, methylation sites from genes that are preferentially or specifically expressed by that tissue, organ, or physiological system. Classifications of genes by a particular tissue, organ, or physiological system are publicly available at, for example, Gene Ontology (http://geneontology.org/), the KEGG pathway database (https://www.genome.jp/kegg/), or MSIgDB (https://www.gsea-msigdb.org/gsea/msigdb/index.jsp).

In some embodiments, a threshold selects those sites having the highest-ranked mean methylation values for epigenetic age predictors. For example, the threshold can be those sites having a mean methylation level that is the top 50%, the top 40%, the top 30%, the top 20%, the top 10%, the top 5%, the top 4%, the top 3%, the top 2%, or the top 1% of mean methylation levels across all sites “i” tested for a predictor, e.g., a biological clock.

Alternatively, the threshold can be those sites having a mean methylation level that is at a percentile rank greater than or equivalent to 50, 60, 70, 80, 90, 95, 96, 97, 98, or 99. In other embodiments, a threshold can be based on the absolute value of the mean methylation level. For instance, the threshold can be those sites having a mean methylation level that is greater than 99%, greater than 98%, greater than 97%, greater than 96%, greater than 95%, greater than 90%, greater than 80%, greater than 70%, greater than 60%, greater than 50%, greater than 40%, greater than 30%, greater than 20%, greater than 10%, greater than 9%, greater than 8%, greater than 7%, greater than 6%, greater than 5%, greater than 4%, greater than 3%, or greater than 2%. The relative and absolute thresholds can be applied to the mean methylation level at each site “i” individually or in combination. As an illustration of a combined threshold application, one may select a subset of sites that are in the top 3% of all sites tested by mean methylation level and also have an absolute mean methylation level of greater than 6%. The result of this selection process is a DNA methylation profile, of specific hypermethylated sites (e.g., CpG sites) that are considered the most informative for mortality risk and/or probability of a healthy lifespan determination.

Suitably, the DNA methylation profile used to determine a mortality risk and/or probability of a healthy lifespan according to the present invention may comprise at least one methylation site as listed in Table 3.

Suitably, the methylation site(s) may be defined as the methylation marker present in any one or more of SEQ ID NO: 1-149. SEQ ID NO: 1-149 show the sequence adjacent to the methylation marker in the “CanFam3.1” dog reference genome (https://www.ncbi.nlm.nih.gov/datasets/genome/GCF000002285.3/, Lindblad-Toh et al.; Nature 438, 803-819 (2005)) with the “CG” methylation marker positioned at the terminus of the sequence (at the start or the end of the sequence depending on whether the site is on the plus or minus strand in the reference genome). The position of the “CG” methylation marker is provided in Table 3. In addition, the respective CGid is also provided for each “CG” methylation marker (see Arneson et al.; Nature Communications; 13 (783); 2022 and https://github.com/shorvath/MammalianMethylationConsortium/tree/v1.0.0).

Suitably, the methylation sites may be defined by the CGstart and CGend columns in Table 3. For example, for DNA methylation site number 1 (SEQ ID NO: 1), the sequence provided is chr9: 22470282-22470331, and the methylation marker is chr9: 22470282-22470283.

Suitably, the DNA methylation profile may comprise at least 3, at least 5, at least 10, at least 20, at least 50, at least 100, at least 125, or preferably each of the methylation sites as listed in Table 3.

Suitably, the DNA methylation profile comprises at least one methylation site selected from the sites numbered 1-124 as listed in Table 3.

Suitably, the DNA methylation profile comprises at least 3, at least 5, at least 10, at least 20, at least 50, at least 100, at least 125, or each of the methylation sites as listed in Table 3.

Suitably, the DNA methylation profile comprises at least 3, at least 5, at least 10, at least 20, at least 50, at least 100, or each of the methylation sites from the sites numbered 1-124 as listed in Table 3.

Suitably, the DNA methylation profile may comprise the methylation sites as listed in Table 4. Suitably, the DNA methylation profile may comprise methylation sites 1 to 3 as listed in Table 4.

Suitably, the DNA methylation profile may comprise the methylation sites as listed in Table 5. Suitably, the DNA methylation profile may comprise methylation sites 1 to 3 and 4 to 7 as listed in Table 5.

Suitably, the DNA methylation profile may comprise the methylation sites as listed in Table 6. Suitably, the DNA methylation profile may comprise methylation sites 1 to 3, 4 to 7 and 8 to 22 as listed in Table 6.

Suitably, the DNA methylation profile may comprise the methylation sites as listed in Table 7. Suitably, the DNA methylation profile may comprise methylation sites 1 to 3, 4 to 7, 8 to 22 and 23 to 38 as listed in Table 7.

Determination of DNA methylation sites/DNA methylation profiles indicative of mortality risk and/or probability of a healthy lifespan

The present invention comprises utilising a DNA methylation profile to determine a mortality risk and/or probability of a healthy lifespan of a dog wherein the DNA methylation profile comprises at least one methylation site as listed in Table 3. As such, the present invention comprises utilising a DNA methylation profile to generate a biological clock which is associated with mortality risk and/or probability of a healthy lifespan. The present biological clock may also be referred to as an ‘epigenetic clock’.

The provision of DNA methylation sites or a DNA methylation profile that is indicative of mortality risk and/or probability of a healthy lifespan may be achieved through training datasets and machine learning approaches, for example. Suitably, the machine learning approaches may be supervised machine learning approaches.

By way of example, DNA methylation sites or a DNA methylation profile may be trained against a dataset comprising dogs of a known mortality outcome (alive or dead) and chronological age. Suitably, the DNA methylation sites or a DNA methylation profile may be trained against a dataset comprising dogs of a known mortality outcome and chronological age in combination with known breed and/or sex.

For example, models for DNA methylation sites or a DNA methylation profile indicative of mortality risk and/or probability of a healthy lifespan may be provided by training a dataset of methylation status at a plurality of DNA methylation sites against a training dataset of dogs with a known mortality outcome (alive or dead) and chronological age using a machine learning framework, and testing against a with—held cohort to validate the veracity of the model.

The machine learning framework may comprise fitting a penalised model to a training dataset of dogs with a known mortality outcome (alive or dead) and chronological age (and optionally breed and/or sex); for example using glmnet R package.

The machine learning framework may comprise fitting a penalised model to a training dataset of dogs with a known mortality outcome (alive or dead, age at death) and chronological age (and optionally breed and/or sex); for example using glmnet R package.

Suitably, the penalised model may be a penalized Cox regression, a Least Angle Regression path of solution (LARS) Cox regression or a penalized survival model; for example.

The machine learning framework may comprise fitting a penalized Cox regression to a training dataset of dogs with a known mortality outcome (alive or dead) and chronological age (and optionally breed and/or sex); for example using glmnet R package.

Suitably, the machine learning framework may comprise fitting a penalised model, preferably a penalized Cox regression, of known mortality outcome (alive or dead)/survival explained by a DNA methylation profile and chronological age, (and optionally breed and/or sex).

Suitably, the machine learning framework may comprise fitting a penalised model, preferably a penalized Cox regression, of known mortality outcome (alive or dead)/survival explained by a DNA methylation profile, chronological age, breed and sex.

As used herein ‘known mortality outcome (alive or dead)’ may also be referred to as ‘survival’.

Suitably, the machine learning framework may be used to determine a model comprising a set of DNA methylation sites or a DNA methylation profile that is indicative of mortality risk and/or probability of a healthy lifespan.

Suitably, the machine learning framework may generate a predicted hazard (e.g. a predicted hazard ratio); for example as generated by a penalized Cox regression. This can be converted to a biological/epigenetic age using methods which are known in the art; for example by fitting a linear model to explain chronological age by the predicted hazards.

The model may comprise the methylation status at a plurality of DNA methylation sites; wherein the methylation status at each site is considered in the model by multiplying by a coefficient value.

Suitably, sex is may be coded as a numerical value with 0 for female and 1 for male.

Suitably, breed may be coded as a numerical value with 0 for small breeds and 1 for medium breeds.

The biological age of the dog may be expressed in terms of years, months, days, etc.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values. Such methods include, for example, computation of two gompertz or weibull functions on a training set (e.g. where the status of the dog (alive or dead) is known), one that models survival as a function of the methylation profile, chronological age, breed class (small or medium dog) and sex (model 1) and a second function that only considers chronological age, breed class and sex (model 2). These models may be fit using the flexsurv package (v 2.1) in the R software environment.

The biological age may be defined as the time variable (“chronological age”) at which the survival probability of the animal given by model 2 is equal to the survival probability at their chronological age given by the model 1.

Models for DNA methylation sites or a DNA methylation profile indicative of mortality risk and/or probability of a healthy lifespan may be provided by training a dataset of methylation status at a plurality of DNA methylation sites against a PhenoAge predicted at the age of DNA sample collection, and testing against a withheld cohort to validate the veracity of the model.

Methods for determining the PhenoAge of a dog or cat are described in PCT/EP2023/061058 and PCT/EP2023/061059; respectively. Calculation of PhenoAge takes into account the direct predictive value of blood biomarkers on mortality risk and/or probability of a healthy lifespan. By way of example, a given biomarker may not directly correlate with chronological age, but may be indicative of a particular pathological condition and thus an increased mortality risk and/or a probability of a reduced healthy lifespan.

Determining the PhenoAge of a dog may comprise determining the level of one or more biomarker(s) in one or more samples obtained from the dog, wherein the one or more biomarker(s) is selected from white blood cell count, serum albumin, serum alkaline phosphatase, serum creatine kinase, haemoglobin, haematocrit, mean corpuscular haemoglobin, serum glucose, mean red cell volume, serum globulin, serum calcium, platelet count, and/or red blood cell count.

Suitably, the PhenoAge of a dog may be provided by
a. determining the level of the following biomarkers; white blood cell count, serum albumin, serum alkaline phosphatase, serum creatine kinase, haemoglobin, haematocrit, mean corpuscular haemoglobin, serum glucose, mean red cell volume, and serum globulin in one or more samples obtained from the dog; and
b. determining a phenotypic age (Phenoage) of the dog using formula (1):

$$Phenoage = ln ( \frac{{γ}_{breed} * {e}^{xb} * \{ {e}^{γ * age} - 1 \}}{{e}^{\{ breed * {β}_{breed 2} \} + \{ sex * {β}_{sex 2} \} + {β}_{02}} * γ} + 1 ) * \frac{1}{{γ}_{breed}}$$

where xb is the sum of the value of each biomarker(s), sex and breed multiplied by their respective coefficients according to formula (2):

$$xb = {\sum }_{u = 1}^{p} {x}_{u} {β}_{u} + {β}_{0}$$

wherein sex is coded as a numerical value with 0 for female and 1 for male, wherein breed is coded as a numerical value with 0 for small breeds and 1 for medium breeds, and wherein the phenotypic age is used to determine a mortality risk and/or probability of a healthy lifespan for the dog.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values for use in above formula. Such methods include, for example, computation of two gompertz or weibull functions on a training set (e.g. where the status of the dog (alive or dead) is known), one that models survival as a function of the selected biomarkers, chronological age, breed class (small or medium dog) and sex (model 1) and a second function that only considers chronological age, breed class and sex (model 2). These models may be fit using the flexsurv package (v 2.1) in the R software environment.

Suitably, a negative coefficient for a given biomarker means that a higher level of the biomarker has a positive effect on reducing mortality risk and/or a lower level of the biomarker has a negative effect on reducing mortality risk. Suitably, a positive coefficient for a given biomarker means that a higher level of the biomarker has a negative effect on reducing mortality risk and/or a lower level of the biomarker has a positive effect on reducing mortality risk.

Illustrative coefficients and γ and γ_{breed} values are provided in the following table.

| | Coefficient |
|---|---|
| γ | 0.491790219 |
| β₀ | −6.036261473 |
| β White blood cells count | 0.091862564 |
| β Hemoglobin | −0.009131623 |
| β Mean Red Cell Volume | −0.007486146 |
| β Hematocrit | −0.018418391 |
| β Mean Corpuscular Hemoglobin | −0.128195615 |
| β Serum Glucose | 0.009169677 |
| β Serum Globulin | 0.132755858 |
| β Serum Creatine Kinase | 0.332818902 |
| β Serum Albumin | −0.744060565 |
| β Serum Alkaline Phosphatase | 0.262594338 |
| β breed | 1.138018960 |
| β Sex | 0.151826455 |
| γ_{breed} | 0.5668399 |
| β₀₂ | −9.5204440 |
| β_{breed2} | 1.2299804 |
| βₛₑₓ₂ | 0.2678798 |

The phenotypic age may be defined as the time variable (“chronological age”) at which the survival probability of the animal given by model 2 is equal to the survival probability at their chronological age given by the model 1.

The phenotypic age (i.e. phenoage) of the dog may be expressed in terms of years, months, days, etc.

The biomarkers used to determine PhenoAge can be determined using standard methods in the art and are typically measured as part of standard blood tests to determine the disease status of an animal. For example, the biomarkers are commonly determined as part of a standard clinical complete blood count (cbc) and standard clinical blood chemistry analysis.

Suitably, a model for DNA methylation sites or a DNA methylation profile indicative of mortality risk and/or probability of a healthy lifespan trained against a PhenoAge may be provided in a two-step process.

In a first step, a machine learning framework may comprise fitting a penalised model of a phenotypic age (PhenoAge) explained by one or more blood biomarkers as described herein and chronological age (and optionally sex and/or breed); for example using glmnet R package. Preferably, the machine learning framework may comprise fitting a penalised model of a phenotypic age (PhenoAge) explained by one or more blood biomarkers as described herein, chronological age, sex and breed.

Suitably, the penalised model may be a penalized Cox regression, a Least Angle Regression path of solution (LARS) Cox regression or a penalized survival model; for example.

The machine learning framework may comprise fitting a penalised Cox regression of a phenotypic age (PhenoAge) explained by one or more blood biomarkers as described herein, chronological age, sex and breed.

In a second step, the machine learning framework may comprise fitting a penalised regression of PhenoAge explained by a DNA methylation. Suitably, the machine learning framework may comprise fitting a penalised regression of PhenoAge explained by a DNA methylation profile.

The penalised regression may be an elastic net regression.

The term “one or more biomarkers” as used herein may include at least one, at least two, at least three, at least four, at least five, at least six, at least seven, at least eight, at least nine, at least ten, at least eleven, at least twelve or at least thirteen biomarkers.

The term “one or more biomarkers” as used herein may include one, two, three, four, five, six, seven, eight, nine, ten, eleven, twelve or thirteen biomarkers.

Suitably, DNA methylation sites or a DNA methylation profile may be combined with the level of one or more blood biomarkers described herein in order to generate a model indicative of mortality risk and/or probability of a healthy lifespan. For example, a model comprising a combination of a DNA methylation profile and the level of one or more blood biomarkers described herein may be provided by training a dataset of methylation status at a plurality of DNA methylation sites and the level of one or more blood biomarkers against a training dataset of dogs with a known mortality outcome (alive or dead) and chronological age, and testing against a with-held cohort to validate the veracity of the model.

The machine learning framework may comprise fitting a penalised regression to a training dataset of dogs with a known mortality outcome (alive or dead) and chronological age (and optionally breed and/or sex); for example using glmnet R package.

The machine learning framework may comprise fitting a penalized Cox regression to a training dataset of dogs with a known mortality outcome (alive or dead) and chronological age (and optionally breed and/or sex); for example using glmnet R package.

Suitably, the machine learning platform may comprise one or more deep neural networks. Neural Networks are collections of neurons (also called units) connected in an acyclic graph. Neural Network models are often organized into distinct layers of neurons. For most neural networks, the most common layer type is the fully-connected layer in which neurons between two adjacent layers are fully pairwise connected, but neurons within a single layer share no connections. One of the main features of deep neural networks is that neurons are controlled by non-linear activation functions. This non-linearity combined with the deep architecture make possible more complex combinations of the input features leading ultimately to a wider understanding of the relationships between them and as a result to a more reliable final output. Deep neural networks have been applied for many types of data ranging from structural data to chemical descriptors or transcriptomics data.

Suitably, the machine learning platform comprises one or generative adversarial networks. Suitably, the machine learning platform comprises an adversarial autoencoder architecture. Suitably, the machine learning platform comprises a feature importance analysis for ranking DNA methylation site by their importance in biological age determination.

The biological age of the dog may be expressed in terms of years, months, days, etc.

Preferably, the mortality risk and/or probability of a healthy lifespan is represented as the difference between biological age and chronological age of the dog.

### Comparison to a Reference or Control

The present method may further comprise a step of comparing the difference in DNA methylation at one or more sites in the test sample to one or more reference or controls. The presence or absence of DNA methylation at one or more sites in the reference or control may be associated with a pre-defined mortality risk and/or probability of a healthy lifespan (i.e. biological age). In some embodiments, the reference value is a value obtained previously for a subject or group of subjects with a known mortality risk and/or probability of a healthy lifespan (i.e. biological age). The reference value may be based on a known DNA methylation status at one or more sites, e.g. a mean or median level, from a group of subjects with known mortality status (alive or dead), chronological age, breed, and/or sex.

Combining the DNA Methylation Profile with Further Measures and/or Characteristics

Suitably, the present method further comprises combining the DNA methylation profile with one or more of the chronological age, breed and/or sex of the dog. By combining this information, a biological age may be determined which is associated with mortality risk and/or probability of a healthy lifespan.

### Subject Stratification

The biological age determined by the method of the present invention may also be compared to one or more pre-determined thresholds (i.e. difference to chronological age). Using such thresholds, subjects may be stratified into categories which are indicative of determined risk, e.g. low, medium or high determined risk. The extent of the divergence from the thresholds is useful to determine which subjects would benefit most from certain interventions. In this way, dietary intervention and modification of lifestyle can be optimised.

### Method for Selecting/Monitoring a Lifestyle Regime, Dietary Regime or Therapeutic Intervention of a Subject

In a further aspect, the present invention provides a method for selecting a lifestyle regime, dietary regime or therapeutic intervention for a subject. The modification in lifestyle may be any change as described herein, e.g. a dietary intervention and/or a change in exercise regime. The modification in lifestyle may be administration of a therapeutic modality.

The lifestyle regime, dietary regime or therapeutic intervention may be applied to the dog for any suitable period of time. After said period of time, the dog's mortality risk and/or probability of a healthy lifespan may be determined again using the present method in order to determine the efficacy of the lifestyle regime, dietary regime or therapeutic intervention for reducing the mortality risk and/or increasing probability of a healthy lifespan of the dog. By way of example, the lifestyle regime, dietary regime or therapeutic intervention may be applied for at least 2, at least 4, at least 8, at least 16, at least 32, or at least 64 weeks. The lifestyle regime, dietary regime or therapeutic intervention may be applied for at least 3, at least 6, at least 12, at least 24, at least 36, at least 48 or at least 60 months.

The lifestyle regime, dietary regime or therapeutic intervention may be referred to as an anti-aging lifestyle regime, dietary regime or therapeutic intervention.

Preferably the modification is a dietary intervention as described herein. By the term “dietary intervention” it is meant an external factor applied to a subject which causes a change in the subject's diet. More preferably the dietary intervention includes the administration of at least dietary product or dietary regimen or a nutritional supplement.

The dietary intervention may be a meal, a regime of meals, a supplement or a regime of supplements or combinations of a meal and a supplement, or combinations of a meal and multiple supplements.

The dietary intervention or dietary product described herein may be any suitable dietary regime, for example, a calorie-restricted diet, a senior diet, a low protein diet, a phosphorous diet, low protein diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a senior diet, or a low protein diet. Suitably, the dietary intervention or dietary product may be a calorie-restricted diet. Suitably, the dietary intervention or dietary product may be a low protein diet.

A dietary intervention may be determined based on the baseline maintenance energy requirement (MER) of the dog. Suitably, the MER may be the amount of food that stabilizes the dog's body weight (less than 5% change over three weeks).

By way of example, it is generally understood that younger, growing dogs benefit from a high energy/high protein diet; however, older dogs may have a lower energy requirement and therefore diets can be appropriately modified. In particular, many manufacturers produce a ‘senior’ range of dog food which is lower in calories, higher in fibre but has suitable levels of protein and fat for an older dog.

Suitably, a calorie-restricted diet may comprise about 50%, about 55%, about 60%, about 65%, about 75%, about 80%, about 85%, or about 90% of the dog's MER. Suitably, a calorie-restricted diet may comprise about 60% or about 75% of the dog's MER.

Suitably, a low-protein diet may comprise less than 20% protein (% dry matter). For example, a low-protein diet may comprise less than 19% protein (% dry matter).

These diets are generally recommended based upon the chronological age of a dog. For example, it may be recommended that a dog is switched to a senior diet around 7 or 8 years old. However, in the context of the present invention, the determination of an increased mortality risk for a dog compared to what would be expected given its chronological age may allow a determination to switch the dog to a senior diet at an earlier age. In contrast, a dog with a reduced mortality risk compared to its chronological age may be able to stay on an adult diet for longer.

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

Modifying a lifestyle of the subject also includes indicating a need for the subject to change lifestyle, e.g. prescribing more exercise. Similar to a dietary intervention, the determination of an increased mortality risk for a dog compared to what would be expected given its chronological age may allow a determination a switch the dog to an appropriate exercise regime.

Modifying a lifestyle of the subject also includes selecting or recommending a therapeutic modality or regimen. The therapeutic modality or regimen may be a modality useful in treating and/or preventing—for example—arthritis, dental diseases, endocrine disorders, heart disease, diabetes, liver disease, kidney disease, prostate disorders, cancer and behavioural or cognitive disorders. Suitably, prophylactic therapies may be administered to a dog identified as being at risk of such disorders due to increased mortality risk and/or on the basis of particular biomarkers which are known to be associated with disease-relevant pathways. In other embodiments, dogs determined to be at risk of certain conditions (due to increased mortality risk) and/or on the basis of particular biomarkers which are known to be associated with disease-relevant pathways) may be monitored more regularly so that diagnosis and treatment can begin as early as possible.

The present invention is also directed to monitoring and/or determining the efficacy of an anti-ageing therapy or developing an anti-ageing therapy. The anti-aging therapy may comprise, for example, a “rejuvenation” intervention. A rejuvenation intervention aims to cause a reduction in the epigenetic or biological age of the subject. Suitably, the rejuvenation intervention may reprogram epigenetic age to that of a very young dog. Examples of such rejuvenation interventions include, but are not limited to, a gene therapy that reprograms epigenetic age, suitably to that of a very young dog. The present methods to monitor and/or determine the efficacy of a lifestyle regime, dietary regime or therapeutic intervention or develop a lifestyle regime, dietary regime or therapeutic intervention to reduce biological age are particularly applicable to this aspect.

The present invention may thus advantageously enable the identification of dogs that are expected to respond particularly well to a given intervention (e.g. lifestyle regime, dietary regime or therapeutic intervention). The intervention can thus be applied in a more targeted manner to dogs that are expected to respond.

In one aspect, the present invention provides a method for determining the efficacy of a lifestyle regime, dietary regime or therapeutic intervention for reducing the mortality risk and/or increasing the probability of a healthy lifespan of a dog, said method comprising: a) applying a lifestyle regime, dietary regime or therapeutic intervention to the dog, wherein the lifestyle regime, dietary regime or therapeutic intervention has been selecting according to the method of the invention; b) after a time period of applying the lifestyle regime, dietary regime or therapeutic intervention to the dog; determining a mortality risk and/or probability of a healthy lifespan of the dog using a DNA methylation profile from a sample obtained from the dog wherein the DNA methylation profile comprises at least one methylation site as listed in Table 3; c) determining if there has been a change in the mortality risk of the dog after the time period of following the lifestyle regime, dietary regime or therapeutic intervention.

In a further aspect the invention provides a method for determining the efficacy of a lifestyle regime, dietary regime or therapeutic intervention for reducing the mortality risk and/or increasing the probability of a healthy lifespan of a dog, said method comprising: a) determining a mortality risk and/or probability of a healthy lifespan for the dog using a DNA methylation profile from a sample obtained from the dog wherein the DNA methylation profile comprises at least one methylation site as listed in Table 3; b) applying a lifestyle regime, dietary regime or therapeutic intervention selected based on the mortality risk and/or probability of a healthy lifespan determined in step a) to the dog; c) after a time period of applying a lifestyle regime, dietary regime or therapeutic intervention to the dog; determining a mortality risk and/or probability of a healthy lifespan of the dog using a DNA methylation profile from a sample obtained from the dog wherein the DNA methylation profile comprises at least one methylation site as listed in Table 3; d) determining if there has been a change in the mortality risk and/or probability of a healthy lifespan of the dog between step a) and step c).

Suitably, the lifestyle regime, dietary regime or therapeutic intervention may have been applied to the dog for a period before the first mortality risk and/or probability of a healthy lifespan is determined; however, the effectiveness of the lifestyle regime, dietary regime or therapeutic intervention for improving the mortality risk and/or probability of a healthy lifespan of the dog (i.e. reducing the mortality risk and/or increasing the probability of a healthy lifespan) may still be monitored by determining a mortality risk and/or probability of a healthy lifespan at two or more times during the application of the lifestyle regime, dietary regime or therapeutic intervention.

Suitably, the present methods may comprise an ‘ecosystem’; in particular a digital ecosystem. Suitably, the present methods may comprise providing a sample obtained from the dog, optionally using a kit according to present invention; and (b) providing the sample (e.g. by mailing) for subsequent DNA extraction for the measurement of DNA methylation in the extracted DNA from the sample to obtain a DNA methylation profile.

The DNA methylation profile may then be used according to any of the present methods; preferably using a computer system or a computer program product according to the present invention.

The computer system or computer program may then prepare and share a report detailing the outcome of analysis/method in the form of e.g. selecting or recommending a suitable lifestyle regime, dietary regime or therapeutic intervention for a dog or any other outcome of the present methods.

Suitably, the sample may be a sample that can be obtained at home by a dog owner (e.g. not requiring a veterinarian or health-care professionals). Suitably, the sample may be a hair follicle, buccal swab or saliva sample.

### Use of a Dietary Intervention

In one aspect, the present invention provides a dietary intervention for use in reducing the mortality risk and/or increasing the probability of a healthy lifespan of a dog, wherein the dietary intervention is administered to a dog with a mortality risk and/or probability of a healthy lifespan determined by the present method.

In another aspect, the present invention provides the use of a dietary intervention to reduce the mortality risk and/or increase the probability of a healthy lifespan of a dog, wherein the dietary intervention is administered to a dog with a mortality risk and/or probability of a healthy lifespan determined by the present method.

As described herein, the dietary intervention may be a dietary product or dietary regimen or a nutritional supplement.

### Computer Program Product

The present methods may be performed using a computer. Accordingly, the present methods may be performed in silico.

Suitably, the computer may prepare and share a report detailing the outcome of the present methods.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the mortality risk and/or probability of a healthy lifespan of a dog as described herein.

In one embodiment, the user inputs into the device levels of one or more of DNA methylation markers as defined herein, optionally along with chronological age, breed and sex. The device then processes this information and provides a determination of a biological age for the dog. Alternatively, the device then processes this information and provides a determination of a suitable lifestyle regime, dietary regime or therapeutic intervention for the dog based on the biological age.

The device may generally be a server on a network. However, any device may be used as long as it can process biomarker data and/or additional parameters or characteristic data using a processor, a central processing unit (CPU) or the like. The device may, for example, be a smartphone, a tablet terminal or a personal computer and output information indicating the determined biological age for the dog or a determination of a suitable lifestyle regime, dietary regime or therapeutic intervention for the dog based on the biological age.

Those skilled in the art will understand that they can freely combine all features of the present invention described herein, without departing from the scope of the invention as disclosed.

### Examples

The invention will now be further described by way of examples, which are meant to serve to assist the skilled person in carrying out the invention and are not intended in any way to limit the scope of the invention.

### Example 1-Identification of DNA Methylation Sites

Whole blood samples from a canine cohort comprising data from blood and buccal swab samples were analysed by performing DNA extraction, converting DNA methylation by using bisulfite conversion, amplifying the converted DNA. Then DNA was hybridized to mammalian methylation arrays (Illumina) and labelled with fluorescent dye. After the hybridization step, the array was washed and scanned using a microarray scanner iScan. Raw data were read and normalized using sesame R package (Zhou W, Triche TJ, Laird PW, Shen H (2018). “SeSAMe: reducing artifactual detection of DNA methylation by Infinium BeadChips in genomic deletions.” Nucleic Acids Research, gky691. doi: 10.1093/nar/gky691.)

Raw data were read and normalized using sesame R package (Zhou W, Triche TJ, Laird PW, Shen H (2018). “SeSAMe: reducing artifactual detection of DNA methylation by Infinium BeadChips in genomic deletions.” Nucleic Acids Research, gky691. doi: 10.1093/nar/gky691.)

Several steps were then taken to process the array data:
Outliers in the inter array correlation were removed
Samples with incorrect Predicted Species were excluded from the dataset.
Misclassified samples and technical replicates were also eliminated to maintain data accuracy.

Beta value preparation:

To reduce the dimensionality of the beta value matrix, a filtering approach was applied based on the reliability of probes across technical replicates. This involved training 13 pairs of technical replicates and performing a regression analysis using the model beta ˜ ReplicateID. Through this process, probes that exhibited greater variation in methylation levels between biological replicates compared to technical replicates were removed. Package limma was used for this analysis.

Probes that had a detection p-value larger than 0.05 in 10% of the samples were also removed. This filtering process aimed to eliminate less reliable probes.

Finally, all cpg ID that did not match the dog genome were removed.

A total of 12009 probes are selected by this process.

### Example 2-Determination of Blood Biomarkers Associated with Mortality Risk in Dogs

Predictive blood biomarkers were determined from a biomarker panel consisting of a standard clinical complete blood count (cbc) and standard clinical blood chemistry analysis. Serum samples were taken after overnight fasting and measured using standard veterinary clinical practice.

TABLE 1
Clinical complete blood count (cbc)
and clinical blood chemistry analysis

| Parameter name | Unit of measure |
|---|---|
| Hematocrit | % |
| Hemoglobin | g/dL |
| Mean Corpuscular Hemoglobin | Pg |
| Mean Corpuscular Hemoglobin concentration | g/dL |
| Mean Red Cell Volume | fL |
| Platelet | 10\hat{ }3/uL |
| Red blood cells | 10\hat{ }3/uL |
| White blood cells | 10\hat{ }3/uL |
| Serum Albumin Plus | g/dL |
| Serum Alkaline Phosphatase * | U/L |
| Serum ALT * | U/L |
| Serum AST * | U/L |
| Serum Calcium | mg/dL |
| Serum Chloride | mmol/L |
| Serum Cholesterol | mg/dL |
| Serum Cretaine Kinase * | IU/L |
| Serum Creatinine, Jaffe Method * | mg/dL |
| Serum GGT * | g/dL |
| Serum Globulin | g/dL |
| Serum Glucose | mg/dL |
| Serum Magnesium | mg/dL |
| Serum Phosphorus | mg/dL |
| Serum Potassium | mmol/L |
| Serum Sodium | mmol/L |
| Serum Total Bilirubin * | mg/dL |
| Serum Total Protein | g/dL |
| Serum Triglycerides * | mg/dL |
| Serum Urea Nitrogen * | mg/dL |

* value were log-transformed using natural logarithm.

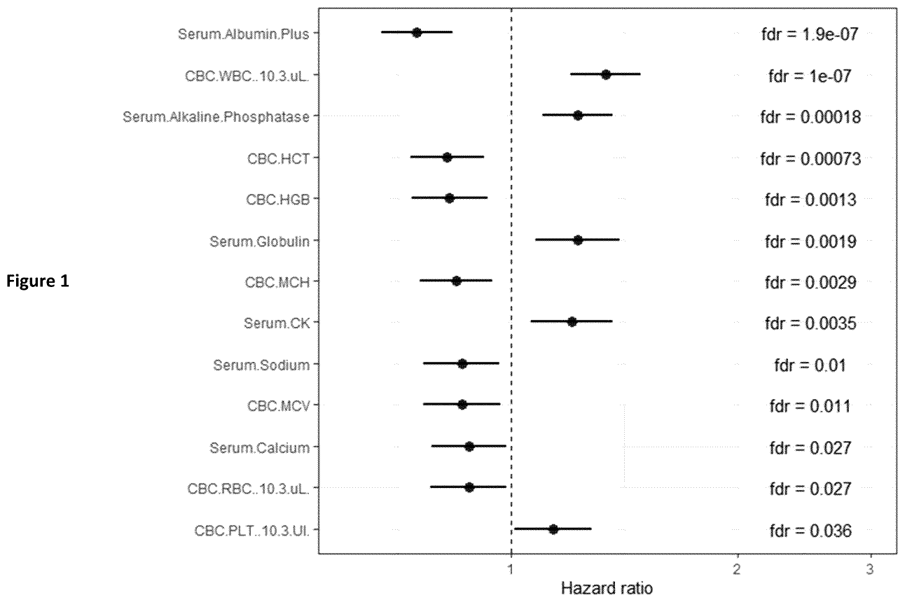

We used a longitudinal study of dogs for which we have repeated measurement of these parameters as well as information about the status of the dog (alive or dead), their sex and their breed. We first categorized breeds as small or medium based on the average weight of adult dogs of this breed (below 10 kg or above 10 kg, respectively). Then we organized the data using the R programming language. For each dog, we recorded the biomarkers as time dependent covariates using time intervals open on the left and closed on the right (i.e. (tstart, tstop]), where the biomarker information corresponds to the start of the interval and the event (alive or dead) is recorded as the last tstop value. For this purpose, we used the tmerge function of the survival package in R (v. 3.2-13). Then, we fit a cox proportional hazard model to this data individually for each of the 28 biomarkers, including sex and breed class (small or medium). We then adjusted the p.value of each parameter to account for multiple comparison (by false discovery rate (fdr)) and selected features with an adjusted fdr below 0.05 (FIG. 1).

Using this method, we identified 13 biomarkers that are individually predictive of the survival probability in dogs:
White blood cells count (10\hat{ }3 per ul)
Serum Albumin (g/dL)
Serum Alkaline phosphatase (U/L, In-transformed)
Serum creatine Kinase (IU/L, In-transformed)
Hemoglobin (g/dL)
Hematocrit (%)
Mean Corpuscular Hemoglobin (pg)
Serum Sodium (mmol/L)
Mean Red Cell Volume (fL)
Serum Globulin (g/dL)
Serum Calcium (mg/dL)
Serum Platelet Count (10\hat{ }3/uL)
Red Blood Cell Count (10\hat{ }3/uL)

### Example 3-Multi-Parameter Model for Predicting Mortality Risk

Next, we constructed the best model that would consider multiple parameters simultaneously, as this is more likely to cover a wide range of organ dysfunctions that occur with age. However, selecting several features that might be correlated with each other is subject to bias. To avoid this issue, we used a penalized regression method using the glmnet package (v4.1-3). We fit a LASSO-penalized cox proportional hazard model on data and used 20-fold cross validation to compare different values of the penalization parameter lambda. This approach leads to the selection of the top 10 most predictive blood biomarkers for survival, by order of importance as shown below:
White blood cells count (10\hat{ }3 per ul)
Serum Albumin (g/dL)
Serum Alkaline phosphatase (U/L, In-transformed)
Serum creatine Kinase (IU/L, In-transformed)
Hemoglobin (g/dL)
Hematocrit (%)
Mean Corpuscular Hemoglobin (pg)
Serum Glucose (mg/dL)
Mean Red Cell Volume (fL)
Serum Globulin (g/dL)

We also found that the first 3 biomarkers from this list are the most predictive and that the performance can be increased by incorporating each of the next 7 biomarkers.

To extract the phenotypic age of the animal, we computed two different gompertz functions on our training set, one that models survival as a function of the selected biomarkers, age, breed class (small or medium dog) and sex (model 1) and a second function that only considers age, breed class and sex (model 2). These models were fit using the flexsurv package (v 2.1). The phenotypic age was defined as the time variable (“age”) at which the survival probability of the animal given by model 2 is equal to the survival probability at their chronological age given by the model 1. This leads to a mathematical function connecting the blood biomarkers to the phenoage and is given by the following formula:

$$Phenoage = ln ( \frac{{γ}_{breed} * {e}^{xb} * \{ {e}^{γ * age} - 1 \}}{{e}^{\{ breed * {β}_{breed 2} \} + \{ sex * {β}_{sex 2} \} + {β}_{02}} * γ} + 1 ) * \frac{1}{{γ}_{breed}}$$

Where xb is the sum of the value of each biomarkers, sex and breed multiplied by their respective coefficients. Sex and breeds are coded as numerical value with 0 for female and 1 for males and 0 for small breeds and 1 for medium breeds. The coefficients are given by the two gompertz function trained on our training sets.

As an example, the coefficients, as well as the γ and γ_{breed} values have been measured from our training set for the complete list of biomarkers and are given in Table 2.

$$xb = {\sum }_{u = 1}^{p} {x}_{u} {β}_{u} + {β}_{0}$$

Table 2-Coefficients and γ and γ_{breed} values have been measured from training set

| | Coefficient |
|---|---|
| γ | 0.491790219 |
| β₀ | −6.036261473 |
| β White blood cells count | 0.091862564 |
| β Hemoglobin | −0.009131623 |
| β Mean Red Cell Volume | −0.007486146 |
| β Hematocrit | −0.018418391 |
| β Mean Corpuscular Hemoglobin | −0.128195615 |
| β Serum Glucose | 0.009169677 |
| β Serum Globulin | 0.132755858 |
| β Serum Creatine Kinase | 0.332818902 |
| β Serum Albumin | −0.744060565 |
| β Serum Alkaline Phosphatase | 0.262594338 |
| β breed | 1.138018960 |
| β Sex | 0.151826455 |
| γ_{breed} | 0.5668399 |
| β₀₂ | −9.5204440 |
| β_{breed2} | 1.2299804 |
| βₛₑₓ₂ | 0.2678798 |

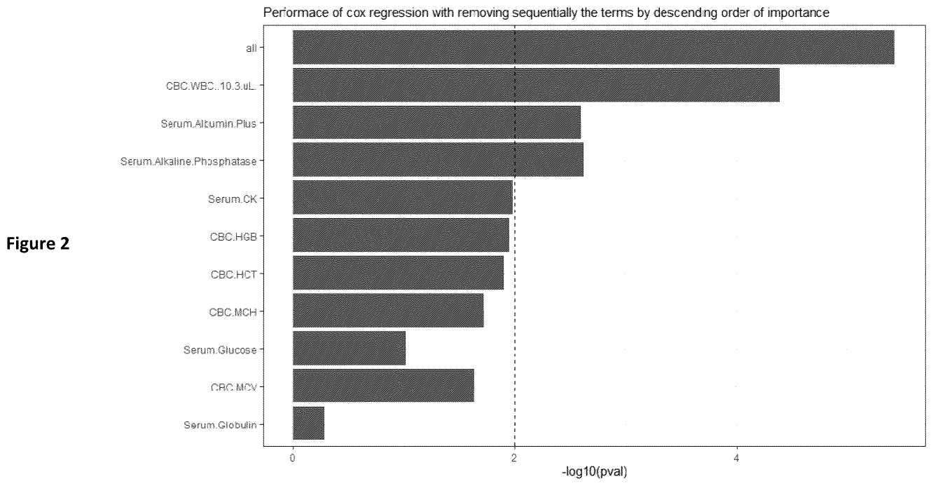

Further, by reducing the set of 10 biomarkers by systematically removing one biomarker, starting for the top of the list, we observed a reduction in the strength of the survival prediction (p value). The drop was most pronounced with the first parameters, confirming their biggest contribution, but we observed a change in quality of prediction by each reduction of the set, showing that each parameter contributes to the overall prediction (FIG. 2).

### Example 3-Generating a Biological Clock Predictive of Mortality Risk and/or Probability of a Healthy Lifespan

An elastic net regression was adjusted using phenoAge_pred (predicted value of PhenoAge at the age of DNA collection-see Example 2) as the response variable and the 12009 DNA methylation probes (see Example 1), sex and breed class as explanatory variable. The optimal lambda was 0.1177 and this selected 149 sites as forming the biological clock (see Table 3). Sex and breed class were not selected by the model.

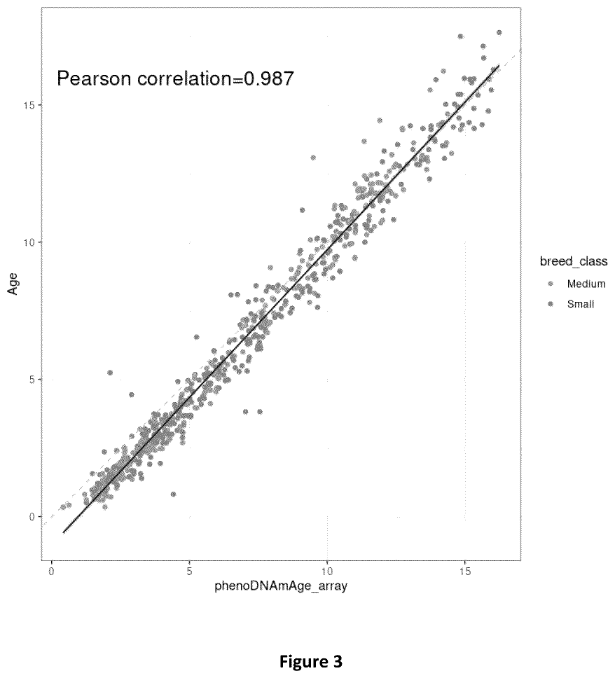

FIG. 3 shows the correlation between phenoDNAmAge (biological age according to the present biological clock) and chronological age.

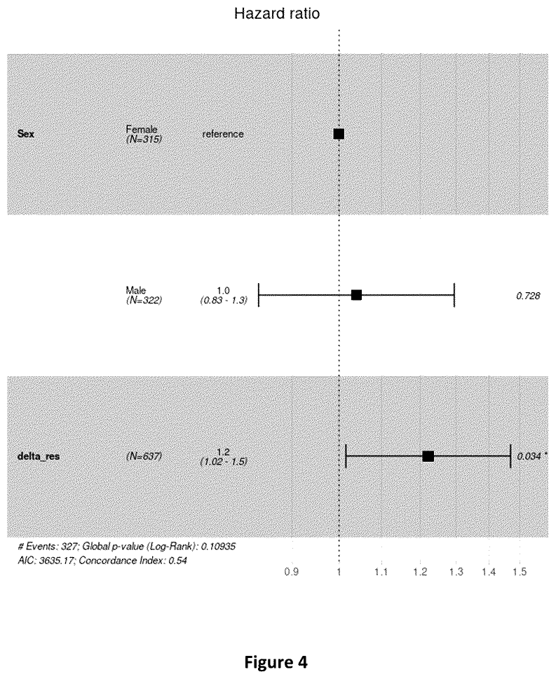

FIG. 4 shows the hazard ratio of a cox model explaining survival by sex and delta, stratified on breed class. Delta_res is obtained as the residuals of a linear model between phenoDNAmAge and chronological age. Positive values of delta indicate that the subject is biologically older than its chronological age. FIG. 4 shows that the hazard ratio is significantly bigger than 1 which indicates that subject that are biologically older have a higher mortality risk.

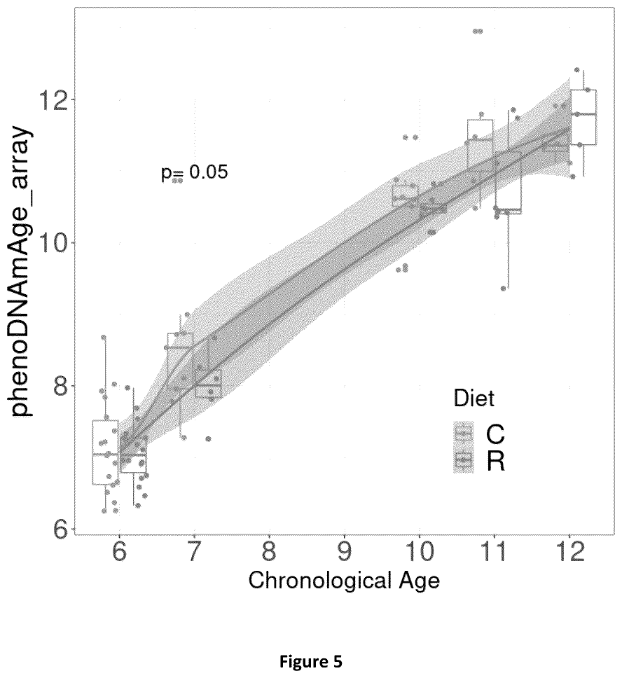

FIG. 5 shows a validation data set based on a life long calorie restriction study. FIG. 5 shows that the Calorie Restricted group (R) has consistently lower biological age than the control (C) group.

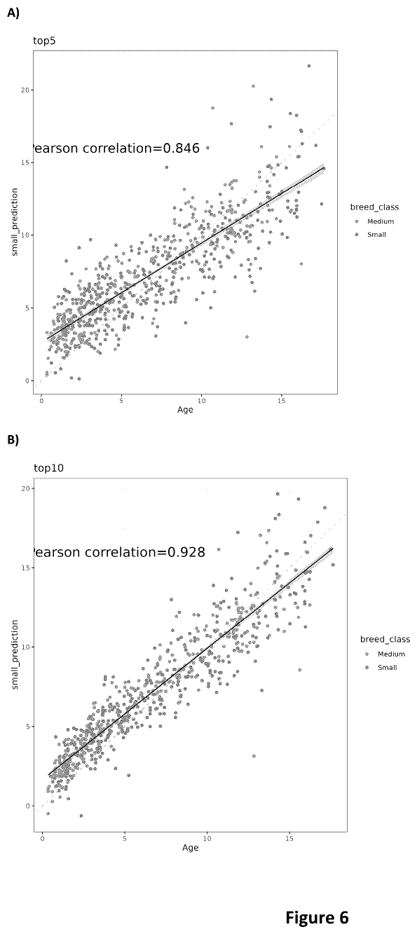

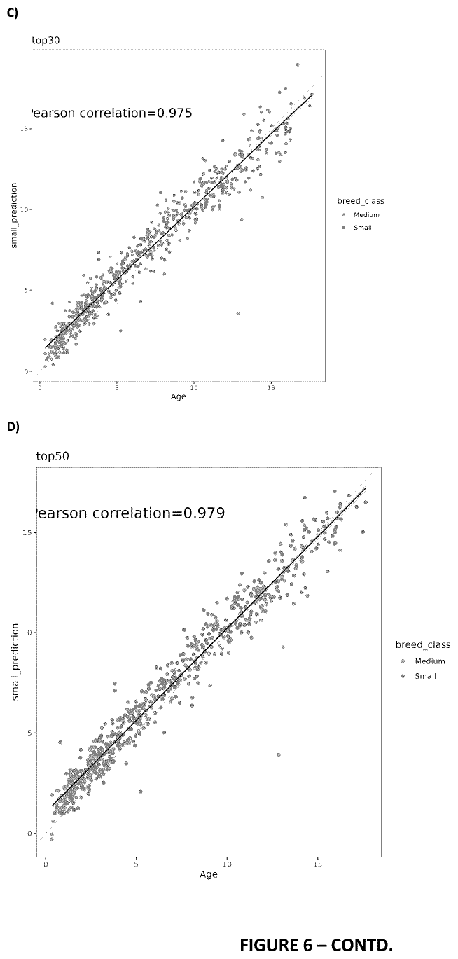

Further biological clocks were also generated using only the top 5, top 10, top 30 and top 50 sites from the complete list of sites shown in Table 3; and each was shown to correlate with biological age (see FIG. 6). These clocks were generated by selecting the top-n sites based on the absolute value of the coefficients of the full clock (in decreasing order, taking large coefficients first). A linear model explaining chronological age respectively was fitted using the topn sites as predictors. Details of the top 5, top 10, top 30 and top 50 sites clocks are shown in Tables 4-7. Phenotypic age (phenoDNAmAge) is calculated by a linear combination of the coefficients (phenoDNAmAge=Intercept+coeff*meth).

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

TABLE 3

| | | Co- | | SEQ | | | | | | |
|---|---|---|---|---|---|---|---|---|---|---|
| Site | | effi- | | ID | | probe | probe | | | |
| number | CGid | cient | Sequence | NO: | Chr | Start | End | CGstart | CGend | Strand |
| 1 | cg02471603 | >1 | CGCAATGTTCATCAA | 1 | 9 | 224702 | 22470331 | 22470282 | 22470283 | + |
| | | | GCTGAGCCGAGAAG | | | 82 | | | | |
| | | | ACATTAATAGCCTGA | | | | | | | |
| | | | TGAATA | | | | | | | |
| 2 | cg17136263 | >1 | CGAGAGCAGCTCGA | 2 | 25 | 268232 | 26823276 | 26823227 | 26823228 | + |
| | | | TGCACATCTGCCACT | | | 27 | | | | |
| | | | GCTGCAACACCTCCT | | | | | | | |
| | | | CGTGCT | | | | | | | |
| 3 | cg23194298 | >1 | TGGTTTCAAAACCGC | 3 | 36 | 201422 | 20142268 | 20142267 | 20142268 | + |
| | | | CGAATGAAACTCAA | | | 19 | | | | |
| | | | GAAGATGAGCCGAG | | | | | | | |
| | | | AGAACCG | | | | | | | |
| 4 | cg14435444 | >1 | AAGCAAATCAGATT | 4 | 37 | 232437 | 23243778 | 23243777 | 23243778 | + |
| | | | CCAGGCTGCTGCCAG | | | 29 | | | | |
| | | | GTGTTGTCTCGTCTT | | | | | | | |
| | | | CCGCCG | | | | | | | |
| 5 | cg07517669 | >1 | CGACAGGCAGGTCA | 5 | 36 | 201422 | 20142249 | 20142200 | 20142201 | + |
| | | | AGATTTGGTTTCAAA | | | 00 | | | | |
| | | | ACCGCCGAATGAAA | | | | | | | |
| | | | CTCAAGA | | | | | | | |
| 6 | cg17526723 | <−1 | CGGCACAGACTCCA | 6 | 9 | 457761 | 45776182 | 45776133 | 45776134 | + |
| | | | GGGTTCACGCATATG | | | 33 | | | | |
| | | | GCCAGACAGATGTG | | | | | | | |
| | | | TCTGGCT | | | | | | | |
| 7 | cg07330957 | >1 | CGGAGGAGCTGACC | 7 | 21 | 423349 | 42335015 | 42335014 | 42335015 | − |
| | | | AATTGGTCATGGTGC | | | 66 | | | | |
| | | | TGAAACCTTTGTGGG | | | | | | | |
| | | | GAATCG | | | | | | | |
| 8 | cg15279950 | >1 | TCCCAGCCTGGGCAA | 8 | 24 | 264135 | 26413569 | 26413568 | 26413569 | − |
| | | | AACTTTGATGATAAA | | | 20 | | | | |
| | | | TCAAGCGGCAGATC | | | | | | | |
| | | | CCTCCG | | | | | | | |
| 9 | cg04411283 | <−1 | CGAAATTAGGTGGTC | 9 | 8 | 433591 | 43359175 | 43359126 | 43359127 | + |
| | | | GTTGGAATCCTGATC | | | 26 | | | | |
| | | | GCAGTAAAGTGGTC | | | | | | | |
| | | | CTTGGT | | | | | | | |
| 10 | cg12870762 | >1 | CGTAGCCGAAGCTG | 10 | 32 | 309177 | 30917791 | 30917742 | 30917743 | − |
| | | | GAGTGCTGCTTTGCT | | | 42 | | | | |
| | | | TTCAGTCTCAGGCTG | | | | | | | |
| | | | GCCAGG | | | | | | | |
| 11 | cg04794444 | >1 | CGCTGTCCAGCTGCA | 11 | 23 | 517036 | 51703707 | 51703658 | 51703659 | − |
| | | | GCTGCGCCTGAACCT | | | 58 | | | | |
| | | | GAAAGGACAAGGGC | | | | | | | |
| | | | GTCACG | | | | | | | |
| 12 | cg09718073 | >1 | CGGTGACCCACAGG | 12 | 24 | 276233 | 27623353 | 27623304 | 27623305 | + |
| | | | TACTGCGCATGGAAC | | | 04 | | | | |
| | | | CTAATGACCAGTCAC | | | | | | | |
| | | | TCAATT | | | | | | | |
| 13 | cg00857814 | <−1 | AACCAGGTGCAAGC | 13 | 7 | 165441 | 16544157 | 16544156 | 16544157 | − |
| | | | TGGGAAACAGTCCC | | | 08 | | | | |
| | | | ACATTCCTTACAGCC | | | | | | | |
| | | | AGCAACG | | | | | | | |
| 14 | cg13804575 | <−1 | CGTCTGGGATGGGG | 14 | 11 | 620677 | 62067776 | 62067727 | 62067728 | + |
| | | | AAAGACCAACCAGT | | | 27 | | | | |
| | | | TGGGGCTTTCTCCCA | | | | | | | |
| | | | GGGCTCC | | | | | | | |
| 15 | cg20173540 | >1 | ACAGATGCAAATAC | 15 | 28 | 242467 | 24246803 | 24246802 | 24246803 | + |
| | | | TCCAAAGAAGTGTC | | | 54 | | | | |
| | | | GGGCACTGTTCGGGC | | | | | | | |
| | | | TTGACCG | | | | | | | |
| 16 | cg04607114 | <−1 | AAGTCCGAGAGGGG | 16 | 35 | 201260 | 2012655 | 2012654 | 2012655 | − |
| | | | GCCTTTCACATGACA | | | 6 | | | | |
| | | | TCATAAAAGCCTGAT | | | | | | | |
| | | | TTATCG | | | | | | | |
| 17 | cg23899560 | <−1 | CGAGTTTGGGGGCAT | 17 | 12 | 569387 | 56938758 | 56938709 | 56938710 | + |
| | | | GACAGTACATCTGAC | | | 09 | | | | |
| | | | CCCTTTGGGGTAAAA | | | | | | | |
| | | | TTTGA | | | | | | | |
| 18 | cg21345677 | <−1 | ATGGCCAGTCATTTT | 18 | 26 | 111582 | 11158263 | 11158262 | 11158263 | − |
| | | | GTTCACAACTTTGCA | | | 14 | | | | |
| | | | GCAAGCAGGAGCAA | | | | | | | |
| | | | AAGCCG | | | | | | | |
| 19 | cg18737166 | <−1 | TAAACTCCTATGTAT | 19 | 16 | 470876 | 47087720 | 47087719 | 47087720 | + |
| | | | GTTCACATCTATGAT | | | 71 | | | | |
| | | | CTGCTAACCATTGCT | | | | | | | |
| | | | ACTCG | | | | | | | |
| 20 | cg23177112 | >1 | CGGATATAATTATCG | 20 | 36 | 167019 | 16701969 | 16701920 | 16701921 | + |
| | | | AGGAGTCAAGATGT | | | 20 | | | | |
| | | | TATGCTAAAAACTAT | | | | | | | |
| | | | GCATTG | | | | | | | |
| 21 | cg00038955 | >1 | GTCTTCCTTTCCTGT | 21 | 13 | 150688 | 150693 | 1506932 | 1506933 | − |
| | | | ACTGACAAGCTGAA | | | 4 | | | | |
| | | | CAGACGCACCTTGTT | | | | | | | |
| | | | GGTTCG | | | | | | | |
| 22 | cg10196526 | >1 | CGTGTCTGTTTGCAG | 22 | 20 | 283840 | 28384084 | 28384035 | 28384036 | + |
| | | | CACCCCTGGGGCGA | | | 35 | | | | |
| | | | GCTGTGGCTTCCTGT | | | | | | | |
| | | | AACATG | | | | | | | |
| 23 | cg01213012 | >1 | CGAAGGTGAACTTCT | 23 | 27 | 383895 | 38389639 | 38389590 | 38389591 | − |
| | | | TGATCTGGATGACCA | | | 90 | | | | |
| | | | GGTAGTCAGGGAAT | | | | | | | |
| | | | GAGGCA | | | | | | | |
| 24 | cg19317509 | >1 | CGGATCTGAGCACTT | 24 | 36 | 166919 | 16691972 | 16691923 | 16691924 | + |
| | | | GAGACTACCATTTAA | | | 23 | | | | |
| | | | TCAAATGAATCAGTA | | | | | | | |
| | | | ACTAA | | | | | | | |
| 25 | cg27637204 | <−1 | CGCCATCCCAGGTGG | 25 | 30 | 252982 | 2529869 | 2529820 | 2529821 | + |
| | | | TTGAGTTCAGCCAGG | | | 0 | | | | |
| | | | TTGAGCACAACACA | | | | | | | |
| | | | GATGCC | | | | | | | |
| 26 | cg22094235 | <−1 | GTGGAAGGCGGCGT | 26 | 31 | 294273 | 29427360 | 29427359 | 29427360 | + |
| | | | GAAGCGGCGGCTCG | | | 11 | | | | |
| | | | TGCTGGCATCTACGG | | | | | | | |
| | | | GGATACG | | | | | | | |
| 27 | cg18287975 | >1 | CGCGTACACCCAGA | 27 | 3 | 196859 | 19686006 | 19685957 | 19685958 | − |
| | | | CATCTTCGGGCTGCT | | | 57 | | | | |
| | | | ATTGGATTGACTTTG | | | | | | | |
| | | | AAGGTT | | | | | | | |
| 28 | cg15486794 | <−1 | GGGCCAGTGTCGGG | 28 | 18 | 517878 | 51787855 | 51787854 | 51787855 | + |
| | | | ACAGTTGTCAGCAG | | | 06 | | | | |
| | | | GGCTCCAACATGAC | | | | | | | |
| | | | GCTCATCG | | | | | | | |
| 29 | cg21508838 | >1 | CCATGTACTTGAGAT | 29 | 3 | 198836 | 19883698 | 19883697 | 19883698 | − |
| | | | TTCTCATGACATCAT | | | 49 | | | | |
| | | | CATTACCTTGGTCTC | | | | | | | |
| | | | CCGCG | | | | | | | |
| 30 | cg00312241 | >1 | TCAGCCAGCAGAGA | 30 | 20 | 405021 | 40502242 | 40502241 | 40502242 | − |
| | | | GATTGCAGCCTTCTT | | | 93 | | | | |
| | | | CCCTGACTTCAGTGA | | | | | | | |
| | | | ACAGCG | | | | | | | |
| 31 | cg17356865 | <−1 | AAAATAAAACTCAG | 31 | 12 | 117734 | 11773473 | 11773472 | 11773473 | − |
| | | | GGACCAACATCTGCT | | | 24 | | | | |
| | | | TCCTTTGCACTTGCT | | | | | | | |
| | | | CCGTCG | | | | | | | |
| 32 | cg22419039 | <−1 | CGCACTCCCTGTGAC | 32 | 38 | 124319 | 12431950 | 12431901 | 12431902 | − |
| | | | CCAGGACGTCATGAT | | | 01 | | | | |
| | | | GCCTGTCTGTTTTCT | | | | | | | |
| | | | CAATG | | | | | | | |
| 33 | cg23852530 | >1 | CGAAGTAAAAAATG | 33 | 8 | 411284 | 41128540 | 41128491 | 41128492 | + |
| | | | TTGATCTTGCATCAC | | | 91 | | | | |
| | | | CAGAGGAACATCAG | | | | | | | |
| | | | AAGCACC | | | | | | | |
| 34 | cg01961426 | 0 < x | CGCAGGGAGAGATT | 34 | 3 | 444676 | 44467725 | 44467676 | 44467677 | − |
| | | < 1 | AAGATCTCGTTGAAA | | | 76 | | | | |
| | | | AGGAATAAAAATAA | | | | | | | |
| | | | CATCATC | | | | | | | |
| 35 | cg18824846 | 0 < x | CGACGTGTCCATCCT | 35 | 8 | 388111 | 38811192 | 38811143 | 38811144 | + |
| | | < 1 | GACCATCGAGGATG | | | 43 | | | | |
| | | | GCATCTTCGAGGTGA | | | | | | | |
| | | | AGTCCA | | | | | | | |
| 36 | cg02691325 | 0 < x | CGAGTGGCACATCC | 36 | 12 | 126949 | 12694976 | 12694927 | 12694928 | + |
| | | <1 | GCTCCAACCAGCAG | | | 27 | | | | |
| | | | CTGGTGCCCAGCTAC | | | | | | | |
| | | | TCTGAGG | | | | | | | |
| 37 | cg06363517 | 0 <x | GGTCATCCACCTGCT | 37 | 25 | 268238 | 26823858 | 26823857 | 26823858 | − |
| | | < 1 | GCAGATGGGGCAGG | | | 09 | | | | |
| | | | TGTGGAGGTAAGAG | | | | | | | |
| | | | CACTGCG | | | | | | | |
| 38 | cg08476750 | - | TTAGCACAGTTTAAC | 38 | 2 | 324453 | 32445372 | 32445371 | 32445372 | + |
| | | 1<x< | TCCACCCTCATTTAA | | | 23 | | | | |
| | | 0 | ACTTCCTTTGATTCT | | | | | | | |
| | | | TTCCG | | | | | | | |
| 39 | cg25774993 | 0 <x | CTGTCTTCCCACAGG | 39 | 39 | 109134 | 109134 | 1.09E+ | 10913 | − |
| | | <1 | GCTCCATTGTGTGCA | | | 934 | 983 | 08 | 4983 | |
| | | | GTTCCTGTTTCTCAG | | | | | | | |
| | | | GGGCG | | | | | | | |
| 40 | cg26245113 | − | CGAAAGCCGTGTGA | 40 | 27 | 120513 | 120518 | 120518 | 12051 | + |
| | | 1 < | ACTCTTGGTGAACCA | | | 8 | 7 | 6 | 87 | |
| | | x < 0 | AGATTGAAGTCATA | | | | | | | |
| | | | AATCACG | | | | | | | |
| 41 | cg02941993 | 0 < x | CGCTGCATCGCTGCT | 41 | 28 | 387200 | 387200 | 387200 | 38720 | − |
| | | < 1 | CTAGGGAAGAGGTT | | | 49 | 98 | 49 | 050 | |
| | | | AACTGACAGAATCA | | | | | | | |
| | | | CAATCCA | | | | | | | |
| 42 | cg27153217 | − | TTCAGACCTTCTCAA | 42 | 29 | 411605 | 411610 | 411610 | 41161 | − |
| | | 1 < x | TATGATTCACATTTG | | | 2 | 1 | 0 | 01 | |
| | | < 0 | CACATCAACAGCCTC | | | | | | | |
| | | | ATGCG | | | | | | | |
| 43 | cg17691933 | 0 < x | CGAGTAATGAAATA | 43 | 36 | 206392 | 206393 | 206392 | 20639 | |
| | | < 1 | ATCATGTCCAGAAAT | | | 74 | 23 | 74 | 275 | |
| | | | GTATCAAAGGCCAG | | | | | | | |
| | | | AGGGATT | | | | | | | |
| 44 | cg16995667 | 0 < x | TGTGGTTTCCCCGTG | 44 | 17 | 288344 | 288344 | 288344 | 28834 | + |
| | | < 1 | TGTGAGGTGGGATCC | | | 30 | 79 | 78 | 479 | |
| | | | ACTCCCCGCATAGTC | | | | | | | |
| | | | TCTCG | | | | | | | |
| 45 | cg07116727 | 0 < x | CAATGCTTGAAGGA | 45 | 6 | 155957 | 155958 | 155958 | 15595 | − |
| | | < 1 | GCCCAAATCAGGAG | | | 78 | 27 | 26 | 827 | |
| | | | TCTAATTGTAATCAG | | | | | | | |
| | | | GAAATCG | | | | | | | |
| 46 | cg00930337 | − | ACGAGCTGCATGCAT | 46 | 35 | 136717 | 136718 | 136718 | 13671 | − |
| | | 1 < x | GCCAAATCAGGTCAT | | | 77 | 26 | 25 | 826 | |
| | | < 0 | TCACAAATATAGAA | | | | | | | |
| | | | CAAACG | | | | | | | |
| 47 | cg06912074 | 0 < x | TGTTAATTTTTCCAT | 47 | 23 | 262958 | 262959 | 262959 | 26295 | − |
| | | < 1 | CTGCTTTGGCTGCAG | | | 85 | 34 | 33 | 934 | |
| | | | GTAATTTGGAGACAC | | | | | | | |
| | | | TGACG | | | | | | | |
| 48 | cg06177598 | − | AAAATGCATCTTGTT | 48 | 37 | 823464 | 823469 | 823469 | 82346 | − |
| | | 1 < x | TCTTTCAACCCTTGA | | | 3 | 2 | 1 | 92 | |
| | | < 0 | CTGTTTTGACATTTT | | | | | | | |
| | | | CTTCG | | | | | | | |
| 49 | cg19437183 | 0 < x | TTCTTGCCTCTGAGG | 49 | 29 | 248465 | 248465 | 248465 | 24846 | − |
| | | < 1 | AGCTGCCCAATGACT | | | 46 | 95 | 94 | 595 | |
| | | | GGAGGTCTGGGATT | | | | | | | |
| | | | AAAGCG | | | | | | | |
| 50 | cg26352755 | 0 < x | GGAGTGCTGCTTTGC | 50 | 32 | 309177 | 309178 | 309178 | 30917 | − |
| | | < 1 | TTTCAGTCTCAGGCT | | | 55 | 04 | 03 | 804 | |
| | | | GGCCAGGCTCGAGTT | | | | | | | |
| | | | ACACG | | | | | | | |
| 51 | cg09105275 | − | CGTTGTGTGGTGTGC | 51 | 29 | 411112 | 411116 | 411112 | 41111 | + |
| | | 1 < x | AGGGACACTCTGTG | | | 0 | 9 | 0 | 21 | |
| | | < 0 | ATACTCTAGTGAGCT | | | | | | | |
| | | | GCTAAG | | | | | | | |
| 52 | cg15642312 | − | CGCCTCTGCTCAGTC | 52 | 34 | 171445 | 171445 | 171445 | 17144 | − |
| | | 1 < x | CTCAAGCCCTGGGCG | | | 09 | 58 | 09 | 510 | |
| | | < 0 | TGGTGCCCAGAATA | | | | | | | |
| | | | GGGTGC | | | | | | | |
| 53 | cg07925213 | − | CGCTGTTGCCCTTGG | 53 | 3 | 616100 | 616100 | 616100 | 61610 | − |
| | | 1 < x | GAGCCATGGAAAGG | | | 18 | 67 | 18 | 019 | |
| | | < 0 | GCCAATTTGCTCGCA | | | | | | | |
| | | | GTCTTA | | | | | | | |
| 54 | cg13787598 | − | ATCTTGTTCAGCCAT | 54 | 5 | 137482 | 137483 | 137483 | 13748 | + |
| | | 1 < x | GTGTCGCCTGCCACA | | | 77 | 26 | 25 | 326 | |
| | | < 0 | AACTGCAAAACAGA | | | | | | | |
| | | | ACAACG | | | | | | | |
| 55 | cg01151827 | − | CGTAATGTAACTGAA | 55 | 20 | 234407 | 234407 | 234407 | 23440 | − |
| | | 1 < x | AATTATTGAAGTGAG | | | 35 | 84 | 35 | 736 | |
| | | < 0 | AAAAAAGAGAAACA | | | | | | | |
| | | | GGGAAG | | | | | | | |
| 56 | cg24837930 | − | GTCAATGTCAGCAA | 56 | 7 | 573855 | 573855 | 573855 | 57385 | − |
| | | 1 < x | ATGTCCACTTACTGG | | | 29 | 78 | 77 | 578 | |
| | | < 0 | ATTGAAGGTTGTTTC | | | | | | | |
| | | | TAAACG | | | | | | | |
| 57 | cg03682073 | 0 < x | CCAGTCCTGTCTCCT | 57 | 24 | 332874 | 332875 | 332875 | 33287 | + |
| | | < 1 | CTGAGGGCATCAAG | | | 80 | 29 | 28 | 529 | |
| | | | GACTTCTTCAGCATG | | | | | | | |
| | | | AAGCCG | | | | | | | |
| 58 | cg10452170 | 0 < x | CGAGCCCCGCCGGC | 58 | 1 | 107519 | 107520 | 1.08E+ | 10751 | + |
| | | < 1 | CATCCTCGCTGATGG | | | 966 | 015 | 08 | 9967 | |
| | | | TGGGCATCACCTCGC | | | | | | | |
| | | | ACATCA | | | | | | | |
| 59 | cg02520894 | 0 < x | CGCCAGCTTGTCCAC | 59 | 10 | 269893 | 269894 | 269893 | 26989 | + |
| | | < 1 | CTCGCGCTCCAGCTC | | | 51 | 00 | 51 | 352 | |
| | | | CGACTTCTGCTTCTG | | | | | | | |
| | | | CAGCT | | | | | | | |
| 60 | cg24534944 | − | CGATTAGGCAGAAA | 60 | 3 | 675353 | 675353 | 675353 | 67535 | − |
| | | 1 < x | TGAAGGAGATCATA | | | 32 | 81 | 32 | 333 | |
| | | < 0 | AAGGTGAGAGATTT | | | | | | | |
| | | | CTCCACAA | | | | | | | |
| 61 | cg12818872 | 0< x | CGTAAAGGTCAAGC | 61 | 10 | 469299 | 469300 | 469299 | 46929 | − |
| | | < 1 | ATTGTGACATACCTT | | | 74 | 23 | 74 | 975 | |
| | | | TCAAATAATCCCGCC | | | | | | | |
| | | | TAACTT | | | | | | | |
| 62 | cg14002474 | − | CGAGATAATCTTTTA | 62 | 32 | 256570 | 256571 | 256570 | 25657 | + |
| | | 1 < x | AGGTGCACTGTTAAG | | | 70 | 19 | 70 | 071 | |
| | | < 0 | GTGGAACCTAAATGT | | | | | | | |
| | | | TGCTG | | | | | | | |
| 63 | cg16157644 | 0 < x | CGGGAAAGTGGCTTT | 63 | 4 | 262534 | 262534 | 262534 | 26253 | + |
| | | < 1 | AATACCCTGAAAAG | | | 18 | 67 | 18 | 419 | |
| | | | CAAAGGAATCCGCC | | | | | | | |
| | | | TGTCAGC | | | | | | | |
| 64 | cg23263484 | 0 < x | TTGTGGGGAATCGTG | 64 | 21 | 423350 | 423350 | 423350 | 42335 | − |
| | | < 1 | GCCAAGTGCACTGA | | | 03 | 52 | 51 | 052 | |
| | | | CTCTGTGGGAAAAC | | | | | | | |
| | | | GGCGGCG | | | | | | | |
| 65 | cg10450430 | 0 < x | CGCTGGCGTAGCCG | 65 | 32 | 309177 | 309177 | 309177 | 30917 | − |
| | | < 1 | AAGCTGGAGTGCTG | | | 36 | 85 | 36 | 737 | |
| | | | CTTTGCTTTCAGTCT | | | | | | | |
| | | | CAGGCTG | | | | | | | |
| 66 | cg22239755 | 0 < x | CGCTGGGTAATGGCC | 66 | 24 | 275179 | 275180 | 275179 | 27517 | + |
| | | < 1 | CCTGTAAAGTGTTAA | | | 66 | 15 | 66 | 967 | |
| | | | TTGCCTATTGAGACT | | | | | | | |
| | | | GCAGA | | | | | | | |
| 67 | cg11070690 | − | TAATCAATCTTGCAG | 67 | 28 | 137787 | 137788 | 137788 | 13778 | − |
| | | 1 < x | CTGTCAGGCCGACA | | | 65 | 14 | 13 | 814 | |
| | | < 0 | GGCAGGAGTATTAA | | | | | | | |
| | | | CCTGGCG | | | | | | | |
| 68 | cg05897263 | 0< x | CGGCTTTTTGAATGA | 68 | 14 | 371661 | 371662 | 371661 | 37166 | + |
| | | < 1 | GCCACGTGTTCAAAC | | | 52 | 01 | 52 | 153 | |
| | | | TACAAATCAACGTCT | | | | | | | |
| | | | CACGT | | | | | | | |
| 69 | cg14212735 | 0< x | AATATGGCTCGTCCT | 69 | 5 | 179494 | 179495 | 179495 | 17949 | + |
| | | < 1 | TCAATTTGATGCTTG | | | 68 | 17 | 16 | 517 | |
| | | | AGCAACAGAGGGTC | | | | | | | |
| | | | AGAGCG | | | | | | | |
| 70 | cg02034779 | 0 < x | GGCACACCAGCTGC | 70 | 2 | 758031 | 758031 | 758031 | 75803 | + |
| | | < 1 | CTGTTTTGCATGGTA | | | 27 | 76 | 75 | 176 | |
| | | | TTTGCAAAAATGCCT | | | | | | | |
| | | | CTTGCG | | | | | | | |
| 71 | cg25323201 | − | ACAATAGATCTGAG | 71 | 34 | 340332 | 340332 | 340332 | 34033 | − |
| | | 1<x< | CAGACAGATGAAAT | | | 38 | 87 | 86 | 287 | |
| | | 0 | TTAAAACTTCAGCAT | | | | | | | |
| | | | GAGTGCG | | | | | | | |
| 72 | cg00106809 | 0 < x | CGTATTTTCTGACAA | 72 | 13 | 393727 | 393732 | 393727 | 39372 | − |
| | | < 1 | TATGGAAGAATTCA | | | 6 | 5 | 6 | 77 | |
| | | | AGGATGATGTAATTT | | | | | | | |
| | | | CCTCTT | | | | | | | |
| 73 | cg08464821 | 0 < x | GGGGGATCGAGCGT | 73 | 25 | 398724 | 398725 | 398725 | 39872 | − |
| | | < 1 | TTGGGGGCGCTCGAC | | | 95 | 44 | 43 | 544 | |
| | | | TTGTGCCGCCACTTC | | | | | | | |
| | | | TTCTCG | | | | | | | |
| 74 | cg13424698 | 0 < x | CAAATTATAGAGTTC | 74 | 3 | 440660 | 440660 | 440660 | 44066 | + |
| | | < 1 | AGCTTCAACACACGC | | | 37 | 86 | 85 | 086 | |
| | | | TCTGTCACCCGAGAT | | | | | | | |
| | | | CAGCG | | | | | | | |
| 75 | cg04265576 | − | CGCGCCCCTCTGCAG | 75 | 14 | 403744 | 403745 | 403744 | 40374 | − |
| | | 1 < x | GACTGTGATTTGTTG | | | 71 | 20 | 71 | 472 | |
| | | < 0 | TGTATTAGTACATCT | | | | | | | |
| | | | GGCTA | | | | | | | |
| 76 | cg01255766 | − | TCTTCTGCTCGTGCA | 76 | 30 | 359671 | 359672 | 359672 | 35967 | + |
| | | 1 < x | GTGCACTCTGGGCCT | | | 97 | 46 | 45 | 246 | |
| | | < 0 | TGAGAGCAGAGTCC | | | | | | | |
| | | | CGGGCG | | | | | | | |
| 77 | cg19705440 | − | TACTGTACTAATTAT | 77 | 30 | 320580 | 320581 | 320581 | 32058 | + |
| | | 1 < x | GTAAATTAAACCTAA | | | 56 | 05 | 04 | 105 | |
| | | < 0 | TTAACTGACGAAAA | | | | | | | |
| | | | CTGCCG | | | | | | | |
| 78 | cg02251239 | 0 < x | CGGTCTACCAGAAA | 78 | 22 | 319754 | 319754 | 319754 | 31975 | + |
| | | < 1 | GCTAGCCCAGTTTAG | | | 12 | 61 | 12 | 413 | |
| | | | TGCTCAGTTTCAAAT | | | | | | | |
| | | | GCATAG | | | | | | | |
| 79 | cg24515358 | 0 < x | CGTTGGAGAGCAAC | 79 | 31 | 133021 | 133021 | 133021 | 13302 | + |
| | | < 1 | TAAAATCTGACTGAT | | | 23 | 72 | 23 | 124 | |
| | | | TTCCATCTTTGGAGC | | | | | | | |
| | | | ATCAGA | | | | | | | |
| 80 | cg13605024 | − | TTGTGGTGTAATCAA | 80 | 2 | 588518 | 588518 | 588518 | 58851 | + |
| | | 1 < x | CTTGCCATACATGCA | | | 48 | 97 | 96 | 897 | |
| | | < 0 | TTACCTCCTAATGAG | | | | | | | |
| | | | CGCCG | | | | | | | |
| 81 | cg22851118 | − | CGTGAAAGAAAATA | 81 | 7 | 510263 | 510263 | 510263 | 51026 | − |
| | | 1 < x | TGAATCTAATTTAAA | | | 37 | 86 | 37 | 338 | |
| | | < 0 | TTCAAACTGGATTTG | | | | | | | |
| | | | GGATAT | | | | | | | |
| 82 | cg10763467 | 0 < x | GCATTACTCGCAGTC | 82 | 36 | 200981 | 200981 | 200981 | 20098 | − |
| | | < 1 | AGCTAAATGAAACA | | | 18 | 67 | 66 | 167 | |
| | | | TTATTCTAAACATAT | | | | | | | |
| | | | GCATCG | | | | | | | |
| 83 | cg07853634 | 0 < x | ACACAATGGCAGGT | 83 | 5 | 175184 | 175184 | 175184 | 17518 | + |
| | | < 1 | TCCTTGACAATGTCA | | | 10 | 59 | 58 | 459 | |
| | | | TGCGTCTATTCAAAG | | | | | | | |
| | | | CAACCG | | | | | | | |
| 84 | cg10501210 | − | CGCCTGCGCAGACCC | 84 | 7 | 629439 | 629444 | 629439 | 62944 | + |
| | | 1 < x | AAATCTTGGTCCCGC | | | 9 | 8 | 9 | 00 | |
| | | < 0 | CGTAAGGTGCCGCA | | | | | | | |
| | | | GTCCCG | | | | | | | |
| 85 | cg17942396 | 0 < x | CGTTAGTAATGGAA | 85 | 4 | 381883 | 381883 | 381883 | 38188 | + |
| | | < 1 | AATACCAGGTTGTTT | | | 00 | 49 | 00 | 301 | |
| | | | AAAATTATAATAATA | | | | | | | |
| | | | ATTGTT | | | | | | | |
| 86 | cg26044837 | − | CGCCCTGCATGTGTC | 86 | 8 | 863179 | 863184 | 863179 | 86317 | + |
| | | 1 < x | TGGGCTCCCCTGGCC | | | 7 | 6 | 7 | 98 | |
| | | < 0 | AGTCCGTTTTTTTGT | | | | | | | |
| | | | CTCTG | | | | | | | |
| 87 | cg20971724 | − | TTGTCTCAAATCAGC | 87 | 2 | 554319 | 554319 | 554319 | 55431 | + |
| | | 1 < x | TACCTGGTGGACAAT | | | 14 | 63 | 62 | 963 | |
| | | < 0 | TTAACCAAGAAAAA | | | | | | | |
| | | | TTACCG | | | | | | | |
| 88 | cg14104252 | − | TTGGAATCACAAAGT | 88 | 11 | 674744 | 674744 | 674744 | 67474 | + |
| | | 1 < x | GGCCCATGGCGGAG | | | 40 | 89 | 88 | 489 | |
| | | < 0 | AATGCAGCCAGAAC | | | | | | | |
| | | | AAAGGCG | | | | | | | |
| 89 | cg08193095 | − | CGTCGGGATGTTTGG | 89 | 7 | 451485 | 451490 | 451485 | 45148 | − |
| | | 1 < x | CTGTAATGCCCCAAG | | | 2 | 1 | 2 | 53 | |
| | | < 0 | ATTTGTTCTCCCTGA | | | | | | | |
| | | | AAAAA | | | | | | | |
| 90 | cg02175825 | − | CGAGCCCTGCTTTCA | 90 | 12 | 564364 | 564364 | 564364 | 56436 | + |
| | | 1 < x | GTAATTTGCTGTAAA | | | 10 | 59 | 10 | 411 | |
| | | < 0 | CTCAGGGGAGGCTG | | | | | | | |
| | | | GCGCTA | | | | | | | |
| 91 | cg24956561 | − | CCGCTGACTTCTCTG | 91 | 14 | 548353 | 548354 | 548354 | 54835 | − |
| | | 1 < x | ATCAACACATAATTA | | | 65 | 14 | 13 | 414 | |
| | | < 0 | TCTCTGAATAAAAAT | | | | | | | |
| | | | GCACG | | | | | | | |
| 92 | cg05422546 | 0 < x | CGAGAACCGAACTT | 92 | 21 | 248164 | 248165 | 248164 | 24816 | − |
| | | < 1 | ACCAAGCATCCTCTG | | | 52 | 01 | 52 | 453 | |
| | | | CGGCTTTCAGTAAAT | | | | | | | |
| | | | ACAGCC | | | | | | | |
| 93 | cg08535072 | − | ATTCCAACACTGAAA | 93 | 11 | 344094 | 344095 | 344095 | 34409 | − |
| | | 1 < x | CAGCACCATTCCAAA | | | 90 | 39 | 38 | 539 | |
| | | < 0 | AGTGGTAACTAGAG | | | | | | | |
| | | | AAACCG | | | | | | | |
| 94 | cg03834148 | − | GCCACACAGAGAAG | 94 | 38 | 186053 | 186054 | 186054 | 18605 | + |
| | | 1 < x | ATAGCCGCTGACGG | | | 71 | 20 | 19 | 420 | |
| | | < 0 | ACACTTCATTTTAAT | | | | | | | |
| | | | TGTAACG | | | | | | | |
| 95 | cg09219505 | 0 < x | CGCGTCCAAGGCTGC | 95 | 36 | 200959 | 200959 | 200959 | 20095 | − |
| | | < 1 | TGCTTAATCCAATGA | | | 08 | 57 | 08 | 909 | |
| | | | AGGCAATTTCCGAG | | | | | | | |
| | | | GATAAT | | | | | | | |
| 96 | cg24805210 | − | AATAAATAATATTCT | 96 | 8 | 317834 | 317839 | 317839 | 31783 | − |
| | | 1 < x | GCACATCAAATCACT | | | 9 | 8 | 7 | 98 | |
| | | < 0 | TTCACCGGCCCCCAC | | | | | | | |
| | | | CCCCG | | | | | | | |
| 97 | cg17469509 | − | CGTGGATGGGAATTT | 97 | 32 | 256569 | 256570 | 256569 | 25656 | − |
| | | 1 < x | CTAATAGATCTGCCT | | | 74 | 23 | 74 | 975 | |
| | | < 0 | GGCCCTGTGCTGCTT | | | | | | | |
| | | | TTCAA | | | | | | | |
| 98 | cg21456069 | 0 < x | CATAGTTTACAGCTG | 98 | 10 | 398013 | 398013 | 398013 | 39801 | + |
| | | < 1 | TATCCGCTTTCCACA | | | 10 | 59 | 58 | 359 | |
| | | | CGTGGCAAATGATTG | | | | | | | |
| | | | CCTCG | | | | | | | |
| 99 | cg11342997 | 0 < x | GGCCTTCATCGTGTG | 99 | 20 | 911175 | 911179 | 911179 | 91117 | + |
| | | < 1 | CTGGACGCCTTTCTT | | | 0 | 9 | 8 | 99 | |
| | | | CTTCGTGCAGATGTG | | | | | | | |
| | | | GAGCG | | | | | | | |
| 100 | cg05037556 | 0 < x | AGCCCTACTGTGTTG | 100 | 10 | 477675 | 477676 | 477676 | 47767 | + |
| | | < 1 | GAATAGAGCGTAAC | | | 52 | 01 | 00 | 601 | |
| | | | CAGCTGGAGGACTG | | | | | | | |
| | | | TAAGACG | | | | | | | |
| 101 | cg05128975 | 0 < x | CACCCACAACACAA | 101 | 2 | 384012 | 384012 | 384012 | 38401 | + |
| | | < 1 | GATCAGACCCCAAG | | | 08 | 57 | 56 | 257 | |
| | | | TTCTGCTGTTTCAGT | | | | | | | |
| | | | TGCCACG | | | | | | | |
| 102 | cg16295770 | − | CACCCCATGGACCA | 102 | 16 | 153829 | 153830 | 153830 | 15383 | + |
| | | 1 < x | GAAACTCAAAAAGT | | | 74 | 23 | 22 | 023 | |
| | | < 0 | TTGCTTTTCGTGGCT | | | | | | | |
| | | | TTGCGCG | | | | | | | |
| 103 | cg10839671 | 0 < x | CGGCTTTGTTGCCAA | 103 | 32 | 819591 | 819596 | 819591 | 81959 | − |
| | | < 1 | TTTCATGCCATGTAT | | | 3 | 2 | 3 | 14 | |
| | | | TGCTCCATGTTTTGT | | | | | | | |
| | | | GCTTC | | | | | | | |
| 104 | cg24664689 | 0 < x | CGATGCACATCTGCC | 104 | 25 | 268232 | 268232 | 268232 | 26823 | + |
| | | < 1 | ACTGCTGCAACACCT | | | 38 | 87 | 38 | 239 | |
| | | | CCTCGTGCTACTGGG | | | | | | | |
| | | | GCTGC | | | | | | | |
| 105 | cg24399485 | 0 < x | CGAAAGTATTGTGTT | 105 | 1 | 713763 | 713764 | 713763 | 71376 | + |
| | | < 1 | CCAGCTGCAGGTCA | | | 99 | 48 | 99 | 400 | |
| | | | GGGCCGCCAAAGCT | | | | | | | |
| | | | TACCTCC | | | | | | | |
| 106 | cg23777581 | 0 < x | TGGGCGGGGTAGCC | 106 | 8 | 359395 | 359400 | 359400 | 35940 | − |
| | | < 1 | CAACATGTGGACGT | | | 7 | 6 | 5 | 06 | |
| | | | AGAGCAGTTTGGCC | | | | | | | |
| | | | AGCTGCCG | | | | | | | |
| 107 | cg02002684 | − | CCGTGCTGATTGGTT | 107 | 16 | 144608 | 144608 | 144608 | 14460 | − |
| | | 1 < x | TCATCCATTTTATTG | | | 31 | 80 | 79 | 880 | |
| | | < 0 | TCAAGGAAATTAAC | | | | | | | |
| | | | AGCCCG | | | | | | | |
| 108 | cg07169347 | 0 < x | CTATGCCCAGAAACT | 108 | 7 | 427056 | 427056 | 427056 | 42705 | + |
| | | < 1 | GAAGTACAAGGCCA | | | 15 | 64 | 63 | 664 | |
| | | | TTAGCGAGGAGCTG | | | | | | | |
| | | | GACCACG | | | | | | | |
| 109 | cg26799881 | − | CGTCCCCTGTAACGT | 109 | 22 | 363354 | 363354 | 363354 | 36335 | + |
| | | 1 < x | TTCCAGCGGCAAAA | | | 10 | 59 | 10 | 411 | |
| | | < 0 | CAAAGAGACGTCTC | | | | | | | |
| | | | CAGCAAC | | | | | | | |
| 110 | cg01334218 | − | CGCTGGGGTCTGCCC | 110 | 8 | 115755 | 115756 | 115755 | 11575 | + |
| | | 1 < x | CTTGGGCACGTCAAA | | | 96 | 45 | 96 | 597 | |
| | | < 0 | GCTTCAGACCTGACA | | | | | | | |
| | | | AATCA | | | | | | | |
| 111 | cg05314634 | − | CGACTCACAGACTG | 111 | 27 | 693475 | 693480 | 693475 | 69347 | − |
| | | 1 < x | GAACATTTCTGTGAT | | | 5 | 4 | 5 | 56 | |
| | | < 0 | CCGCTGTAATGCACT | | | | | | | |
| | | | GGGGGA | | | | | | | |
| 112 | cg25130381 | 0 < x | TGTGATCCCCACTAT | 112 | 2 | 704769 | 704770 | 704770 | 70477 | − |
| | | < 1 | CTCAAGCATCGTCCC | | | 99 | 48 | 47 | 048 | |
| | | | GGAGAGCTGCCTGCT | | | | | | | |
| | | | GATCG | | | | | | | |
| 113 | cg17199893 | − | TGACCTCTGCATGAT | 113 | 9 | 244115 | 244115 | 244115 | 24411 | − |
| | | 1 < x | CCCGGACTCTATGAA | | | 31 | 80 | 79 | 580 | |
| | | < 0 | TTATTGATGAGATAT | | | | | | | |
| | | | GAGCG | | | | | | | |
| 114 | cg03221837 | 0< x | CGTATGCAAAAGGC | 114 | 2 | 586443 | 586444 | 586443 | 58644 | − |
| | | < 1 | ACAATTATTCACCCA | | | 61 | 10 | 61 | 362 | |
| | | | CCAAGGTGACAGAG | | | | | | | |
| | | | AAGGCCT | | | | | | | |
| 115 | cg06785646 | − | ATTGTAAATTACCTG | 115 | 30 | 410919 | 410924 | 410924 | 41092 | + |
| | | 1 < x | TGACATCTCATTAAT | | | 5 | 4 | 3 | 44 | |
| | | < 0 | CCTCTTTTTCCTCAC | | | | | | | |
| | | | AAACG | | | | | | | |
| 116 | cg24855838 | 0 < x | AGCTGATTTCTTCAC | 116 | 26 | 111168 | 111168 | 111168 | 11116 | + |
| | | < 1 | TCCAGCAAAAGCAC | | | 33 | 82 | 81 | 882 | |
| | | | TTTAATTCCCTTTTA | | | | | | | |
| | | | GATACG | | | | | | | |
| 117 | cg27633444 | 0 < x | ATTATCCCTCTACCT | 117 | 3 | 665521 | 665521 | 665521 | 66552 | + |
| | | < 1 | TACCACCCACCAGTG | | | 30 | 79 | 78 | 179 | |
| | | | TTGTGGATTTAAGAG | | | | | | | |
| | | | AGTCG | | | | | | | |
| 118 | cg24980230 | − | TTAAAACCAGTTCTA | 118 | 14 | 287531 | 287531 | 287531 | 28753 | − |
| | | 1 < x | TCCACTGTAACAATG | | | 07 | 56 | 55 | 156 | |
| | | < 0 | ACCTGGAGCCAAAC | | | | | | | |
| | | | AAGGCG | | | | | | | |
| 119 | cg26698347 | − | CGGCTGCATTCCGAC | 119 | 27 | 693474 | 693479 | 693474 | 69347 | − |
| | | 1 < x | TCACAGACTGGAAC | | | 4 | 3 | 4 | 45 | |
| | | < 0 | ATTTCTGTGATCCGC | | | | | | | |
| | | | TGTAAT | | | | | | | |
| 120 | cg22929401 | − | CGTCTGTGGCGGTTT | 120 | 10 | 268346 | 268346 | 268346 | 26834 | − |
| | | 1 < x | TTCTATAGGTGCTAA | | | 13 | 62 | 13 | 614 | |
| | | < 0 | AATATTCCACCCTGG | | | | | | | |
| | | | TGACT | | | | | | | |
| 121 | cg18777747 | 0 < x | ATGTAATGTTCGGCA | 121 | 24 | 275874 | 275874 | 275874 | 27587 | − |
| | | < 1 | GCAGGAGGTAAATT | | | 28 | 77 | 76 | 477 | |
| | | | CCTCTCCAATTTCCA | | | | | | | |
| | | | GGCCCG | | | | | | | |
| 122 | cg11738855 | 0 < x | GATAAATTCCAGTCC | 122 | 7 | 436106 | 436107 | 436107 | 43610 | − |
| | | < 1 | ACAGACGCCTTATAA | | | 70 | 19 | 18 | 719 | |
| | | | ATTACATTTTTTTGTT | | | | | | | |
| | | | CGCG | | | | | | | |
| 123 | cg06044403 | 0 < x | CGAGTTAGACAGGT | 123 | 1 | 713762 | 713763 | 713762 | 71376 | + |
| | | < 1 | GATTAGCATAATTAG | | | 84 | 33 | 84 | 285 | |
| | | | CACCGAGCAGCTAT | | | | | | | |
| | | | ACATATG | | | | | | | |
| 124 | cg23190295 | − | CGGATCCGCTCCGAC | 124 | 27 | 503506 | 503511 | 503506 | 50350 | − |
| | | 1 < x | TCCAGGGTGATCTGC | | | 3 | 2 | 3 | 64 | |
| | | < 0 | TCAAAGGCTGAGTC | | | | | | | |
| | | | ACACAC | | | | | | | |
| 125 | cg12373771 | >1 | AGCACCAGTACAGG | 125 | 27 | 451203 | 451203 | 451203 | 45120 | − |
| | | | TCGGTGACGGCGAT | | | 46 | 95 | 94 | 395 | |
| | | | GAGGTACAGGTCCA | | | | | | | |
| | | | GCAGGCCG | | | | | | | |
| 126 | cg07547549 | >1 | GCTCAGCTCCATTGG | 126 | 24 | 332623 | 332623 | 332623 | 33262 | − |
| | | | AATGCTCCGGGCGCT | | | 14 | 63 | 62 | 363 | |
| | | | GTCCAAGGTGCTGG | | | | | | | |
| | | | AATGCG | | | | | | | |
| 127 | cg21030623 | >1 | AGACACCCACCTGTA | 127 | 22 | 500283 | 500283 | 500283 | 50028 | − |
| | | | TGAGTACGCTTGTGG | | | 00 | 49 | 48 | 349 | |
| | | | ATCTTGAGGTTCTCG | | | | | | | |
| | | | GAGCG | | | | | | | |
| 128 | cg12879445 | >1 | AACTGGACAGCACC | 128 | 8 | 625429 | 625429 | 625429 | 62542 | 1 |
| | | | ATGTCCACCAAAGC | | | 17 | 66 | 65 | 966 | |
| | | | GGAGCAGTGTAAGT | | | | | | | |
| | | | AGCAGCCG | | | | | | | |
| 129 | cg00295657 | <−1 | ACTGACCAATGGCA | 129 | 9 | 244235 | 244235 | 244235 | 24423 | − |
| | | | GAGGCAGGAATTGT | | | 09 | 58 | 57 | 558 | |
| | | | CAAATAGCACCCAG | | | | | | | |
| | | | GAGGAGCG | | | | | | | |
| 130 | cg27294582 | <−1 | CGGTGATTTACTTCC | 130 | 9 | 244404 | 244404 | 244404 | 24440 | + |
| | | | CTGCAAATGAGTTGT | | | 13 | 62 | 13 | 414 | |
| | | | TTCATATTTTGCACT | | | | | | | |
| | | | GTCTT | | | | | | | |
| 131 | cg25520488 | >1 | CGGACTCTACCTGTG | 131 | 9 | 120620 | 120621 | 120620 | 12062 | + |
| | | | GCTCAGGCATACCA | | | 91 | 40 | 91 | 092 | |
| | | | GGACAACCTGTACA | | | | | | | |
| | | | GGCAGCT | | | | | | | |
| 132 | cg16296826 | <−1 | CGCTCCCCCTCTAAT | 132 | 13 | 367291 | 367291 | 367291 | 36729 | + |
| | | | GTGTGATCTGGAAGC | | | 12 | 61 | 12 | 113 | |
| | | | TCTATAAAGCCTGAT | | | | | | | |
| | | | GTAAT | | | | | | | |
| 133 | cg14870509 | <−1 | CGGCTTGATATTTCC | 133 | 10 | 597961 | 597961 | 597961 | 59796 | − |
| | | | GAAGAATATAGTGG | | | 24 | 73 | 24 | 125 | |
| | | | GCTTTATTAGCACCA | | | | | | | |
| | | | GTTTCG | | | | | | | |
| 134 | cg02783173 | >1 | CGCTGAACCAGGAG | 134 | 32 | 325275 | 325276 | 325275 | 32527 | + |
| | | | ACAAACACGATTAC | | | 87 | 36 | 87 | 588 | |
| | | | CAGCTCCGAGCCTTG | | | | | | | |
| | | | AGTCAGA | | | | | | | |
| 135 | cg24905433 | <−1 | CGATCAGATGAGTTC | 135 | 14 | 168995 | 168995 | 168995 | 16899 | + |
| | | | CTCTTAGTGCAGGTC | | | 12 | 61 | 12 | 513 | |
| | | | AAAAAGGCTAGTTA | | | | | | | |
| | | | GCAGGA | | | | | | | |
| 136 | cg22100382 | <−1 | TTAAGCACATTTACA | 136 | 19 | 494783 | 494784 | 494784 | 49478 | + |
| | | | TTTGGTTCTTAAAAT | | | 70 | 19 | 18 | 419 | |
| | | | CCAAAATAGCCCATT | | | | | | | |
| | | | TCACG | | | | | | | |
| 137 | cg05575054 | 0 < x | CGTCTTCTTCAACTG | 137 | 28 | 249983 | 249983 | 249983 | 24998 | + |
| | | < 1 | GCTGGGCTACGCCA | | | 03 | 52 | 03 | 304 | |
| | | | ACTCGGCCTTCAACC | | | | | | | |
| | | | CCATCA | | | | | | | |
| 138 | cg05613158 | − | CGCACCGCACTCCAT | 138 | 9 | 244522 | 244522 | 244522 | 24452 | + |
| | | 1 < x | ATCGAGGATGGATT | | | 43 | 92 | 43 | 244 | |
| | | < 0 | GTTTTATGCTGATGC | | | | | | | |
| | | | AATGTG | | | | | | | |
| 139 | cg19117941 | 0 < x | AGATCTCGCCTTTCC | 139 | 22 | 846513 | 846518 | 846517 | 84651 | − |
| | | < 1 | AGATGCAAAAGTTC | | | 1 | 0 | 9 | 80 | |
| | | | AGCCCCTCTGATGTC | | | | | | | |
| | | | ATGACG | | | | | | | |
| 140 | cg02884952 | 0 < x | CGAATGCAGCTGCTC | 140 | 9 | 911836 | 911841 | 911836 | 91183 | + |
| | | < 1 | TTTGTAATTGTTTGT | | | 6 | 5 | 6 | 67 | |
| | | | GAAACTGAGTTAAA | | | | | | | |
| | | | GGGAGG | | | | | | | |
| 141 | cg16781658 | − | AGAAGGACCTTGTA | 141 | 8 | 317833 | 317838 | 317838 | 31783 | − |
| | | 1 < x | ATAAATAATATTCTG | | | 6 | 5 | 4 | 85 | |
| | | < 0 | CACATCAAATCACTT | | | | | | | |
| | | | TCACCG | | | | | | | |
| 142 | cg19965314 | − | CTCCTAGGAGCTCAC | 142 | 10 | 394966 | 394967 | 394967 | 39496 | + |
| | | 1 < x | AGCTCCAAACATCA | | | 72 | 21 | 20 | 721 | |
| | | < 0 | ATTACCATGATTATC | | | | | | | |
| | | | TACCCG | | | | | | | |
| 143 | cg20747487 | 0 < x | TTAACTGTGAAATTT | 143 | 10 | 477679 | 477679 | 477679 | 47767 | − |
| | | < 1 | TATTTCCGTTAAAAA | | | 21 | 70 | 69 | 970 | |
| | | | GCAAGCCTGTAATCA | | | | | | | |
| | | | AAACG | | | | | | | |
| 144 | cg06561106 | 0 < x | CGCTCCCCCGCCGAG | 144 | 11 | 529990 | 529990 | 529990 | 52999 | + |
| | | < 1 | CTGGGGTAGCTGATC | | | 50 | 99 | 50 | 051 | |
| | | | ACTGAGCTGAAACT | | | | | | | |
| | | | AAACGT | | | | | | | |
| 145 | cg20692569 | 0 < x | ACGTGTGGCCCAGC | 145 | 6 | 657498 | 657503 | 657503 | 65750 | + |
| | | < 1 | AGGTTGGGCATGCG | | | 2 | 1 | 0 | 31 | |
| | | | GGTCAGGTTGTAGCC | | | | | | | |
| | | | GATGCCG | | | | | | | |
| 146 | cg20621276 | 0 < x | CGCCTTCCTCATCGG | 146 | 36 | 137508 | 137512 | 137508 | 13750 | + |
| | | < 1 | CTGCATGTTCATCAA | | | 0 | 9 | 0 | 81 | |
| | | | GATGTCCCAGCCCAA | | | | | | | |
| | | | GAAGC | | | | | | | |
| 147 | cg05066539 | − | CGCTGGAGCTCCTAC | 147 | 6 | 866802 | 866806 | 866802 | 86680 | + |
| | | 1 < x | ATGGTGCACTGGAA | | | 0 | 9 | 0 | 21 | |
| | | < 0 | GAACCAGTTCGACC | | | | | | | |
| | | | ACTACAG | | | | | | | |
| 148 | cg08215831 | 0 < x | CGCTCTCTTGACAGC | 148 | 29 | 159422 | 159423 | 159422 | 15942 | + |
| | | < 1 | TCGATTGCGTGCTGC | | | 66 | 15 | 66 | 267 | |
| | | | CTCTGCTCCTGCATA | | | | | | | |
| | | | AATCA | | | | | | | |
| 149 | cg11084334 | 0 < x | CGCCATCATCAACGT | 149 | 20 | 838299 | 838304 | 838299 | 83829 | + |
| | | < 1 | GGTGGTCTTCATCCA | | | 3 | 2 | 3 | 94 | |
| | | | GCCCTACTGGGTGGG | | | | | | | |
| | | | CGACA | | | | | | | |

TABLE 4
Top5 Clock

| Site | | Co- | | SEQ | | | | | | |
|---|---|---|---|---|---|---|---|---|---|---|
| num- | | effi- | | ID | | probe | probe | | | |
| ber | CGid | cient | Sequence | NO: | Chr | Start | End | CGstart | CGend | Strand |
| Inter- | N/A | −24.53 | N/A | | | | | | | − |
| cept | | | | | | | | | | |
| 125 | cg1237 | 41.11 | AGCACCAGTACAGG | 125 | 27 | 451203 | 451203 | 451203 | 45120 | − |
| | 3771 | | TCGGTGACGGCGAT | | | 46 | 95 | 94 | 395 | |
| | | | GAGGTACAGGTCCA | | | | | | | |
| | | | GCAGGCCG | | | | | | | |
| 1 | cg0247 | 14.19 | CGCAATGTTCATCAA | 1 | 9 | 224702 | 224703 | 224702 | 22470 | + |
| | 1603 | | GCTGAGCCGAGAAG | | | 82 | 31 | 82 | 283 | |
| | | | ACATTAATAGCCTGA | | | | | | | |
| | | | TGAATA | | | | | | | |
| 2 | cg1713 | 33.61 | CGAGAGCAGCTCGA | 2 | 25 | 268232 | 268232 | 268232 | 26823 | + |
| | 6263 | | TGCACATCTGCCACT | | | 27 | 76 | 27 | 228 | |
| | | | GCTGCAACACCTCCT | | | | | | | |
| | | | CGTGCT | | | | | | | |
| 3 | cg2319 | 85.89 | TGGTTTCAAAACCGC | 3 | 36 | 201422 | 201422 | 201422 | 20142 | + |
| | 4298 | | CGAATGAAACTCAA | | | 19 | 68 | 67 | 268 | |
| | | | GAAGATGAGCCGAG | | | | | | | |
| | | | AGAACCG | | | | | | | |
| 126 | cg0754 | 19.44 | GCTCAGCTCCATTGG | 126 | 24 | 332623 | 332623 | 332623 | 33262 | − |
| | 7549 | | AATGCTCCGGGCGCT | | | 14 | 63 | 62 | 363 | |
| | | | GTCCAAGGTGCTGG | | | | | | | |
| | | | AATGCG | | | | | | | |

TABLE 5
Top10 Clock

| Site | | Co- | | SEQ | | | | | | |
|---|---|---|---|---|---|---|---|---|---|---|
| num | | effi- | | ID | | probe | probe | | | |
| ber | CGid | cient | Sequence | NO: | Chr | Start | End | CGstart | CGend | Strand |
| Inter- | N/A | −27.89 | N/A | | | | | | | |
| cept | | | | | | | | | | |
| 125 | cg1237 | >1 | AGCACCAGTACAGG | 125 | 27 | 451203 | 451203 | 451203 | 45120 | − |
| | 3771 | | TCGGTGACGGCGAT | | | 46 | 95 | 94 | 395 | |
| | | | GAGGTACAGGTCCA | | | | | | | |
| | | | GCAGGCCG | | | | | | | |
| 1 | cg0247 | >1 | CGCAATGTTCATCAA | 1 | 9 | 224702 | 224703 | 224702 | 22470 | + |
| | 1603 | | GCTGAGCCGAGAAG | | | 82 | 31 | 82 | 283 | |
| | | | ACATTAATAGCCTGA | | | | | | | |
| | | | TGAATA | | | | | | | |
| 2 | cg1713 | >1 | CGAGAGCAGCTCGA | 2 | 25 | 268232 | 268232 | 268232 | 26823 | + |
| | 6263 | | TGCACATCTGCCACT | | | 27 | 76 | 27 | 228 | |
| | | | GCTGCAACACCTCCT | | | | | | | |
| | | | CGTGCT | | | | | | | |
| 3 | cg2319 | >1 | TGGTTTCAAAACCGC | 3 | 36 | 201422 | 201422 | 201422 | 20142 | + |
| | 4298 | | CGAATGAAACTCAA | | | 19 | 68 | 67 | 268 | |
| | | | GAAGATGAGCCGAG | | | | | | | |
| | | | AGAACCG | | | | | | | |
| 126 | cg0754 | >1 | GCTCAGCTCCATTGG | 126 | 24 | 332623 | 332623 | 332623 | 33262 | − |
| | 7549 | | AATGCTCCGGGCGCT | | | 14 | 63 | 62 | 363 | |
| | | | GTCCAAGGTGCTGG | | | | | | | |
| | | | AATGCG | | | | | | | |
| 127 | cg2103 | >1 | AGACACCCACCTGTA | 127 | 22 | 500283 | 500283 | 500283 | 50028 | − |
| | 0623 | | TGAGTACGCTTGTGG | | | 00 | 49 | 48 | 349 | |
| | | | ATCTTGAGGTTCTCG | | | | | | | |
| | | | GAGCG | | | | | | | |
| 4 | cg1443 | >1 | AAGCAAATCAGATT | 4 | 37 | 232437 | 232437 | 232437 | 23243 | + |
| | 5444 | | CCAGGCTGCTGCCAG | | | 29 | 78 | 77 | 778 | |
| | | | GTGTTGTCTCGTCTT | | | | | | | |
| | | | CCGCCG | | | | | | | |
| 5 | cg0751 | >1 | CGACAGGCAGGTCA | 5 | 36 | 201422 | 201422 | 201422 | 20142 | + |
| | 7669 | | AGATTTGGTTTCAAA | | | 00 | 49 | 00 | 201 | |
| | | | ACCGCCGAATGAAA | | | | | | | |
| | | | CTCAAGA | | | | | | | |
| 6 | cg1752 | <−1 | CGGCACAGACTCCA | 6 | 9 | 457761 | 457761 | 457761 | 45776 | + |
| | 6723 | | GGGTTCACGCATATG | | | 33 | 82 | 33 | 134 | |
| | | | GCCAGACAGATGTG | | | | | | | |
| | | | TCTGGCT | | | | | | | |
| 7 | cg0733 | >1 | CGGAGGAGCTGACC | 7 | 21 | 423349 | 423350 | 423350 | 42335 | − |
| | 0957 | | AATTGGTCATGGTGC | | | 66 | 15 | 14 | 015 | |
| | | | TGAAACCTTTGTGGG | | | | | | | |
| | | | GAATCG | | | | | | | |

TABLE 6
Top30 Clock

| Site | | Co- | | SEQ | | | | | | |
|---|---|---|---|---|---|---|---|---|---|---|
| num- | | effi- | | ID | | probe | probe | | | |
| ber | CGid | cient | Sequence | NO: | Chr | Start | End | CGstart | CGend | Strand |
| Inter- | N/A | −29.01 | N/A | | | | | | | |
| cept | | | | | | | | | | |
| 125 | cg1237 | >1 | AGCACCAGTACAGG | 125 | 27 | 451203 | 451203 | 451203 | 45120 | − |
| | 3771 | | TCGGTGACGGCGAT | | | 46 | 95 | 94 | 395 | |
| | | | GAGGTACAGGTCCA | | | | | | | |
| | | | GCAGGCCG | | | | | | | |
| 1 | cg0247 | >1 | CGCAATGTTCATCAA | 1 | 9 | 224702 | 224703 | 224702 | 22470 | + |
| | 1603 | | GCTGAGCCGAGAAG | | | 82 | 31 | 82 | 283 | |
| | | | ACATTAATAGCCTGA | | | | | | | |
| | | | TGAATA | | | | | | | |
| 2 | cg1713 | >1 | CGAGAGCAGCTCGA | 2 | 25 | 268232 | 268232 | 268232 | 26823 | + |
| | 6263 | | TGCACATCTGCCACT | | | 27 | 76 | 27 | 228 | |
| | | | GCTGCAACACCTCCT | | | | | | | |
| | | | CGTGCT | | | | | | | |
| 3 | cg2319 | >1 | TGGTTTCAAAACCGC | 3 | 36 | 201422 | 201422 | 201422 | 20142 | + |
| | 4298 | | CGAATGAAACTCAA | | | 19 | 68 | 67 | 268 | |
| | | | GAAGATGAGCCGAG | | | | | | | |
| | | | AGAACCG | | | | | | | |
| 126 | cg0754 | >1 | GCTCAGCTCCATTGG | 126 | 24 | 332623 | 332623 | 332623 | 33262 | − |
| | 7549 | | AATGCTCCGGGCGCT | | | 14 | 63 | 62 | 363 | |
| | | | GTCCAAGGTGCTGG | | | | | | | |
| | | | AATGCG | | | | | | | |
| 127 | cg2103 | >1 | AGACACCCACCTGTA | 127 | 22 | 500283 | 500283 | 500283 | 50028 | − |
| | 0623 | | TGAGTACGCTTGTGG | | | 00 | 49 | 48 | 349 | |
| | | | ATCTTGAGGTTCTCG | | | | | | | |
| | | | GAGCG | | | | | | | |
| 4 | cg1443 | >1 | AAGCAAATCAGATT | 4 | 37 | 232437 | 232437 | 232437 | 23243 | + |
| | 5444 | | CCAGGCTGCTGCCAG | | | 29 | 78 | 77 | 778 | |
| | | | GTGTTGTCTCGTCTT | | | | | | | |
| | | | CCGCCG | | | | | | | |
| 5 | cg0751 | >1 | CGACAGGCAGGTCA | 5 | 36 | 201422 | 201422 | 201422 | 20142 | + |
| | 7669 | | AGATTTGGTTTCAAA | | | 00 | 49 | 00 | 201 | |
| | | | ACCGCCGAATGAAA | | | | | | | |
| | | | CTCAAGA | | | | | | | |
| 6 | cg1752 | <−1 | CGGCACAGACTCCA | 6 | 9 | 457761 | 457761 | 457761 | 45776 | + |
| | 6723 | | GGGTTCACGCATATG | | | 33 | 82 | 33 | 134 | |
| | | | GCCAGACAGATGTG | | | | | | | |
| | | | TCTGGCT | | | | | | | |
| 7 | cg0733 | >1 | CGGAGGAGCTGACC | 7 | 21 | 423349 | 423350 | 423350 | 42335 | − |
| | 0957 | | AATTGGTCATGGTGC | | | 66 | 15 | 14 | 015 | |
| | | | TGAAACCTTTGTGGG | | | | | | | |
| | | | GAATCG | | | | | | | |
| 128 | cg1287 | >1 | AACTGGACAGCACC | 128 | 8 | 625429 | 625429 | 625429 | 62542 | + |
| | 9445 | | ATGTCCACCAAAGC | | | 17 | 66 | 65 | 966 | |
| | | | GGAGCAGTGTAAGT | | | | | | | |
| | | | AGCAGCCG | | | | | | | |
| 129 | cg0029 | <-1 | ACTGACCAATGGCA | 129 | 9 | 244235 | 244235 | 244235 | 24423 | − |
| | 5657 | | GAGGCAGGAATTGT | | | 09 | 58 | 57 | 558 | |
| | | | CAAATAGCACCCAG | | | | | | | |
| | | | GAGGAGCG | | | | | | | |
| 8 | cg1527 | >1 | TCCCAGCCTGGGCAA | 8 | 24 | 264135 | 264135 | 264135 | 26413 | − |
| | 9950 | | AACTTTGATGATAAA | | | 20 | 69 | 68 | 569 | |
| | | | TCAAGCGGCAGATC | | | | | | | |
| | | | CCTCCG | | | | | | | |
| 130 | cg2729 | <−1 | CGGTGATTTACTTCC | 130 | 9 | 244404 | 244404 | 244404 | 24440 | + |
| | 4582 | | CTGCAAATGAGTTGT | | | 13 | 62 | 13 | 414 | |
| | | | TTCATATTTTGCACT | | | | | | | |
| | | | GTCTT | | | | | | | |
| 9 | cg0441 | <−1 | CGAAATTAGGTGGTC | 9 | 8 | 433591 | 433591 | 433591 | 43359 | + |
| | 1283 | | GTTGGAATCCTGATC | | | 26 | 75 | 26 | 127 | |
| | | | GCAGTAAAGTGGTC | | | | | | | |
| | | | CTTGGT | | | | | | | |
| 10 | cg1287 | >1 | CGTAGCCGAAGCTG | 10 | 32 | 309177 | 309177 | 309177 | 30917 | − |
| | 0762 | | GAGTGCTGCTTTGCT | | | 42 | 91 | 42 | 743 | |
| | | | TTCAGTCTCAGGCTG | | | | | | | |
| | | | GCCAGG | | | | | | | |
| 11 | cg0479 | >1 | CGCTGTCCAGCTGCA | 11 | 23 | 517036 | 517037 | 517036 | 51703 | − |
| | 4444 | | GCTGCGCCTGAACCT | | | 58 | 07 | 58 | 659 | |
| | | | GAAAGGACAAGGGC | | | | | | | |
| | | | GTCACG | | | | | | | |
| 131 | cg2552 | >1 | CGGACTCTACCTGTG | 131 | 9 | 120620 | 120621 | 120620 | 12062 | + |
| | 0488 | | GCTCAGGCATACCA | | | 91 | 40 | 91 | 092 | |
| | | | GGACAACCTGTACA | | | | | | | |
| | | | GGCAGCT | | | | | | | |
| 12 | cg0971 | >1 | CGGTGACCCACAGG | 12 | 24 | 276233 | 276233 | 276233 | 27623 | + |
| | 8073 | | TACTGCGCATGGAAC | | | 04 | 53 | 04 | 305 | |
| | | | CTAATGACCAGTCAC | | | | | | | |
| | | | TCAATT | | | | | | | |
| 13 | cg0085 | <−1 | AACCAGGTGCAAGC | 13 | 7 | 165441 | 165441 | 165441 | 16544 | − |
| | 7814 | | TGGGAAACAGTCCC | | | 08 | 57 | 56 | 157 | |
| | | | ACATTCCTTACAGCC | | | | | | | |
| | | | AGCAACG | | | | | | | |
| 14 | cg1380 | <−1 | CGTCTGGGATGGGG | 14 | 11 | 620677 | 620677 | 620677 | 62067 | + |
| | 4575 | | AAAGACCAACCAGT | | | 27 | 76 | 27 | 728 | |
| | | | TGGGGCTTTCTCCCA | | | | | | | |
| | | | GGGCTCC | | | | | | | |
| 15 | cg2017 | >1 | ACAGATGCAAATAC | 15 | 28 | 242467 | 242468 | 242468 | 24246 | + |
| | 3540 | | TCCAAAGAAGTGTC | | | 54 | 03 | 02 | 803 | |
| | | | GGGCACTGTTCGGGC | | | | | | | |
| | | | TTGACCG | | | | | | | |
| 16 | cg0460 | <−1 | AAGTCCGAGAGGGG | 16 | 35 | 201260 | 201265 | 201265 | 20126 | − |
| | 7114 | | GCCTTTCACATGACA | | | 6 | 5 | 4 | 55 | |
| | | | TCATAAAAGCCTGAT | | | | | | | |
| | | | TTATCG | | | | | | | |
| 17 | cg2389 | <−1 | CGAGTTTGGGGGCAT | 17 | 12 | 569387 | 569387 | 569387 | 56938 | + |
| | 9560 | | GACAGTACATCTGAC | | | 09 | 58 | 09 | 710 | |
| | | | CCCTTTGGGGTAAAA | | | | | | | |
| | | | TTTGA | | | | | | | |
| 18 | cg2134 | <−1 | ATGGCCAGTCATTTT | 18 | 26 | 111582 | 111582 | 111582 | 11158 | − |
| | 5677 | | GTTCACAACTTTGCA | | | 14 | 63 | 62 | 263 | |
| | | | GCAAGCAGGAGCAA | | | | | | | |
| | | | AAGCCG | | | | | | | |
| 132 | cg1629 | <−1 | CGCTCCCCCTCTAAT | 132 | 13 | 367291 | 367291 | 367291 | 36729 | + |
| | 6826 | | GTGTGATCTGGAAGC | | | 12 | 61 | 12 | 113 | |
| | | | TCTATAAAGCCTGAT | | | | | | | |
| | | | GTAAT | | | | | | | |
| 19 | cg1873 | <−1 | TAAACTCCTATGTAT | 19 | 16 | 470876 | 470877 | 470877 | 47087 | + |
| | 7166 | | GTTCACATCTATGAT | | | 71 | 20 | 19 | 720 | |
| | | | CTGCTAACCATTGCT | | | | | | | |
| | | | ACTCG | | | | | | | |
| 20 | cg2317 | >1 | CGGATATAATTATCG | 20 | 36 | 167019 | 167019 | 167019 | 16701 | + |
| | 7112 | | AGGAGTCAAGATGT | | | 20 | 69 | 20 | 921 | |
| | | | TATGCTAAAAACTAT | | | | | | | |
| | | | GCATTG | | | | | | | |
| 21 | cg0003 | >1 | GTCTTCCTTTCCTGT | 21 | 13 | 150688 | 150693 | 150693 | 15069 | − |
| | 8955 | | ACTGACAAGCTGAA | | | 4 | 3 | 2 | 33 | |
| | | | CAGACGCACCTTGTT | | | | | | | |
| | | | GGTTCG | | | | | | | |
| 22 | cg1019 | >1 | CGTGTCTGTTTGCAG | 22 | 20 | 283840 | 283840 | 283840 | 28384 | + |
| | 6526 | | CACCCCTGGGGCGA | | | 35 | 84 | 35 | 036 | |
| | | | GCTGTGGCTTCCTGT | | | | | | | |
| | | | AACATG | | | | | | | |

TABLE 7
Top50 Clock

| Site | | Co- | | SEQ | | | | | | |
|---|---|---|---|---|---|---|---|---|---|---|
| num- | | effi- | | ID | | probe | probe | | | |
| ber | CGid | cient | Sequence | NO: | Chr | Start | End | CGstart | CGend | Strand |
| Inter- | N/A | −16.742 | N/A | | | | | | | |
| cept | | | | | | | | | | |
| 125 | cg1237 | >1 | AGCACCAGTACAGG | 125 | 27 | 451203 | 451203 | 451203 | 45120 | − |
| | 3771 | | TCGGTGACGGCGAT | | | 46 | 95 | 94 | 395 | |
| | | | GAGGTACAGGTCCA | | | | | | | |
| | | | GCAGGCCG | | | | | | | |
| 1 | cg0247 | >1 | CGCAATGTTCATCAA | 1 | 9 | 224702 | 224703 | 224702 | 22470 | + |
| | 1603 | | GCTGAGCCGAGAAG | | | 82 | 31 | 82 | 283 | |
| | | | ACATTAATAGCCTGA | | | | | | | |
| | | | TGAATA | | | | | | | |
| 2 | cg1713 | >1 | CGAGAGCAGCTCGA | 2 | 25 | 268232 | 268232 | 268232 | 26823 | − |
| | 6263 | | TGCACATCTGCCACT | | | 27 | 76 | 27 | 228 | |
| | | | GCTGCAACACCTCCT | | | | | | | |
| | | | CGTGCT | | | | | | | |
| 3 | cg2319 | >1 | TGGTTTCAAAACCGC | 3 | 36 | 201422 | 201422 | 201422 | 20142 | + |
| | 4298 | | CGAATGAAACTCAA | | | 19 | 68 | 67 | 268 | |
| | | | GAAGATGAGCCGAG | | | | | | | |
| | | | AGAACCG | | | | | | | |
| 126 | cg0754 | >1 | GCTCAGCTCCATTGG | 126 | 24 | 332623 | 332623 | 332623 | 33262 | − |
| | 7549 | | AATGCTCCGGGCGCT | | | 14 | 63 | 62 | 363 | |
| | | | GTCCAAGGTGCTGG | | | | | | | |
| | | | AATGCG | | | | | | | |
| 127 | cg2103 | >1 | AGACACCCACCTGTA | 127 | 22 | 500283 | 500283 | 500283 | 50028 | − |
| | 0623 | | TGAGTACGCTTGTGG | | | 00 | 49 | 48 | 349 | |
| | | | ATCTTGAGGTTCTCG | | | | | | | |
| | | | GAGCG | | | | | | | |
| 4 | cg1443 | >1 | AAGCAAATCAGATT | 4 | 37 | 232437 | 232437 | 232437 | 23243 | + |
| | 5444 | | CCAGGCTGCTGCCAG | | | 29 | 78 | 77 | 778 | |
| | | | GTGTTGTCTCGTCTT | | | | | | | |
| | | | CCGCCG | | | | | | | |
| 5 | cg0751 | >1 | CGACAGGCAGGTCA | 5 | 36 | 201422 | 201422 | 201422 | 20142 | + |
| | 7669 | | AGATTTGGTTTCAAA | | | 00 | 49 | 00 | 201 | |
| | | | ACCGCCGAATGAAA | | | | | | | |
| | | | CTCAAGA | | | | | | | |
| 6 | cg1752 | <−1 | CGGCACAGACTCCA | 6 | 9 | 457761 | 457761 | 457761 | 45776 | + |
| | 6723 | | GGGTTCACGCATATG | | | 33 | 82 | 33 | 134 | |
| | | | GCCAGACAGATGTG | | | | | | | |
| | | | TCTGGCT | | | | | | | |
| 7 | cg0733 | >1 | CGGAGGAGCTGACC | 7 | 21 | 423349 | 423350 | 423350 | 42335 | − |
| | 0957 | | AATTGGTCATGGTGC | | | 66 | 15 | 14 | 015 | |
| | | | TGAAACCTTTGTGGG | | | | | | | |
| | | | GAATCG | | | | | | | |
| 128 | cg1287 | >1 | AACTGGACAGCACC | 128 | 8 | 625429 | 625429 | 625429 | 62542 | + |
| | 9445 | | ATGTCCACCAAAGC | | | 17 | 66 | 65 | 966 | |
| | | | GGAGCAGTGTAAGT | | | | | | | |
| | | | AGCAGCCG | | | | | | | |
| 129 | cg0029 | <−1 | ACTGACCAATGGCA | 129 | 9 | 244235 | 244235 | 244235 | 24423 | − |
| | 5657 | | GAGGCAGGAATTGT | | | 09 | 58 | 57 | 558 | |
| | | | CAAATAGCACCCAG | | | | | | | |
| | | | GAGGAGCG | | | | | | | |
| 8 | cg1527 | >1 | TCCCAGCCTGGGCAA | 8 | 24 | 264135 | 264135 | 264135 | 26413 | − |
| | 9950 | | AACTTTGATGATAAA | | | 20 | 69 | 68 | 569 | |
| | | | TCAAGCGGCAGATC | | | | | | | |
| | | | CCTCCG | | | | | | | |
| 130 | cg2729 | <−1 | CGGTGATTTACTTCC | 130 | 9 | 244404 | 244404 | 244404 | 24440 | + |
| | 4582 | | CTGCAAATGAGTTGT | | | 13 | 62 | 13 | 414 | |
| | | | TTCATATTTTGCACT | | | | | | | |
| | | | GTCTT | | | | | | | |
| 9 | cg0441 | <−1 | CGAAATTAGGTGGTC | 9 | 8 | 433591 | 433591 | 433591 | 43359 | + |
| | 1283 | | GTTGGAATCCTGATC | | | 26 | 75 | 26 | 127 | |
| | | | GCAGTAAAGTGGTC | | | | | | | |
| | | | CTTGGT | | | | | | | |
| 10 | cg1287 | >1 | CGTAGCCGAAGCTG | 10 | 32 | 309177 | 309177 | 309177 | 30917 | − |
| | 0762 | | GAGTGCTGCTTTGCT | | | 42 | 91 | 42 | 743 | |
| | | | TTCAGTCTCAGGCTG | | | | | | | |
| | | | GCCAGG | | | | | | | |
| 11 | cg0479 | >1 | CGCTGTCCAGCTGCA | 11 | 23 | 517036 | 517037 | 517036 | 51703 | − |
| | 4444 | | GCTGCGCCTGAACCT | | | 58 | 07 | 58 | 659 | |
| | | | GAAAGGACAAGGGC | | | | | | | |
| | | | GTCACG | | | | | | | |
| 131 | cg2552 | >1 | CGGACTCTACCTGTG | 131 | 9 | 120620 | 120621 | 120620 | 12062 | + |
| | 0488 | | GCTCAGGCATACCA | | | 91 | 40 | 91 | 092 | |
| | | | GGACAACCTGTACA | | | | | | | |
| | | | GGCAGCT | | | | | | | |
| 12 | cg0971 | >1 | CGGTGACCCACAGG | 12 | 24 | 276233 | 276233 | 276233 | 27623 | + |
| | 8073 | | TACTGCGCATGGAAC | | | 04 | 53 | 04 | 305 | |
| | | | CTAATGACCAGTCAC | | | | | | | |
| | | | TCAATT | | | | | | | |
| 13 | cg0085 | <−1 | AACCAGGTGCAAGC | 13 | 7 | 165441 | 165441 | 165441 | 16544 | − |
| | 7814 | | TGGGAAACAGTCCC | | | 08 | 57 | 56 | 157 | |
| | | | ACATTCCTTACAGCC | | | | | | | |
| | | | AGCAACG | | | | | | | |
| 14 | cg1380 | <−1 | CGTCTGGGATGGGG | 14 | 11 | 620677 | 620677 | 620677 | 62067 | + |
| | 4575 | | AAAGACCAACCAGT | | | 27 | 76 | 27 | 728 | |
| | | | TGGGGCTTTCTCCCA | | | | | | | |
| | | | GGGCTCC | | | | | | | |
| 15 | cg2017 | >1 | ACAGATGCAAATAC | 15 | 28 | 242467 | 242468 | 242468 | 24246 | + |
| | 3540 | | TCCAAAGAAGTGTC | | | 54 | 03 | 02 | 803 | |
| | | | GGGCACTGTTCGGGC | | | | | | | |
| | | | TTGACCG | | | | | | | |
| 16 | cg0460 | <−1 | AAGTCCGAGAGGGG | 16 | 35 | 201260 | 201265 | 201265 | 20126 | − |
| | 7114 | | GCCTTTCACATGACA | | | 6 | 5 | 4 | 55 | |
| | | | TCATAAAAGCCTGAT | | | | | | | |
| | | | TTATCG | | | | | | | |
| 17 | cg2389 | <−1 | CGAGTTTGGGGGCAT | 17 | 12 | 569387 | 569387 | 569387 | 56938 | + |
| | 9560 | | GACAGTACATCTGAC | | | 09 | 58 | 09 | 710 | |
| | | | CCCTTTGGGGTAAAA | | | | | | | |
| | | | TTTGA | | | | | | | |
| 18 | cg2134 | >1 | ATGGCCAGTCATTTT | 18 | 26 | 111582 | 111582 | 111582 | 11158 | − |
| | 5677 | | GTTCACAACTTTGCA | | | 14 | 63 | 62 | 263 | |
| | | | GCAAGCAGGAGCAA | | | | | | | |
| | | | AAGCCG | | | | | | | |
| 132 | cg1629 | <−1 | CGCTCCCCCTCTAAT | 132 | 13 | 367291 | 367291 | 367291 | 36729 | + |
| | 6826 | | GTGTGATCTGGAAGC | | | 12 | 61 | 12 | 113 | |
| | | | TCTATAAAGCCTGAT | | | | | | | |
| | | | GTAAT | | | | | | | |
| 19 | cg1873 | <−1 | TAAACTCCTATGTAT | 19 | 16 | 470876 | 470877 | 470877 | 47087 | + |
| | 7166 | | GTTCACATCTATGAT | | | 71 | 20 | 19 | 720 | |
| | | | CTGCTAACCATTGCT | | | | | | | |
| | | | ACTCG | | | | | | | |
| 20 | cg2317 | 0 < x < | CGGATATAATTATCG | 20 | 36 | 167019 | 167019 | 167019 | 16701 | + |
| | 7112 | 1 | AGGAGTCAAGATGT | | | 20 | 69 | 20 | 921 | |
| | | | TATGCTAAAAACTAT | | | | | | | |
| | | | GCATTG | | | | | | | |
| 21 | cg0003 | >1 | GTCTTCCTTTCCTGT | 21 | 13 | 150688 | 150693 | 150693 | 15069 | − |
| | 8955 | | ACTGACAAGCTGAA | | | 4 | 3 | 2 | 33 | |
| | | | CAGACGCACCTTGTT | | | | | | | |
| | | | GGTTCG | | | | | | | |
| 22 | cg1019 | >1 | CGTGTCTGTTTGCAG | 22 | 20 | 283840 | 283840 | 283840 | 28384 | + |
| | 6526 | | CACCCCTGGGGCGA | | | 35 | 84 | 35 | 036 | |
| | | | GCTGTGGCTTCCTGT | | | | | | | |
| | | | AACATG | | | | | | | |
| 23 | cg0121 | >1 | CGAAGGTGAACTTCT | 23 | 27 | 383895 | 383896 | 383895 | 38389 | − |
| | 3012 | | TGATCTGGATGACCA | | | 90 | 39 | 90 | 591 | |
| | | | GGTAGTCAGGGAAT | | | | | | | |
| | | | GAGGCA | | | | | | | |
| 133 | cg1487 | <−1 | CGGCTTGATATTTCC | 133 | 10 | 597961 | 597961 | 597961 | 59796 | − |
| | 0509 | | GAAGAATATAGTGG | | | 24 | 73 | 24 | 125 | |
| | | | GCTTTATTAGCACCA | | | | | | | |
| | | | GTTTCG | | | | | | | |
| 24 | cg1931 | − | CGGATCTGAGCACTT | 24 | 36 | 166919 | 166919 | 166919 | 16691 | + |
| | 7509 | 1 < x < | GAGACTACCATTTAA | | | 23 | 72 | 23 | 924 | |
| | | 0 | TCAAATGAATCAGTA | | | | | | | |
| | | | ACTAA | | | | | | | |
| 134 | cg0278 | − | CGCTGAACCAGGAG | 134 | 32 | 325275 | 325276 | 325275 | 32527 | + |
| | 3173 | 1 < x < | ACAAACACGATTAC | | | 87 | 36 | 87 | 588 | |
| | | 0 | CAGCTCCGAGCCTTG | | | | | | | |
| | | | AGTCAGA | | | | | | | |
| 25 | cg2763 | <−1 | CGCCATCCCAGGTGG | 25 | 30 | 252982 | 252986 | 252982 | 25298 | + |
| | 7204 | | TTGAGTTCAGCCAGG | | | 0 | 9 | 0 | 21 | |
| | | | TTGAGCACAACACA | | | | | | | |
| | | | GATGCC | | | | | | | |
| 26 | cg2209 | <−1 | GTGGAAGGCGGCGT | 26 | 31 | 294273 | 294273 | 294273 | 29427 | + |
| | 4235 | | GAAGCGGCGGCTCG | | | 11 | 60 | 59 | 360 | |
| | | | TGCTGGCATCTACGG | | | | | | | |
| | | | GGATACG | | | | | | | |
| 27 | cg1828 | >1 | CGCGTACACCCAGA | 27 | 3 | 196859 | 196860 | 196859 | 19685 | − |
| | 7975 | | CATCTTCGGGCTGCT | | | 57 | 06 | 57 | 958 | |
| | | | ATTGGATTGACTTTG | | | | | | | |
| | | | AAGGTT | | | | | | | |
| 28 | cg1548 | <−1 | GGGCCAGTGTCGGG | 28 | 18 | 517878 | 517878 | 517878 | 51787 | + |
| | 6794 | | ACAGTTGTCAGCAG | | | 06 | 55 | 54 | 855 | |
| | | | GGCTCCAACATGAC | | | | | | | |
| | | | GCTCATCG | | | | | | | |
| 135 | cg2490 | <−1 | CGATCAGATGAGTTC | 135 | 14 | 168995 | 168995 | 168995 | 16899 | + |
| | 5433 | | CTCTTAGTGCAGGTC | | | 12 | 61 | 12 | 513 | |
| | | | AAAAAGGCTAGTTA | | | | | | | |
| | | | GCAGGA | | | | | | | |
| 29 | cg2150 | >1 | CCATGTACTTGAGAT | 29 | 3 | 198836 | 198836 | 198836 | 19883 | − |
| | 8838 | | TTCTCATGACATCAT | | | 49 | 98 | 97 | 698 | |
| | | | CATTACCTTGGTCTC | | | | | | | |
| | | | CCGCG | | | | | | | |
| 136 | cg2210 | <−1 | TTAAGCACATTTACA | 136 | 19 | 494783 | 494784 | 494784 | 49478 | + |
| | 0382 | | TTTGGTTCTTAAAAT | | | 70 | 19 | 18 | 419 | |
| | | | CCAAAATAGCCCATT | | | | | | | |
| | | | TCACG | | | | | | | |
| 30 | cg0031 | >1 | TCAGCCAGCAGAGA | 30 | 20 | 405021 | 405022 | 405022 | 40502 | − |
| | 2241 | | GATTGCAGCCTTCTT | | | 93 | 42 | 41 | 242 | |
| | | | CCCTGACTTCAGTGA | | | | | | | |
| | | | ACAGCG | | | | | | | |
| 31 | cg1735 | <−1 | AAAATAAAACTCAG | 31 | 12 | 117734 | 117734 | 117734 | 11773 | − |
| | 6865 | | GGACCAACATCTGCT | | | 24 | 73 | 72 | 473 | |
| | | | TCCTTTGCACTTGCT | | | | | | | |
| | | | CCGTCG | | | | | | | |
| 32 | cg2241 | <−1 | CGCACTCCCTGTGAC | 32 | 38 | 124319 | 124319 | 124319 | 12431 | − |
| | 9039 | | CCAGGACGTCATGAT | | | 01 | 50 | 01 | 902 | |
| | | | GCCTGTCTGTTTTCT | | | | | | | |
| | | | CAATG | | | | | | | |
| 33 | cg2385 | >1 | CGAAGTAAAAAATG | 33 | 8 | 411284 | 411285 | 411284 | 41128 | + |
| | 2530 | | TTGATCTTGCATCAC | | | 91 | 40 | 91 | 492 | |
| | | | CAGAGGAACATCAG | | | | | | | |
| | | | AAGCACC | | | | | | | |
| 34 | cg0196 | >1 | CGCAGGGAGAGATT | 34 | 3 | 444676 | 444677 | 444676 | 44467 | − |
| | 1426 | | AAGATCTCGTTGAAA | | | 76 | 25 | 76 | 677 | |
| | | | AGGAATAAAAATAA | | | | | | | |
| | | | CATCATC | | | | | | | |
| 35 | cg1882 | >1 | CGACGTGTCCATCCT | 35 | 8 | 388111 | 388111 | 388111 | 38811 | + |
| | 4846 | | GACCATCGAGGATG | | | 43 | 92 | 43 | 144 | |
| | | | GCATCTTCGAGGTGA | | | | | | | |
| | | | AGTCCA | | | | | | | |
| 36 | cg0269 | >1 | CGAGTGGCACATCC | 36 | 12 | 126949 | 126949 | 126949 | 12694 | + |
| | 1325 | | GCTCCAACCAGCAG | | | 27 | 76 | 27 | 928 | |
| | | | CTGGTGCCCAGCTAC | | | | | | | |
| | | | TCTGAGG | | | | | | | |
| 37 | cg0636 | >] | GGTCATCCACCTGCT | 7 | 25 | 268238 | 268238 | 268238 | 26823 | − |
| | 3517 | | GCAGATGGGGCAGG | | | 09 | 58 | 57 | 858 | |
| | | | TGTGGAGGTAAGAG | | | | | | | |
| | | | CACTGCG | | | | | | | |
| 38 | cg0847 | − | TTAGCACAGTTTAAC | 38 | 2 | 324453 | 324453 | 324453 | 32445 | + |
| | 6750 | 1 < x < | TCCACCCTCATTTAA | | | 23 | 72 | 71 | 372 | |
| | | 0 | ACTTCCTTTGATTCT | | | | | | | |
| | | | TTCCG | | | | | | | |

1. A method for determining a mortality risk and/or probability of a healthy lifespan of a dog; said method comprising:
a) providing a DNA methylation profile from a sample obtained from the dog; and
b) determining a mortality risk and/or probability of a healthy lifespan for the dog using the DNA methylation profile; wherein the DNA methylation profile comprises at least one methylation site as listed in Table 3.

2. The method according to claim 1, wherein the determining the mortality risk and/or probability of a healthy lifespan for the dog further comprises combining the DNA methylation profile with one or more of the chronological age, breed and/or sex of the dog.

3. The method according to claim 1, wherein a lifestyle regime, dietary regime or therapeutic intervention is selected based on a determination that the dog has an increased mortality risk and/or reduced probability of a healthy lifespan compared to its chronological age.

4. The method according to claim 3, wherein the lifestyle regime, dietary regime or therapeutic intervention is a dietary intervention.

5. The method according to claim 4, wherein the dietary intervention is a calorie-restricted diet, a senior diet or a low protein diet.

6. The method according to claim 1, wherein the sample is a blood sample.

7. The method according to claim 1, wherein DNA methylation is determined using a method which comprises one or more of the following steps:
(i) (a) treating the sample DNA with APOBEC or bisulfite conversion to deaminate cytosines; (b) a capture-based enrichment; and/or (c) high throughput sequencing;
(ii) (a) treating the sample DNA by bisulfite conversion to deaminate cytosines; and (b) microarray hybridization detection; or
(iii) de novo methylation sequencing.

8. A method for determining a biological age of a dog; said method comprising:
a) providing a DNA methylation profile from a sample obtained from the dog; and
b) determining a biological age for the dog using the DNA methylation profile, wherein the DNA methylation profile is linked to the mortality risk and/or probability of a healthy lifespan for the dog and wherein the DNA methylation profile comprises at least one methylation site as listed in Table 3.

9. The method according to claim 8 wherein a lifestyle regime, dietary regime or therapeutic intervention is selected based on a determination that the dog has an increased mortality risk and/or reduced probability of a healthy lifespan compared to its chronological age.

10. The method according to claim 9, wherein the lifestyle regime, dietary regime or therapeutic intervention is a dietary intervention.

11. The method according to claim 10, wherein the dietary intervention is a calorie-restricted diet, a senior diet or a low protein diet.

12. The method according to claim 8, wherein the sample is a blood sample.

13. The method according to claim 8, wherein DNA methylation is determined using a method which comprises one or more of the following steps:
(i) (a) treating the sample DNA with APOBEC or bisulfite conversion to deaminate cytosines; (b) a capture-based enrichment; and/or (c) high throughput sequencing;
(ii) (a) treating the sample DNA by bisulfite conversion to deaminate cytosines; and (b) microarray hybridization detection; or
(iii) de novo methylation sequencing.

14. A method for selecting a lifestyle regime, dietary regime or therapeutic intervention for a dog, the method comprising:
a) providing a DNA methylation profile from a sample obtained from the dog;
b) determining a mortality risk and/or probability of a healthy lifespan for the dog using the DNA methylation profile, wherein the DNA methylation profile comprises at least one methylation site as listed in Table 3; and
c) selecting a suitable lifestyle regime, dietary regime or therapeutic intervention for the dog based on the mortality risk and/or probability of a healthy lifespan determined in step b).

15. The method according to claim 14, wherein the determining the mortality risk and/or probability of a healthy lifespan for the dog further comprises combining the DNA methylation profile with one or more of the chronological age, breed and/or sex of the dog.

16. The method according to claim 14, wherein a lifestyle regime, dietary regime or therapeutic intervention is selected based on a determination that the dog has an increased mortality risk and/or reduced probability of a healthy lifespan compared to its chronological age.

17. The method according to claim 16, wherein the lifestyle regime, dietary regime or therapeutic intervention is a dietary intervention.

18. The method according to claim 17, wherein the dietary intervention is a calorie-restricted diet, a senior diet or a low protein diet.

19. The method according to claim 14, wherein the sample is a blood sample.

20. The method according to claim 14, wherein DNA methylation is determined using a method which comprises one or more of the following steps:
(i) (a) treating the sample DNA with APOBEC or bisulfite conversion to deaminate cytosines; (b) a capture-based enrichment; and/or (c) high throughput sequencing;
(ii) (a) treating the sample DNA by bisulfite conversion to deaminate cytosines; and (b) microarray hybridization detection; or
(iii) de novo methylation sequencing.
